SEQUENCE LISTING (91 sequences)

SEQ ID NO: 1 - DNA, 2446 bp, Homo sapiens
  source 1..2446: mol_type = genomic DNA
gattaccgagtgccggggagcccggaggagccgccgacgcagccgccaccgccgccgccgccgccaccagagccgccctgtccgcgccgcgcctcggcagccggaacagggccgccgtcggggagccccaacacacggtccacagctcatcatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaa

SEQ ID NO: 2 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 3 - DNA, 23 bp, synthetic construct
  source 1..23: mol_type = other DNA
ttgtgtcattcccttttatcagg

SEQ ID NO: 4 - DNA, 23 bp, synthetic construct
  source 1..23: mol_type = other DNA
aatgtgggcaacctgataaaagg

SEQ ID NO: 5 - DNA, 23 bp, synthetic construct
  source 1..23: mol_type = other DNA
agcatctgatttgggaatgtggg

SEQ ID NO: 6 - DNA, 23 bp, synthetic construct
  source 1..23: mol_type = other DNA
aagcatctgatttgggaatgtgg

SEQ ID NO: 7 - DNA, 23 bp, synthetic construct
  source 1..23: mol_type = other DNA
aagtacaaagcatctgatttggg

SEQ ID NO: 8 - DNA, 23 bp, synthetic construct
  source 1..23: mol_type = other DNA
aaagtacaaagcatctgatttgg

SEQ ID NO: 9 - DNA, 23 bp, synthetic construct
  source 1..23: mol_type = other DNA
tgatgactgcatgcagcttttgg

SEQ ID NO: 10 - DNA, 23 bp, synthetic construct
  source 1..23: mol_type = other DNA
cctcattgtcatctacaaacggg

SEQ ID NO: 11 - DNA, 23 bp, synthetic construct
  source 1..23: mol_type = other DNA
acctcattgtcatctacaaacgg

SEQ ID NO: 12 - DNA, 23 bp, synthetic construct
  source 1..23: mol_type = other DNA
cccgtttgtagatgacaatgagg

SEQ ID NO: 13 - DNA, 23 bp, synthetic construct
  source 1..23: mol_type = other DNA
ttgtagatgacaatgaggtgagg

SEQ ID NO: 14 - DNA, 23 bp, synthetic construct
  source 1..23: mol_type = other DNA
atcaatgccttatcaattttagg

SEQ ID NO: 15 - DNA, 23 bp, synthetic construct
  source 1..23: mol_type = other DNA
ttatcaattttaggtttcttcgg

SEQ ID NO: 16 - DNA, 23 bp, synthetic construct
  source 1..23: mol_type = other DNA
tcacttgttcctgatattcccgg

SEQ ID NO: 17 - DNA, 23 bp, synthetic construct
  source 1..23: mol_type = other DNA
tcgatgtgaccgggaatatcagg

SEQ ID NO: 18 - DNA, 23 bp, synthetic construct
  source 1..23: mol_type = other DNA
gactgggctctcgatgtgaccgg

SEQ ID NO: 19 - DNA, 23 bp, synthetic construct
  source 1..23: mol_type = other DNA
gattagtagcaatgaagactggg

SEQ ID NO: 20 - DNA, 23 bp, synthetic construct
  source 1..23: mol_type = other DNA
tgattagtagcaatgaagactgg

SEQ ID NO: 21 - DNA, 23 bp, synthetic construct
  source 1..23: mol_type = other DNA
agtcttcattgctactaatcagg

SEQ ID NO: 22 - DNA, 23 bp, synthetic construct
  source 1..23: mol_type = other DNA
acctgaaacttctgttgctcagg

SEQ ID NO: 23 - DNA, 23 bp, synthetic construct
  source 1..23: mol_type = other DNA
acctgagcaacagaagtttcagg

SEQ ID NO: 24 - DNA, 23 bp, synthetic construct
  source 1..23: mol_type = other DNA
gtagcccctgttgatttagacgg

SEQ ID NO: 25 - DNA, 23 bp, synthetic construct
  source 1..23: mol_type = other DNA
cataccgtctaaatcaacagggg

SEQ ID NO: 26 - DNA, 23 bp, synthetic construct
  source 1..23: mol_type = other DNA
gcataccgtctaaatcaacaggg

SEQ ID NO: 27 - DNA, 23 bp, synthetic construct
  source 1..23: mol_type = other DNA
tgcataccgtctaaatcaacagg

SEQ ID NO: 28 - DNA, 23 bp, synthetic construct
  source 1..23: mol_type = other DNA
aacaggacattgagcaagtttgg

SEQ ID NO: 29 - DNA, 23 bp, synthetic construct
  source 1..23: mol_type = other DNA
acaggacattgagcaagtttggg

SEQ ID NO: 30 - DNA, 23 bp, synthetic construct
  source 1..23: mol_type = other DNA
ggacattgagcaagtttgggagg

SEQ ID NO: 31 - DNA, 23 bp, synthetic construct
  source 1..23: mol_type = other DNA
agttacctgtaactcaggaatgg

SEQ ID NO: 32 - DNA, 23 bp, synthetic construct
  source 1..23: mol_type = other DNA
attatccattcctgagttacagg

SEQ ID NO: 33 - DNA, 23 bp, synthetic construct
  source 1..23: mol_type = other DNA
attttagttacctgtaactcagg

SEQ ID NO: 34 - DNA, 23 bp, synthetic construct
  source 1..23: mol_type = other DNA
tgatttagacggtatgcaacagg

SEQ ID NO: 35 - DNA, 23 bp, synthetic construct
  source 1..23: mol_type = other DNA
tgagttcactgtcaactggttgg

SEQ ID NO: 36 - DNA, 23 bp, synthetic construct
  source 1..23: mol_type = other DNA
agacaaacattcaagccgcttgg

SEQ ID NO: 37 - DNA, 26 bp, synthetic construct
  source 1..26: mol_type = other DNA
caagtgactgaaacgtagccgaagaa

SEQ ID NO: 38 - DNA, 26 bp, synthetic construct
  source 1..26: mol_type = other DNA
gggaggagctattatccattcctgag

SEQ ID NO: 39 - DNA, 26 bp, synthetic construct
  source 1..26: mol_type = other DNA
ttcctgatattcccggtcacatcgag

SEQ ID NO: 40 - DNA, 26 bp, synthetic construct
  source 1..26: mol_type = other DNA
gaacaagtgactgaaacgtagccgaa

SEQ ID NO: 41 - DNA, 26 bp, synthetic construct
  source 1..26: mol_type = other DNA
aaagtacaaagcatctgatttgggaa

SEQ ID NO: 42 - DNA, 26 bp, synthetic construct
  source 1..26: mol_type = other DNA
ttgcataccgtctaaatcaacagggg

SEQ ID NO: 43 - DNA, 26 bp, synthetic construct
  source 1..26: mol_type = other DNA
gactgagcctgattagtagcaatgaa

SEQ ID NO: 44 - DNA, 26 bp, synthetic construct
  source 1..26: mol_type = other DNA
aagactgggctctcgatgtgaccggg

SEQ ID NO: 45 - DNA, 26 bp, synthetic construct
  source 1..26: mol_type = other DNA
gggaatatcaggaacaagtgactgaa

SEQ ID NO: 46 - DNA, 26 bp, synthetic construct
  source 1..26: mol_type = other DNA
gactgggctctcgatgtgaccgggaa

SEQ ID NO: 47 - DNA, 26 bp, synthetic construct
  source 1..26: mol_type = other DNA
gttgcataccgtctaaatcaacaggg

SEQ ID NO: 48 - DNA, 26 bp, synthetic construct
  source 1..26: mol_type = other DNA
agactgggctctcgatgtgaccggga

SEQ ID NO: 49 - DNA, 26 bp, synthetic construct
  source 1..26: mol_type = other DNA
acctcattgtcatctacaaacgggaa

SEQ ID NO: 50 - DNA, 26 bp, synthetic construct
  source 1..26: mol_type = other DNA
tagacggtatgcaacaggacattgag

SEQ ID NO: 51 - DNA, 26 bp, synthetic construct
  source 1..26: mol_type = other DNA
gtctaaatcaacaggggctacctgag

SEQ ID NO: 52 - DNA, 26 bp, synthetic construct
  source 1..26: mol_type = other DNA
cctgatattcccggtcacatcgagag

SEQ ID NO: 53 - DNA, 26 bp, synthetic construct
  source 1..26: mol_type = other DNA
caggacattgagcaagtttgggagga

SEQ ID NO: 54 - DNA, 26 bp, synthetic construct
  source 1..26: mol_type = other DNA
aacaggacattgagcaagtttgggag

SEQ ID NO: 55 - DNA, 26 bp, synthetic construct
  source 1..26: mol_type = other DNA
agcaacagaagtttcaggtgactgag

SEQ ID NO: 56 - DNA, 26 bp, synthetic construct
  source 1..26: mol_type = other DNA
caacaggggctacctgagcaacagaa

SEQ ID NO: 57 - DNA, 26 bp, synthetic construct
  source 1..26: mol_type = other DNA
gttgatttagacggtatgcaacagga

SEQ ID NO: 58 - DNA, 26 bp, synthetic construct
  source 1..26: mol_type = other DNA
ctactaatcaggctcagtcacctgaa

SEQ ID NO: 59 - DNA, 26 bp, synthetic construct
  source 1..26: mol_type = other DNA
aggacattgagcaagtttgggaggag

SEQ ID NO: 60 - DNA, 26 bp, synthetic construct
  source 1..26: mol_type = other DNA
cctgattagtagcaatgaagactggg

SEQ ID NO: 61 - DNA, 26 bp, synthetic construct
  source 1..26: mol_type = other DNA
cattcccgtttgtagatgacaatgag

SEQ ID NO: 62 - DNA, 26 bp, synthetic construct
  source 1..26: mol_type = other DNA
caaagtacaaagcatctgatttggga

SEQ ID NO: 63 - DNA, 26 bp, synthetic construct
  source 1..26: mol_type = other DNA
ctcgatgtgaccgggaatatcaggaa

SEQ ID NO: 64 - DNA, 26 bp, synthetic construct
  source 1..26: mol_type = other DNA
tcaaagtacaaagcatctgatttggg

SEQ ID NO: 65 - DNA, 26 bp, synthetic construct
  source 1..26: mol_type = other DNA
aacctcattgtcatctacaaacggga

SEQ ID NO: 66 - DNA, 26 bp, synthetic construct
  source 1..26: mol_type = other DNA
aaacctcattgtcatctacaaacggg

SEQ ID NO: 67 - DNA, 27 bp, synthetic construct
  source 1..27: mol_type = other DNA
ggaggagctattatccattcctgtttg

SEQ ID NO: 68 - DNA, 27 bp, synthetic construct
  source 1..27: mol_type = other DNA
gacggtatgcaacaggacattgattta

SEQ ID NO: 69 - DNA, 27 bp, synthetic construct
  source 1..27: mol_type = other DNA
gcgcagacattcccgtttgtagatttg

SEQ ID NO: 70 - DNA, 27 bp, synthetic construct
  source 1..27: mol_type = other DNA
aggtgactgagcctgattagtagtttc

SEQ ID NO: 71 - DNA, 27 bp, synthetic construct
  source 1..27: mol_type = other DNA
tagatgacaatgaggtttcttcgtttg

SEQ ID NO: 72 - DNA, 27 bp, synthetic construct
  source 1..27: mol_type = other DNA
ttcggctacgtttcagtcacttgtttc

SEQ ID NO: 73 - DNA, 27 bp, synthetic construct
  source 1..27: mol_type = other DNA
agtcacttgttcctgatattccctttc

SEQ ID NO: 74 - DNA, 27 bp, synthetic construct
  source 1..27: mol_type = other DNA
tactttgatgactgcatgcagcttttg

SEQ ID NO: 75 - DNA, 20 bp, synthetic construct
  source 1..20: mol_type = other DNA
tatttgacttcagtcagcga

SEQ ID NO: 76 - DNA, 23 bp, synthetic construct
  source 1..23: mol_type = other DNA
tctcctctgacttcaacagcgac

SEQ ID NO: 77 - DNA, 22 bp, synthetic construct
  source 1..22: mol_type = other DNA
ccctgttgctgtagccaaattc

SEQ ID NO: 78 - DNA, 24 bp, synthetic construct
  source 1..24: mol_type = other DNA
tccaagtccagaagccaaactgac

SEQ ID NO: 79 - DNA, 24 bp, synthetic construct
  source 1..24: mol_type = other DNA
ggagaggatgctgctgaaggaatc

SEQ ID NO: 80 - DNA, 20 bp, synthetic construct
  source 1..20: mol_type = other DNA
ggtttggagtccctgccatt

SEQ ID NO: 81 - DNA, 21 bp, synthetic construct
  source 1..21: mol_type = other DNA
ttgcagagagtacatggagcc

SEQ ID NO: 82 - DNA, 20 bp, synthetic construct
  source 1..20: mol_type = other DNA
ctttcagaagggccaggtga

SEQ ID NO: 83 - DNA, 20 bp, synthetic construct
  source 1..20: mol_type = other DNA
gtagacaggggcgaagactg

SEQ ID NO: 84 - DNA, 22 bp, synthetic construct
  source 1..22: mol_type = other DNA
ggacaagaatacaccatctcca

SEQ ID NO: 85 - DNA, 21 bp, synthetic construct
  source 1..21: mol_type = other DNA
atactgcaggcttggaatgtc

SEQ ID NO: 86 - DNA, 20 bp, synthetic construct
  source 1..20: mol_type = other DNA
tggaggcaagatatagatct

SEQ ID NO: 87 - DNA, 23 bp, synthetic construct
  source 1..23: mol_type = other DNA
cctcattgtcatctacaaacggg

SEQ ID NO: 88 - DNA, 21 bp, synthetic construct
  source 1..21: mol_type = other DNA
gtggtctagttcaaattgtgc

SEQ ID NO: 89 - DNA, 20 bp, synthetic construct
  source 1..20: mol_type = other DNA
ggttatgctgtccatgtttc

SEQ ID NO: 90 - DNA, 28 bp, synthetic construct
  source 1..28: mol_type = other DNA
gtagtggtgccttagagcttactcatcc

SEQ ID NO: 91 - DNA, 27 bp, synthetic construct
  source 1..27: mol_type = other DNA
ctagcatgggcagtactcatgactaag

(19) United States
(12) Patent Application Publication
(10) Pub. No.: US 2025/0319206 A1
(43) Pub. Date: Oct. 16, 2025

(54) CRISPR/CAS GENE EDITING OF NEH4 AND/OR NEH5 DOMAINS IN NRF2

(71) Applicant: Christina Care Gene Editing Institute, Inc., Wilmington, DE (US)

(72) Inventors: Eric B. Kmiec, Middletown, DE (US); Kelly H. Banas, Bear, DE (US); Pawel Bialk, Wilmington, DE (US); Natalia Rivera-Torres, Bear, DE (US)

(73) Assignee: Christina Care Gene Editing Institute, Inc., Wilmington, DE (US)

(21) Appl. No.: 19/170,388

(22) Filed: Apr. 4, 2025

Related U.S. Application Data
(60) Provisional application No. 63/574,475, filed on Apr. 4, 2024.

(51) Int. Cl. A61K 48/00; C12N 9/22; C12N 15/11

(52) U.S. Cl. CPC A61K 48/0058; C12N 9/226; C12N 15/11; C12N 2310/20; C12N 2320/33

(57) ABSTRACT

Disclosed herein are Clustered Regularly Interspaced Short Palindromic Repeat (CRISPR) systems for use as a medicament, the CRISPR system comprising a guide RNA (gRNA) comprising the sequence set forth in any one of SEQ ID NO: 3-74, and a CRISPR-associated endonuclease, the gRNAs targeting the Neh4 and/or Neh5 domain of NRF2. Also disclosed herein are methods of using the aforementioned gRNAs, DNA sequences encoding such gRNAs, and vectors and pharmaceutical compositions comprising such gRNAs.

### CROSS-REFERENCE TO RELATED APPLICATIONS

This application claims the benefit of U.S. Provisional Application No. 63/574,475, filed Apr. 4, 2024, which is incorporated herein, in its entirety, by reference.

### SUBMISSION OF SEQUENCE LISTING

The Sequence Listing associated with this application is filed in electronic format via Patent Center and hereby incorporated by reference into the specification in its entirety. The name of the text file containing the Sequence Listing is 13094901820sequencelisting.xml. The size of the xml file is 83 KB, and the xml file was created on Apr. 1, 2025.

### FIELD

The present disclosure relates to compositions and methods for knocking out NRF2 to treat cancer using Clustered Regularly Interspaced Short Palindromic Repeat (CRISPR)/endonuclease gene editing.

### BACKGROUND

A core challenge of drug resistance is at the level of the gene and the number of genes that activate when drug treatment begins. Among several genes that can protect a cell against treatment is Nuclear Factor Erythroid 2-Related 2 (NRF2),¹⁻³ which has been found to be overexpressed in many solid tumors.⁴ Acting as a master regulator, NRF2 protects the tumor cell against external stress by disassociating from its pairing partner Kelch-like ECH-associated protein 1 (KEAP1) and activates a cascade of pro-carcinogenic reactions such as angiogenesis, inflammation, invasion, and metastasis but most importantly, empowers drug resistance.⁵⁻⁸ NRF2 is responsible for resistance to chemotherapy in HNC, esophageal,⁹ lung,^{10,11} glioblastoma,¹²⁻¹⁴ and pancreatic.¹⁵⁻¹⁷

Head and Neck cancer (HNC) is the 7th most commonly diagnosed cancer.¹⁸ Approximately 90% of HNC are squamous cell carcinoma, which arise from the epithelial lining of the oral cavity, pharynx, and larynx.^{19,20} The overall incidence of HNC continues to rise, with a predicted 30% increase annually by 2030.^{20,21} The conventional treatment regimen consists of a combination of chemotherapy, immunotherapy, radiation therapy and surgery. However, all these therapies have their own risks and complications.^{18,22} In fact, many patients are not able to tolerate their full chemotherapy course due to multiple and often devastating side effects. Targeted treatments and immunotherapy are the newest tool, but they are limited to patients with specific haplotypes and/or biomarkers. Development of resistance to many forms of therapy, including chemotherapy, is a major obstacle to effective cancer treatment.²³

Esophageal cancer is the eighth most diagnosed cancer and sixth leading cause of cancer death worldwide.^{24,25} While incidence of esophageal squamous carcinoma is declining, the incidence of esophageal adenocarcinoma is rising. Despite advancements in targeted therapies and immune therapy, the prognosis remains poor with an average 5-year survival rate below 20%.

Glioblastoma multiforme is the most common form of primary brain cancer in adults with a five-year survival less that 7% in the United States.²⁶ The standard treatment for patients with GBM includes surgery followed by radiation or chemotherapy, however, many patients face drug resistance. Even with innovative new therapies like gene therapy or immunotherapy, the median survival of these patients only improve by three months.²⁷

Pancreatic cancer remains the most aggressive and leading cause of cancer death. The initiation of pancreatic cancer is often subtle with very few symptoms which makes it very difficult to diagnose early. It is usually detected at advanced stages where resection is nearly impossible due to the surrounding organs and arteries and many treatment options fail due to resistance. Targeted therapy has shown some efficacy in patients with unresectable disease however the survival prognosis is minimal.

Although chemotherapy is a standard cancer treatment, chemoresistance remains a main cause of cancer mortality. Multiple studies have reported relapse and tumor resistance (>50% in HNC, 30-55% in NSCLC, 50-70% in ovarian adenocarcinomas, 20% in pediatric leukemia) after initial regression with primary standard chemo and immunotherapies. Chemotherapy often involves the combination of a platinum-based agent (e.g., cisplatin or carboplatin) and other drugs (e.g paclitaxel) with a different mechanism of action. Cisplatin or carboplatin covalently binds DNA, activates the DNA-damage response, and induces cell cycle arrest and apoptosis. The second chemotherapeutic agent can be a DNA damaging agent preventing replication such as a taxane (e.g., paclitaxel). However, alterations in regulations of key cell signaling pathways like Notch, PI3K/AKT, MAPK, JAK/STAT results in mutations in genes like TP53(53%), KRAS (27%), EGFR (17%), KEAP1(17%), CDKN2A (22%), PIK3CA (14%) that confers resistance to therapeutic agents.

Currently there are no effective means of tackling drug resistance in solid tumors, particularly chemotherapy, other than to prescribe another treatment regimen and often a more toxic drug. In many cases, the patient continues to suffer as the quality of life diminishes. Hence there is a pressing need to develop tools to manage drug resistance in general (chemoresistance) and thus enable a more widespread use of chemotherapy for better disease prognosis.

### SUMMARY

One aspect is for a Clustered Regularly Interspaced Short Palindromic Repeat (CRISPR) system for use as a medicament, the CRISPR system comprising (a) a guide RNA (gRNA) comprising the sequence set forth in any one of SEQ ID NO: 3-74, and (b) a CRISPR-associated endonuclease. In some embodiments, the gRNA comprises a trans-activated small RNA (tracrRNA) and a CRISPR RNA (crRNA). In some embodiments, the gRNA is a single gRNA. In some embodiments, the aforementioned CRISPR system is for use in treating cancer; and in some embodiments, the cancer is resistant to one or more chemotherapeutic agents. In some embodiments, the cancer is selected from the group consisting of lung cancer, head and neck cancer, esophageal cancer, glioma, pancreatic cancer, cervical cancer, breast cancer, uterine cancer, renal cell cancer, liver cancer, bladder cancer, colorectal cancer, and melanoma; and in some embodiments, the lung cancer, head and neck cancer, esophageal cancer, glioma, or pancreatic cancer is a squamous cell carcinoma. In some embodiments, the CRISPR system further comprises one or more chemotherapeutic agents; and in some embodiments, the one or more chemotherapeutic agents are selected from the group consisting of a topoisomerase II inhibitor, a mitotic inhibitor, an alkylating agent, an antimetabolite, a topoisomerase I inhibitor, a platinum compound/complex, an immunotherapy agent, and a combination thereof. In some embodiments, the CRISPR-associated endonuclease is a class 2 CRISPR-associated endonuclease; and in some embodiments, the class 2 CRISPR-associated endonuclease is Cas9 or Cas12a.

Another aspect is for a ribonucleoprotein (RNP) complex for use as a medicament, the RNP complex comprising (a) a gRNA comprising the sequence set forth in any one of SEQ ID NO: 3-74, and (b) a CRISPR-associated endonuclease. In some embodiments, the gRNA comprises a tracrRNA and a crRNA. In some embodiments, the gRNA is a single gRNA. In some embodiments, the RNP complex is for use in treating cancer; in some embodiments, the cancer is resistant to one or more chemotherapeutic agents; in some embodiments, the cancer is selected from the group consisting of lung cancer, head and neck cancer, esophageal cancer, glioma, pancreatic cancer, cervical cancer, breast cancer, uterine cancer, renal cell cancer, liver cancer, bladder cancer, colorectal cancer, and melanoma; and in some embodiments, the lung cancer, head and neck cancer, or esophageal cancer is a squamous cell carcinoma or an adenocarcinoma; the glioma is a glioblastoma; or the pancreatic cancer is a ductal adenocarcinoma. In some embodiments, the RNP complex further comprises one or more chemotherapeutic agents; and in some embodiments, the one or more chemotherapeutic agents are selected from the group consisting of a topoisomerase II inhibitor, a mitotic inhibitor, an alkylating agent, an antimetabolite, a topoisomerase I inhibitor, a platinum compound/complex, an immunotherapy agent, and a combination thereof. In some embodiments, the CRISPR-associated endonuclease is a class 2 CRISPR-associated endonuclease; and in some embodiments, the class 2 CRISPR-associated endonuclease is Cas9 or Cas12a.

A further aspect is for a method of reducing NRF2 expression or activity in a cell comprising introducing into the cell (a) one or more DNA sequences encoding a gRNA comprising the sequence set forth in any one of SEQ ID NO: 3-74 and (b) a nucleic acid sequence encoding a CRISPR-associated endonuclease, whereby the gRNA hybridizes to the NRF2 gene and the CRISPR-associated endonuclease cleaves the NRF2 gene, and wherein NRF2 expression or activity is reduced in the cell relative to a cell in which the one or more DNA sequences encoding the gRNA and the nucleic acid sequence encoding the CRISPR-associated endonuclease are not introduced. In some embodiments, the gRNA comprises a tracrRNA and a crRNA. In some embodiments, the gRNA is a single gRNA. In some embodiments, the CRISPR-associated endonuclease is a class 2 CRISPR-associated endonuclease; in some embodiments, the class 2 CRISPR-associated endonuclease is Cas9 or Cas12a. In some embodiments, expression of one or more allele(s) of the NRF2 gene is reduced in the cell. In some embodiments, NRF2 activity is reduced in the cell. In some embodiments, the cell is a cancer cell; in some embodiments, the cancer cell is selected from the group consisting of a lung cancer cell, a head and neck cancer cell, an esophageal cancer cell, a glioma cell, a pancreatic cancer cell, a cervical cancer cell, a breast cancer cell, a uterine cancer cell, a renal cell cancer cell, a liver cancer cell, a bladder cancer cell, a colorectal cancer cell, and a melanoma cell; in some embodiments, the lung cancer cell, head and neck cancer cell, or esophageal cancer cell is a squamous cell carcinoma cell or an adenocarcinoma cell; the glioma cell is a glioblastoma cell; or the pancreatic cancer cell is a ductal adenocarcinoma cell.

An additional aspect is for a method of reducing NRF2 expression or activity in a cell comprising introducing into the cell (a) a guide RNA (gRNA) comprising the sequence set forth in any one of SEQ ID NO: 3-74, and (b) a CRISPR-associated endonuclease, whereby the one or more gRNAs hybridize to the NRF2 gene and the CRISPR-associated endonuclease cleaves the NRF2 gene, and wherein NRF2 expression or activity is reduced in the cell relative to a cell in which the gRNA and the CRISPR-associated endonuclease are not introduced. In some embodiments, the gRNA comprises a tracrRNA and a crRNA. In some embodiments, the gRNA is a single gRNA. In some embodiments, the CRISPR-associated endonuclease is a class 2 CRISPR-associated endonuclease; and in some embodiments, the class 2 CRISPR-associated endonuclease is Cas9 or Cas12a. In some embodiments, expression of one or more allele(s) of the NRF2 gene is reduced in the cell. In some embodiments, NRF2 activity is reduced in the cell. In some embodiments, the cell is a cancer cell; in some embodiments, the cancer cell is selected from the group consisting of a lung cancer cell, a head and neck cancer cell, an esophageal cancer cell, a glioma cell, a pancreatic cancer cell, a cervical cancer cell, a breast cancer cell, a uterine cancer cell, a renal cell cancer cell, a liver cancer cell, a bladder cancer cell, a colorectal cancer cell, and a melanoma cell; and in some embodiments, the lung cancer cell, head and neck cancer cell, or esophageal cancer cell is a squamous cell carcinoma cell or an adenocarcinoma cell; the glioma cell is a glioblastoma cell; or the pancreatic cancer cell is a ductal adenocarcinoma cell.

Another aspect is for a gRNA comprising the sequence set forth in any one of SEQ ID NO: 3-74. In some embodiments, the gRNA comprises a tracrRNA and a crRNA. In some embodiments, the gRNA is a single gRNA.

A further aspect is for a pharmaceutical composition comprising the aforementioned gRNA and a pharmaceutically acceptable carrier. In some embodiments, the pharmaceutical composition further comprises a CRISPR-associated endonuclease; in some embodiments, the CRISPR-associated endonuclease is a class 2 CRISPR-associated endonuclease; and in some embodiments, the class 2 CRISPR-associated endonuclease is Cas9 or Cas12a.

An additional aspect is for an RNP complex comprising the aforementioned gRNA and a CRISPR-associated endonuclease. In some embodiments, the CRISPR-associated endonuclease is a class 2 CRISPR-associated endonuclease; and in some embodiments, the class 2 CRISPR-associated endonuclease is Cas9 or Cas12a.

Another aspect is for a pharmaceutical composition comprising the aforementioned RNP complex and a pharmaceutically acceptable carrier.

A further aspect is for a DNA sequence encoding the aforementioned gRNA or a biologically active fragment thereof. In some embodiments, the biologically active fragment is a tracrRNA or a crRNA.

An additional aspect is for a vector comprising the aforementioned DNA sequence. In some embodiments, the vector further comprises a nucleic acid sequence that encodes a CRISPR-associated endonuclease protein; in some embodiments, the CRISPR-associated endonuclease is a class 2 CRISPR-associated endonuclease; and in some embodiments, the class 2 CRISPR-associated endonuclease is Cas9 or Cas12a.

Another aspect is for a pharmaceutical composition comprising the aforementioned DNA sequence or the aforementioned vector and a pharmaceutically acceptable carrier.

A further aspect is for a pharmaceutical composition comprising the aforementioned DNA sequence, further comprising a nucleic acid sequence that encodes a CRISPR-associated endonuclease protein. In some embodiments, the CRISPR-associated endonuclease is a class 2 CRISPR-associated endonuclease; and in some embodiments, the class 2 CRISPR-associated endonuclease is Cas9 or Cas12a.

An additional aspect is for a method of treating cancer in a subject comprising administering to the subject a therapeutically effective amount of the aforementioned pharmaceutical composition. In some embodiments, the cancer is resistant to one or more chemotherapeutic agents. In some embodiments, the cancer is selected from the group consisting of lung cancer, head and neck cancer, esophageal cancer, glioma, pancreatic cancer, cervical cancer, breast cancer, uterine cancer, renal cell cancer, liver cancer, bladder cancer, colorectal cancer, and melanoma; and in some embodiments, the lung cancer, head and neck cancer, or esophageal cancer is a squamous cell carcinoma or an adenocarcinoma; the glioma is a glioblastoma; or the pancreatic cancer is a ductal adenocarcinoma. In some embodiments, the method further comprises administering one or more chemotherapeutic agents to the subject; and in some embodiments, the one or more chemotherapeutic agents are selected from the group consisting of a topoisomerase II inhibitor, a mitotic inhibitor, an alkylating agent, an antimetabolite, a topoisomerase I inhibitor, a platinum compound/complex, an immunotherapy agent, and a combination thereof. In some embodiments, the subject is a human.

Other objects and advantages will become apparent to those skilled in the art upon reference to the detailed description that hereinafter follows.

### BRIEF DESCRIPTION OF THE DRAWINGS

FIG. 1: NRF2 gene and corresponding protein domains. The image displays the structural domains of the NRF2 protein aligned to the exons of the NRF2 gene.

FIG. 2A-C: Head and Neck Cancer cells targeting exon 4 of NRF2 and assessment of chemotherapy response. (FIG. 2A) Genomic analyses of after CRISPR targeting. Genomic DNA from FaDu cells was isolated and amplified across exon 4 of the NRF2 gene. Amplicon was NGS sequenced and analyzed for indels at the CRISPR target site. Raw sequence files were aligned using the software program, CRISPResso2, to display the NRF2 allele-specific indel pattern and frameshift percent of the targeted outcomes. (FIG. 2B) Chemosensitivity Testing in response to NRF2 exon 4 targeting. Chemosensitivity was measured via CellTiter-Glo® 2.0 Assay. Targeted cells were treated with increasing concentrations of cisplatin for 72 hr. and then evaluated for cell viability. The average relative viability of cells normalized to the untreated Wt. was graphed. The error bars represent % CV. (FIG. 2C) qPCR gene expression analysis. RNA was isolated from targeted cells and converted to cDNA. Relative gene expression of NRF2 and NQO1 was measured through qPCR and analyzed with Bio-Rad CFX Maestro Software. Relative gene expression normalized to the Wt. cells was graphed.

FIG. 3A-C: Esophageal cancer cells targeting exon 4 of NRF2 and assessment of chemotherapy response. (FIG. 3A) Genomic analyses of NRF2 after CRISPR targeting. Genomic DNA from KYSE-410 cells was isolated and amplified across exon 2 and exon 4 of the NRF2 gene. Amplicon was NGS sequenced and analyzed for indels at the CRISPR target site. Raw sequence files were aligned using the software program, CRISPResso2, to display the NRF2 allele-specific indel pattern and frameshift percent of the targeted outcomes. (FIG. 3B) Chemosensitivity Testing in response to NRF2 targeting. Chemosensitivity was measured via CellTiter-Glo® 2.0 Assay. Targeted cells were treated with increasing concentrations of cisplatin for 72 hr. and then evaluated for cell viability. The average relative viability of cells normalized to the untreated Wt. was graphed. The error bars represent % CV. (FIG. 3C) qPCR gene expression analysis. RNA was isolated from targeted cells and converted to cDNA. Relative gene expression of NRF2 and NQO1 was measured through qPCR and analyzed with Bio-Rad CFX Maestro Software. Relative gene expression normalized to the Wt. cells was graphed.

FIG. 4A-C: Lung adenocarcinoma cells targeting Exon 3, 4, 5 of NRF2 and assessment of chemotherapy response. (FIG. 4A) A549 cells were transfected with each respective gRNA. Genomic DNA from each cell population was isolated and amplified across exon 3, 4 or 5 of the NRF2 gene. Amplicons were sanger sequenced and analyzed for indels at the CRISPR target site. The graph presents the total editing efficiency listed as Indel and frameshifting indel % listed as FS. (FIG. 4B) Raw sequence files from cells targeted with gRNA 76 or gRNA 83 were aligned using the software program, DECODR, to display the indel pattern (listed as INDEL and %). (FIG. 4C) The viability of wild type and gRNA 83 targeted A549 cells in response to cisplatin treatment was assessed. Viability was measured via bioreduction of MTS to a formazan product. Cells were treated with increasing concentrations of cisplatin for 72 hours then evaluated for cell viability. The average relative viability of cells in response to cisplatin is graphed above.

FIG. 5A-D: Glioblastoma cells targeting exon 4 of NRF2 and assessment of chemotherapy response. T98G cells (FIG. 5A) and LN229 cells (FIG. 5B) were transfected with each respective gRNA. Genomic DNA from each cell population was isolated and amplified across exon 4 of the NRF2 gene. Amplicons were sanger sequenced and analyzed for indels at the CRISPR target site. Raw sequence files were aligned using the software program, DECODR, to display the indel pattern (listed as INDEL and frameshift %). (FIG. 5C-D) Chemosensitivity Testing in response to NRF2 targeting. Chemosensitivity was measured via CellTiter-Glo® 2.0 Assay. Untargeted and CRISPR targeted T98G cells were treated with increasing concentrations of (FIG. 5C) temozolomide or (FIG. 5D) doxorubicin for 72 hr. and then evaluated for cell viability. The average relative viability of cells normalized to the untreated Wt. was graphed. The error bars represent % CV.

FIG. 6: Pancreatic cells targeting exon 3, 4 of NRF2. Panc-1 cells were transfected with each respective gRNA. Genomic DNA from each cell population was isolated and amplified across exon 3 or 4 of the NRF2 gene. Amplicons were sanger sequenced and analyzed for indels at the CRISPR target site. Raw sequence files were aligned using the software program, DECODR, to display the indel pattern and total gene editing efficiency (INDEL %).

FIG. 7A-B: Quantification of bioluminescence signal and Cas9 expression after localized delivery of LNPs. (FIG. 7A) Firefly luciferase LNPs were injected intratumorally in tumor-bearing xenograft mice. Twenty-four hours after LNP injection, bioluminescence imaging was performed with an IVIS Spectrum imaging system. Bioluminescence values were quantified using the Living IMAGE Software provided by Caliper (Hopkinton, MA) by measuring photon flux (photons/second) in the region of interest where bioluminescence signal emanated. (FIG. 7B) CRISPR/Cas9 LNPS were injected intratumorally in tumor-bearing xenograft mice. Seventy-two hours after injections, mice were sacrificed, and tumors were collected. RNA was isolated and converted to cDNA for Quantitative PCR. Relative gene expression of Cas9 was measured and analyzed with Bio-Rad CFX Maestro Software. Relative gene expression is normalized to PBS-injected tumors.

FIG. 8: Gene editing activity of CRISPR LNPs after localized delivery. Genomic DNA from tumors injected with respective CRISPR/Cas9 LNPs was isolated, and PCR amplified across exon 2 of the NRF2 gene. Amplicons were sanger sequenced and analyzed for indels at the CRISPR target site. Raw sequence files were aligned using the software program, DECODR, to display the indel pattern and total editing efficiency.

FIG. 9: Experimental workflow for animal based CRISPR LNP assessment. Human-derived cancer cells will be injected subcutaneously in mice. Once tumors are established, CRISPR/Cas9 LNPs will be injected intratumorally and adjuvant chemotherapy will be started. Tumor growth will be assessed by caliper measurements and overall survival will be noted.

FIG. 10A-B. NRF2 Target sites and experimental systems. (FIG. 10A) Structural domains of the NRF2 protein aligned to the exons of the NRF2 gene. Three guide RNAs were designed to cleave the NRF2 gene. Sg3 targets within the DLG motif in exon 2 while sg76, and sg83 target within the transactivation domain in exon 4. (FIG. 10B) Experimental CRISPR/Cas9 delivery workflow for targeting NRF2.

FIG. 11A-C. Genetic disruption of NRF2 at exon 2 in FaDu cells. (FIG. 11A) Genomic analyses of after CRISPR targeting. Genomic DNA from FaDu cells was isolated and amplified across exon 2 of the NRF2 gene. Amplicon was NGS sequenced and analyzed for indels at the CRISPR target site. Raw sequence files were aligned using the software program, CRISPResso2, to display the NRF2 allele-specific indel pattern and frameshift percent of the targeted outcomes. Chemosensitivity Testing in response to NRF2 exon 2 targeting; FIG. 11B—Cisplatin, FIG. 11C—5-FU. Chemosensitivity was measured via CellTiter-Glo® 2.0 Assay. Targeted cells were treated with increasing concentrations of cisplatin or 5-FU for 72 hr. and then evaluated for cell viability. The average relative viability of cells normalized to the untreated Wt. was graphed. The error bars represent % CV.

FIG. 12A-E. Genetic disruption of NRF2 at exon 4 in FaDu cells. (FIG. 12A) Genomic analyses of after CRISPR targeting. Genomic DNA from FaDu cells was isolated and amplified across exon 4 of the NRF2 gene. Amplicon was NGS sequenced and analyzed for indels at the CRISPR target site. Raw sequence files were aligned using the software program, CRISPResso2, to display the NRF2 allele-specific indel pattern and frameshift percent of the targeted outcomes. (FIG. 12B) Protein analysis by western blot. Protein was isolated from edited cells and NRF2 expression was analyzed relative to GapDH and normalized to the representative wild-type control from each transfection. (FIG. 12C) Chemosensitivity Testing in response to NRF2 exon 4 targeting. Chemosensitivity was measured via CellTiter-Glo® 2.0 Assay. Targeted cells were treated with increasing concentrations of cisplatin for 72 hr. and then evaluated for cell viability. The average relative viability of cells normalized to the untreated Wt. was graphed. The error bars represent % CV. (FIG. 12D) qPCR gene expression analysis. RNA was isolated from targeted cells and converted to cDNA. Relative gene expression of NRF2, NQO1, HMOX1 and GCLC was measured through qPCR and analyzed using the Pfaffl method. Relative gene expression normalized to the Wt. cells was graphed. (FIG. 12E) Ratio of NRF2 expression to downstream targets. Values from FIG. 12C were divided by NRF2 expression values for each condition to show the ratio of NRF2 to NQO1, HMOX1 and GCLC when NRF2 expression is reduced.

FIG. 13A-D. Genetic disruption of NRF2 in KYSE-410 cells. (FIG. 13A) Genomic analyses of NRF2 after CRISPR targeting. Genomic DNA from KYSE-410 cells was isolated and amplified across exon 2 and exon 4 of the NRF2 gene. Amplicon was NGS sequenced and analyzed for indels at the CRISPR target site. Raw sequence files were aligned using the software program, CRISPResso2, to display the NRF2 allele-specific indel pattern and frameshift percent of the targeted outcomes. (FIG. 13B) Chemosensitivity Testing in response to NRF2 targeting. Chemosensitivity was measured via CellTiter-Glo® 2.0 Assay. Targeted cells were treated with increasing concentrations of cisplatin for 72 hr. and then evaluated for cell viability. The average relative viability of cells normalized to the untreated Wt. was graphed. The error bars represent % CV. (FIG. 13C) qPCR gene expression analysis. RNA was isolated from targeted cells and converted to cDNA. Relative gene expression of NRF2, NQO1, HMOX1, and GCLC was measured through qPCR and analyzed with the Pfaffl method. Relative gene expression normalized to the Wt. cells was graphed. (FIG. 13D) Ratio of NRF2 expression to downstream targets. Values from FIG. 13C were divided by NRF2 expression values for each condition to show the ratio of NRF2 to NQO1, HMOX1 and GCLC when NRF2 expression is reduced.

FIG. 14A-B. Exon skipping NRF2 gene after CRISPR/Cas9 disruption. cDNA from cells targeted with a CRISPR guide RNA in exon 2 or exon 4 was collected at 72 hr post-transfection and was used as the template for PCR amplification with primers that spanned from the 5′UTR region through Exon 5 (798 bp) of the NRF2 gene. (FIG. 14A) A diagram to show targeting of Exon 2 and Exon 4, and the resulting Exon skip variants. (FIG. 14B) cDNA gels for each sgRNA targeting condition with exon skip variants listed beneath.

FIG. 15A-G. Sustained genetic and functional disruption of NRF2 over time. (FIG. 15A) Experimental workflow to assess editing outcomes in NRF2 2 weeks after transfection. (FIG. 15B) Genomic analyses of NRF2 2 weeks post CRISPR/Cas9 targeting. Genomic DNA from FaDu cells was isolated and amplified across exon 2 and exon 4 of the NRF2 gene 13 days after transfection. Amplicon was NGS sequenced and analyzed for indels at the CRISPR target site. Raw sequence files were aligned using the software program, CRISPResso2, to display the NRF2 allele-specific indel pattern and (FIG. 15D) frameshift percent of the targeted outcomes. (FIG. 15C) Sustained Chemosensitivity Testing 2 weeks after CRISPR/Cas9 targeting of NRF2. Chemosensitivity was measured via CellTiter-Glo® 2.0 Assay. Targeted cells that were maintained in culture for 2 weeks post-transfection was treated with increasing concentrations of cisplatin for 72 hr. and then evaluated for cell viability. The average relative viability of cells normalized to the untreated Wt. was graphed. The error bars represent % CV. (FIG. 15E) qPCR gene expression analysis. RNA was isolated from edited cells at the 2 week collection timepoint, converted to cDNA and transcript levels of NRF2, NQO1, HMOX1 and GCLC were measured by qPCR. (FIG. 15F) Ratio of NRF2 expression to downstream targets. Values from FIG. 15E were divided by NRF2 expression values for each condition to show the ratio of NRF2 to NQO1, HMOX1 and GCLC when NRF2 expression is reduced 2 weeks post-targeting. (FIG. 15G) Exon skipping 2 weeks post targeting. cDNA from cells targeted with a CRISPR guide RNA in exon 2 or exon 4 was collected at 2 weeks post-transfection and was used as the template for PCR amplification with primers that spanned from the 5′UTR region through Exon 5 (798 bp) of the NRF2 gene. cDNA gel for each condition two weeks post targeting is shown.

FIG. 16A-B. NRF2 impacts tumor cell development. (FIG. 16A) Illustrates downstream pathways regulated by the NRF2-KEAP1 pathway. (FIG. 16B) shows NRF2 levels under normal cell conditions, conditions of cellular stress, and where CRISPR knockdown via gene disruption has diminished NRF2 levels.

FIG. 17. The NRF2 gene with associated sgRNAs. Target sites in the NRF2 gene are indicated by arrows. Each sgRNA represents the region in which a double stranded break within the DNA is created.

FIG. 18A-E. Workflow of genotype analysis of gene editing efficiency (FIG. 18A) Sanger sequencing workflow used to analyze the indel spectrum generated by the sgRNA editing, along with the editing efficiencies. (FIG. 18B and FIG. 18C) illustrate the editing efficiencies and indel spectrums of exon 3 (sgRNA5) and exon 4 (sgRNA 83) respectively; (FIG. 18D) shows the experimental distribution of indels with each dot representing a single gene editing experiment; (FIG. 18E) shows the mutational outcome of the editing efficiencies represented in FIG. 18C; each dot represents an individual gene editing experiment and its reflected gene disruption profile.

FIG. 19A-C. Workflow of functional outcomes of gene editing (FIG. 19A) shows the workflow from transfection of cells to downstream phenotypic assays; (FIG. 19B) shows the restoration of chemosensitivity for edited populations compared to unedited populations; (FIG. 19C) shows protein analysis by western blot. Protein was isolated from edited cells and NRF2 expression was analyzed relative to GapDH and normalized to the representative wild-type control from each transfection.

FIG. 20A-B. (FIG. 20A) Genomic analyses of spCas9-13 NRF2 knockout clones in A549 cells. A549 cells transfected with spCas9-13 gRNA were Sanger sequenced and analyzed for indel activity by DECODR. (FIG. 20B) Sequence data from DECODR for spCas9-13 gRNA in A549 cells.

FIG. 21A-B. (FIG. 21A) Genomic analyses of gRNA76 NRF2 knockout clones in A549 cells. A549 cells transfected with gRNA76 were Sanger sequenced and analyzed for indel activity by DECODR. (FIG. 21B) Sequence data from DECODR for gRNA76 in A549 cells.

FIG. 22A-B. (FIG. 22A) Genomic analyses of spCas9-21 NRF2 knockout clones in A549 cells. A549 cells transfected with spCas9-21 gRNA were Sanger sequenced and analyzed for indel activity by DECODR. (FIG. 22B) Sequence data from DECODR for spCas9-21 gRNA in A549 cells.

FIG. 23A-B. (FIG. 23A) Genomic analyses of gRNA83 NRF2 knockout clones in A549 cells. A549 cells transfected with gRNA83 were Sanger sequenced and analyzed for indel activity by DECODR. (FIG. 23B) Sequence data from DECODR for gRNA83 in A549 cells.

FIG. 24A-B. (FIG. 24A) Genomic analyses of spCas9-25 NRF2 knockout clones in A549 cells. A549 cells transfected with spCas9-25 gRNA were Sanger sequenced and analyzed for indel activity by DECODR. (FIG. 24B) Sequence data from DECODR for spCas9-25 gRNA in A549 cells.

FIG. 25A-B. (FIG. 25A) Genomic analyses of spCas9-26 NRF2 knockout clones in A549 cells. A549 cells transfected with spCas9-26 gRNA were Sanger sequenced and analyzed for indel activity by DECODR. (FIG. 25B) Sequence data from DECODR for spCas9-26gRNA in A549 cells.

FIG. 26A-B. (FIG. 26A) Genomic analyses of gRNA76 NRF2 knockout clones in H1703 cells. H1703 cells transfected with gRNA76 were Sanger sequenced and analyzed for indel activity by DECODR. (FIG. 26B) Sequence data from DECODR for gRNA76 in H1703 cells.

FIG. 27A-B. (FIG. 27A) Genomic analyses of spCas9-21 NRF2 knockout clones in H1703 cells. H1703 cells transfected with spCas9-21 gRNA were Sanger sequenced and analyzed for indel activity by DECODR. (FIG. 27B) Sequence data from DECODR for spCas9-21 gRNA in H1703 cells.

FIG. 28A-B. (FIG. 26A) Genomic analyses of gRNA83 NRF2 knockout clones in H1703 cells. H1703 cells transfected with gRNA83 were Sanger sequenced and analyzed for indel activity by DECODR. (FIG. 26B) Sequence data from DECODR for gRNA83 in H1703 cells.

FIG. 29A-B. (FIG. 29A) Genomic analyses of spCas9-25 NRF2 knockout clones in H1703 cells. H1703 cells transfected with spCas9-25 gRNA were Sanger sequenced and analyzed for indel activity by DECODR. (FIG. 29B) Sequence data from DECODR for spCas9-25 gRNA in H1703 cells.

FIG. 30A-B. (FIG. 30A) Genomic analyses of spCas9-26 NRF2 knockout clones in H1703 cells. H1703 cells transfected with spCas9-26 gRNA were Sanger sequenced and analyzed for indel activity by DECODR. (FIG. 30B) Sequence data from DECODR for spCas9-26 gRNA in H1703 cells.

### DETAILED DESCRIPTION

Applicant has solved the stated problem.

CRISPR/Cas9 mediated precision targeting of genes that enable drug resistance or accelerate tumor growth is an ideal intervention to render genes responsible for resistance inactive and stem the production of proteins that protect tumor cells from treatment. Applicant herein uses a CRISPR/Cas9 gene-editing tool to disable the NRF2 (Nuclear Factor Erythroid 2-Related Factor) gene, rendering it incapable of producing a functional protein that protects the anti-cancer therapies. Cells with this gene knockout will be more sensitive to chemotherapeutic agents, in fact all forms of therapy including radiation. NRF2 expression, a master regulator involved in cellular responses to oxidative and/or electrophilic stress, is elevated dramatically during the process of tumor the genesis and is an upstream regulator of processes that account for enhanced resistance of cancer cells to chemotherapeutic drugs (Zhao, J. et al. Nrf2 Mediates Metabolic Reprogramming in Non-Small Cell Lung Cancer. Front Oncol 10, (2020); Bialk, P., Wang, Y., Banas, K. & Kmiec, E. B. Functional Gene Knockout of NRF2 Increases Chemosensitivity of Human Lung Cancer A549 Cells in Vitro and in a Xenograft Mouse Model. Mol Ther Oncolytics 11, 75-89 (2018)). Hence, Applicant's treatment strategy can combine CRISPR directed gene editing with traditional chemotherapy, but it is also can be a pathway with a combination of gene editing with other forms of cancer therapy.

### Definitions

Applicant specifically incorporates the entire contents of all cited references in this disclosure. Further, when an amount, concentration, or other value or parameter is given as either a range or a list of upper values and lower values, this is to be understood as specifically disclosing all ranges formed from any pair of any upper range limit or value and any lower range limit or value, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

The indefinite articles “a” and “an”, as used herein in the specification and in the claims, unless clearly indicated to the contrary, should be understood to mean “at least one”.

The phrase “and/or”, as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, “either”, “one of”, “only one of”, “exactly one of”. “Consisting essentially of”, when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

A “Clustered Regularly Interspaced Short Palindromic Repeat (CRISPR)-associated endonuclease protein-binding domain” or “Cas binding domain” refers to a nucleic acid element or domain within a nucleic acid sequence or polynucleotide sequence that, in an effective amount, will bind or have an affinity for one or a plurality of CRISPR-associated endonuclease (or functional fragments thereof). In some embodiments, in the presence of the one or a plurality of proteins (or functional fragments thereof) and a target sequence, the one or plurality of proteins and the nucleic acid element forms a biologically active CRISPR complex and/or can be enzymatically active on a target sequence. In some embodiments, the CRISPR-associated endonuclease is a class 1 or class 2 CRISPR-associated endonuclease, and in some embodiments, a Cas9 or Cas12a endonuclease. The Cas9 endonuclease can have a nucleotide sequence identical to the wild type Streptococcus pyogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Such species include: Acidovorax avenae, Actinobacillus pleuropneumoniae, Actinobacillus succinogenes, Actinobacillus suis, Actinomyces sp., Cycliphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., and Verminephrobacter eiseniae (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas9 endonucleases). In some embodiments, the CRISPR-associated endonuclease can be a Cas12a nuclease. The Cas12a nuclease can have a nucleotide sequence identical to a wild type Prevotella or Francisella sequence (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas12 endonucleases).

In some embodiments, the terms “(CRISPR)-associated endonuclease protein-binding domain” or “Cas binding domain” refer to a nucleic acid element or domain (e.g. and RNA element or domain) within a nucleic acid sequence that, in an effective amount, will bind to or have an affinity for one or a plurality of CRISPR-associated endonucleases (or functional fragments or variants thereof that are at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to a CRISPR-associated endonucleas). In some embodiments, the Cas binding domain consists of at least or no more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially associates or binds to a biologically active CRISPR-associated endonuclease at a concentration and within a microenvironment suitable for CRISPR system formation.

The “Clustered Regularly Interspaced Short Palindromic Repeats (CRISPR)—CRISPR associated (Cas) (CRISPR-Cas) system guide RNA” or “CRISPR-Cas system guide RNA” may comprise a transcription terminator domain. The term “transcription terminator domain” refers to a nucleic acid element or domain within a nucleic acid sequence (or polynucleotide sequence) that, in an effective amount, prevents bacterial transcription when the CRISPR complex is in a bacterial species and/or creates a secondary structure that stabilizes the association of the nucleic acid sequence to one or a plurality of Cas proteins (or functional fragments thereof) such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of at least or no more than about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially drives association of the nucleic acid sequence (sgRNA, crRNA with tracrRNA, or other nucleic acid sequence) to a biologically active CRISPR complex at a concentration and microenvironment suitable for CRISPR complex formation.

The term “DNA-binding domain” refers to a nucleic acid element or domain within a nucleic acid sequence (e.g. a guide RNA) that is complementary to NRF2. In some embodiments, the DNA-binding domain will bind or have an affinity for an NRF2 gene such that, in the presence of a biologically active CRISPR complex, one or plurality of Cas proteins can be enzymatically active on the target sequence. In some embodiments, the DNA binding domain comprises at least one sequence that is capable of forming Watson Crick basepairs with a target sequence as part of a biologically active CRISPR system at a concentration and microenvironment suitable for CRISPR system formation.

“CRISPR system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is an guanine and N is any naturally occurring nucleic acid. In some embodiments the target DNA comprises any one or combination of NNG, NNA, GAA, NNAGAAW, NGGNG, and TTTV, where G is an guanine, A is adenine, T is thymine, N is any naturally occurring nucleic acid, and V is guanine, cytosine, or adenine.

In some embodiments, a target sequence is located in the nucleus or cytoplasm of a cell.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a class 2 Cas9 or Cas12a protein. In some embodiments, the chemically synthesized guide sequence is used in conjunction with one or more vectors, wherein each vector comprises a coding sequence that encodes a CRISPR enzyme, such as a class 2 Cas9 or Cas12a protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, one or more additional guide sequence, tracr mate sequence, and tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid for a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

In some embodiments, a guide RNA can be a short, synthetic, chimeric tracrRNA/crRNA (a “single-guide RNA” or “sgRNA”). A guide RNA may also comprise two short, synthetic tracrRNA/crRNAs (a “dual-guide RNA” or ‘dgRNA”).

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features.

As used herein, “cancer” refers to all types of cancer or neoplasm or malignant tumors found in humans, including, but not limited to: leukemias, lymphomas, melanomas, carcinomas and sarcomas. As used herein, the terms or language “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells. In certain embodiments, the cancer is a blood tumor (i.e., a non-solid tumor). In some embodiments, the cancer is lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, pancreatic ductal adenocarcinoma, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sezary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor (see, e.g., Kerins et al., Sci. Rep. 8:12846 (2018)).

In certain embodiments, the cancer is a solid tumor. A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Specific criteria for the staging of cancer are dependent on the specific cancer type based on tumor size, histological characteristics, tumor markers, and other criteria known by those of skill in the art. Generally, cancer stages can be described as follows:
Stage 0—Carcinoma in situ
Stage I, Stage II, and Stage III—Higher numbers indicate more extensive disease: Larger tumor size and/or spread of the cancer beyond the organ in which it first developed to nearby lymph nodes and/or tissues or organs adjacent to the location of the primary tumor
Stage IV—The cancer has spread to distant tissues or organs

As used herein, a “variant”, “mutant”, or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, the terms “treat,” “treating” or “treatment” refer to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the QOL as judged by a qualified individual, e.g., a treating physician, e.g., using accepted assessment tools of pain and QOL is not considered to be a “treatment” of the cancer.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

A “cancer that is resistant to one or more chemotherapeutic agents” is a cancer that does not respond, or ceases to respond to treatment with a chemotherapeutic regimen, i.e., does not achieve at least stable disease (i.e., stable disease, partial response, or complete response) in the target lesion either during or after completion of the chemotherapeutic regimen. Resistance to one or more chemotherapeutic agents results in, e.g., tumor growth, increased tumor burden, and/or tumor metastasis.

A “therapeutically effective amount” is that amount sufficient, at dosages and for periods of time necessary, to achieve a desired therapeutic result, such as for treatment of a disease (e.g. cancer), condition, or disorder, and/or pharmacokinetic or pharmacodynamic effect of the treatment in a subject. A therapeutically effective amount can be administered in one or more administrations. The therapeutically effective amount may vary according to factors such as the disease state, age, sex, and weight of the subject.

### CRISPR/Endonucleases

CRISPR/endonuclease (e.g., CRISPR/Cas9) systems are known in the art and are described, for example, in U.S. Pat. No. 9,925,248, which is incorporated by reference herein in its entirety. CRISPR-directed gene editing can identify and execute DNA cleavage at specific sites within the chromosome at a surprisingly high efficiency and precision. The natural activity of CRISPR/Cas9 is to disable a viral genome infecting a bacterial cell. Subsequent genetic reengineering of CRISPR/Cas function in human cells presents the possibility of disabling human genes at a significant frequency.

In bacteria, the CRISPR/Cas loci encode RNA-guided adaptive immune systems against mobile genetic elements (viruses, transposable elements and conjugative plasmids). Three types (I-Ill) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA) containing a DNA binding region (spacer) which is complementary to the target gene. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called a spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM).

The compositions described herein can include a nucleic acid encoding a CRISPR-associated endonuclease. The CRISPR-associated endonuclease can be, e.g., a class 1 CRISPR-associated endonuclease or a class 2 CRISPR-associated endonuclease. Class 1 CRISPR-associated endonucleases include type I, type III, and type IV CRISPR-Cas systems, which have effector molecules that comprise multiple subunits. For class 1 CRISPR-associated endonucleases, effector molecules can include, in some embodiments, Cas7 and Cas5, along with, in some embodiments, SS (Cas11) and Cas8a1; Cas8b1; Cas8c; Cas8u2 and Cas6; Cas3″ and Cas10d; Cas SS (Cas11), Cas8e, and Cas6; Cas8f and Cas6f; Cas6f; Cas8-like (Csf1); SS (Cas11) and Cas8-like (Csf1); or SS (Cas11) and Cas10. Class 1 CRISPR-associated endonucleases also be associated with, in some embodiments, target cleavage molecules, which can be Cas3 (type 1) or Cas10 (type Ill) and spacer acquisition molecules such as, e.g., Cas1, Cas2, and/or Cas4. See, e.g., Koonin et al., Curr. Opin. Microbiol. 37:67-78 (2017); Strich & Chertow, J. Clin. Microbiol. 57:1307-18 (2019).

Class 2 CRISPR-associated endonucleases include type I, type V, and type VI CRISPR-Cas systems, which have a single effector molecule. For class 2 CRISPR-associated endonucleases, effector molecules can include, in some embodiments, Cas9, Cas12a (cpf1), Cas12b1 (c2c1), Cas12a2, Cas12b2, Cas12c (c2c3), Cas12d (CasY), Cas12e (CasX), Cas12f1 (Cas14a), Cas12f2 (Cas14b), Cas12f3 (Cas14c), Cas12g, Cas12h, Cas12i, Cas12j (Casϕ), Cas12k (c2c5), Cas13a (c2c2), Cas13b1 (c2c6), Cas13b2 (c2c6), Cas13bt, Cas13c (c2c7), Cas13ct, Cas13d, Cas13X, Cas13Y, c2c4, c2c8, c2c9, and/or c2c10. See, e.g., Koonin et al., Curr. Opin. Microbiol. 37:67-78 (2017); Strich & Chertow, J. Clin. Microbiol. 57:1307-18 (2019); Makarova et al., Nat. Rev. Microbiol. 18:67-83 (2020); Pausch et al., Science 369:333-37 (2020); Tong et al., Cell Dev. Biol. 8:622103 (2021); Xu et al., Nat. Meth. 18:499-506 (2021); Kannan et al., Nat. Biotechnol. 40:194-97 (2022); Liu et al., Mol. Cell 82:333-47 (2022).

In some embodiments, the CRISPR-associated endonuclease can be a Cas9 nuclease. The Cas9 nuclease can have a nucleotide sequence identical to the wild type Streptococcus pyogenes sequence. In some embodiments, the CRISPR-associated endonuclease can be a sequence from other species, for example other Streptococcus species, such as agalactiae, anginosis, canis, castoreus, constella, constellatus, denstasini, devriesei, dysgalactiae, equi, equinus, gallolyticus, infantarius, iniae, lutetiensis, macacae, massiliensis, mitis, mutans, ovis, parasanguinis, parauberis, phocae, pseudoporcinus, plurextorum, ratti, sanguinis, sobrinus, suis, thermophilus, or tigurinus; Pseudomona aeruginosa, Escherichia coli, or other sequenced bacteria genomes and archaea; or other prokaryotic microorganisms. Such species include: Acidaminoccus sp., Acidovorax avenae, Actinobacillus pleuropneumoniae, Actinobacillus succinogenes, Actinobacillus suis, Actinomyces sp., Alicyclobacillus acidiphilus, Alicyclobacillus acidoterrestris, Aminomonas paucivorans, Bacillus cereus, Bacillus hisahsii, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Cycliphilus denitrificans, Dinoroseobacter shibae, Dolosigranulum pigrum, Enterococcus durans, Enterococcus faecalis, Enterococcus faecium, Enterococcus hirae, Enterococcus italicus, Enterococcus mundtii, Enterococcus phoeniculicola, Enterococcus villorum, Eubacterium dolichum, Francisella novicida, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lachnospiraceae bacterium, Lactobacillus apodemi, Lactobacillus animalis, Lactobacillus crispatus, Leptotrichia shahii, Listeria innocua, Listeria selligeri, Listeria ivanovii, Listeria monocytogenes, Listeriaceae bacterium, Methylocystis sp., Methylosinus trichosporium, Mobiluncus mulieris, Moraxella bovoculi, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Prevotella bryantii, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus auricularis, Staphylococcus lugdunensis, Streptococcus canis, Streptococcus sp., Subdoligranulum sp., Sulfuricurvum sp., Tistrella mobilis, Treponema sp., and Verminephrobacter eiseniae.

Alternatively, the wild type Streptococcus pyogenes Cas9 sequence can be modified. The nucleic acid sequence can be codon optimized for efficient expression in mammalian cells, e.g., human cells. A Cas9 nuclease sequence codon optimized for expression in human cells sequence can be for example, the Cas9 nuclease sequence encoded by any of the expression vectors listed in Genbank accession numbers NZ_LS483338.1 GI:69900935, KM099231.1 GI:669193757; KM099232.1 GI:669193761; or KM099233.1 GI:669193765. Alternatively, the Cas9 nuclease sequence can be, for example, the sequence contained within a commercially available vector such as pX458, pX330 or pX260 from Addgene (Cambridge, Mass.). In some embodiments, the Cas9 endonuclease can have an amino acid sequence that is a variant or a fragment of any of the Cas9 endonuclease sequences of Genbank accession numbers NZ_LS483338.1 GI:69900935, KM099231.1 GI:669193757; KM099232.1 GI:669193761; or KM099233.1 GI:669193765 or Cas9 amino acid sequence of pX458, pX330 or pX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas9 polypeptide. See, e.g., US2019/0032036, US2023/0075913, US2023/0031899, US2023/0021641, US2022/0307001, US2022/0235340, US2022/0204954, US2022/0154158, US2022/0154157, US2021/0301269, US2021/0284978, US2021/0261932, US2021/0163907, US2021/0147861, US2020/0332271, US2020/0318086, US20200299657, US2020/0277586, US2020/0199552; each of which incorporated by reference herein in its entirety.

In some embodiments, the CRISPR-associated endonuclease can be a Cas12a nuclease. The Cas12a nuclease can have a nucleotide sequence identical to a wild type Prevotella or Francisella sequence. Alternatively, a wild type Prevotella or Francisella Cas12a sequence can be modified. The nucleic acid sequence can be codon optimized for efficient expression in mammalian cells, e.g., human cells. A Cas12a nuclease sequence codon optimized for expression in human cells sequence can be for example, the Cas12a nuclease sequence encoded by any of the expression vectors listed in Genbank accession numbers NZ_CP010070.1 GI: 24818655, M F193599.1 GI: 1214941796, KY985374.1 GI: 1242863785, KY985375.1 GI: 1242863787, or KY985376.1 GI: 1242863789. Alternatively, the Cas12a nuclease sequence can be, for example, the sequence contained within a commercially available vector such as pAs-Cpf1 or pLb-Cpf1 from Addgene (Cambridge, Mass.). In some embodiments, the Cas12a endonuclease can have an amino acid sequence that is a variant or a fragment of any of the Cas12a endonuclease sequences of Genbank accession numbers NZ_CP010070.1 GI: 24818655, MF193599.1 GI: 1214941796, KY985374.1 GI: 1242863785, KY985375.1 GI: 1242863787, or KY985376.1 GI: 1242863789 or Cas12a amino acid sequence of pAs-Cpf1 or pLb-Cpf1 (Addgene, Cambridge, Mass.). The Cas12a nucleotide sequence can be modified to encode biologically active variants of Cas12a, and these variants can have or can include, for example, an amino acid sequence that differs from a wild type Cas12a by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas12a polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas12a polypeptide. See, e.g., US2019/0233814, US2019/0264186, US2023/0040148, US2021/0348144, US2021/0309701, US2021/0230567, US2021/0155911, US2020/0263190, US2020/0216825, US2021/0115421, US2021/0079366, US2020/0255861, US2019/0010481; each of which incorporated by reference herein in its entirety.

The compositions described herein may also include sequence encoding a gRNA comprising a DNA-binding domain that is complementary to a target domain from an NRF2 gene, and a CRISPR-associated endonuclease protein-binding domain. In some embodiments. The guide RNA sequence can be a sense or anti-sense sequence. The guide RNA sequence may include a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. The specific sequence of the guide RNA may vary, but, regardless of the sequence, useful guide RNA sequences will be those that minimize off-target effects while achieving high efficiency. In some embodiments, the guide RNA sequence achieves complete ablation of the NRF2 gene. In some embodiments, the guide RNA sequence achieves complete ablation of a variant NRF2 gene without affecting expression or activity of a wild-type NRF2 gene.

In some embodiments, the DNA-binding domain varies in length from about 20 to about 55 nucleotides, for example, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides. In some embodiments, the Cas protein-binding domain is from about 30 to about 55 nucleotides in length, for example, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides.

In some embodiments, the compositions comprise one or more nucleic acid (i.e. DNA) sequences encoding the guide RNA and the CRISPR endonuclease. When the compositions are administered as a nucleic acid or are contained within an expression vector, the CRISPR endonuclease can be encoded by the same nucleic acid or vector as the guide RNA sequence. In some embodiments, the CRISPR endonuclease can be encoded in a physically separate nucleic acid from the guide RNA sequence or in a separate vector. The nucleic acid sequence encoding the guide RNA may comprise a DNA binding domain, a Cas protein binding domain, and a transcription terminator domain.

The nucleic acid encoding the guide RNA and/or the CRISPR endonuclease may be an isolated nucleic acid. An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids also can be obtained by mutagenesis of, e.g., a naturally occurring portion of a Cas9-encoding DNA (in accordance with, for example, the formula above).

Recombinant constructs are also provided herein and can be used to transform cells in order to express the CRISPR endonuclease and/or a guide RNA complementary to an NRF2 gene (in some embodiments, a variant NRF2 gene found only in cancer cells). A recombinant nucleic acid construct may comprise a nucleic acid encoding a CRISPR endonuclease and/or a guide RNA complementary to an NRF2 gene (in some embodiments, a variant NRF2 gene found only in cancer cells), operably linked to a promoter suitable for expressing the CRISPR endonuclease and/or a guide RNA complementary to the NRF2 gene (in some embodiments, the variant NRF2 gene) in the cell. In some embodiments the nucleic acid encoding a CRISPR endonuclease is operably linked to the same promoter as the nucleic acid encoding the guide RNA. In other embodiments, the nucleic acid encoding a CRISPR endonuclease and the nucleic acid encoding the guide RNA are operably linked to different promoters. In some embodiments, the nucleic acid encoding a CRISPR endonuclease and/or the nucleic acid encoding a guide RNA are operably linked to a lung specific promoter.

In some embodiments, one or more CRISPR endonucleases and one or more guide RNAs may be provided in combination in the form of ribonucleoprotein particles (RNPs). An RNP complex can be introduced into a subject by means of, e.g., injection, electroporation, nanoparticles, vesicles, and/or with the assistance of cell-penetrating peptides.

DNA vectors containing nucleic acids such as those described herein also are also provided. A “DNA vector” is a replicon, such as a plasmid, phage, or cosmid, into which another DNA segment may be inserted so as to bring about the replication of the inserted segment. Generally, a DNA vector is capable of replication when associated with the proper control elements. Suitable vector backbones include, for example, those routinely used in the art such as plasmids, viruses, artificial chromosomes, BACs, YACs, or PACs. The term “DNA vector” includes cloning and expression vectors, as well as viral vectors and integrating vectors. An “expression vector” is a vector that includes a regulatory region. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The DNA vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The DNA vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region (e.g. a promoter) and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Direct injection of adenoviral vectors into lung tumors has been a routine procedure in clinical trials evaluating gene therapy of lung cancer. Dong et al., J. Int. Med. Res. 36, 1273-1287 (2008); Li et al., Cancer Gene Ther. 20, 251-259 (2013); Zhou et al., Cancer Gene Ther. 23, 1-6 (2016). Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al., BioTechniques, 34:167-71 (2003). A large variety of such vectors are known in the art and are generally available.

Suitable nucleic acid delivery systems include recombinant viral vector, typically sequence from at least one of an Ad, AAV, helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, in some embodiments about one polynucleotide. In embodiments in which the polynucleotide is to be administered with a non-viral vector, use of between from about 0.1 ng to about 4000 μg will often be useful e.g., about 0.1 ng to about 3900 μg, about 0.1 ng to about 3800 μg, about 0.1 ng to about 3700 μg, about 0.1 ng to about 3600 μg, about 0.1 ng to about 3500 μg, about 0.1 ng to about 3400 μg, about 0.1 ng to about 3300 μg, about 0.1 ng to about 3200 μg, about 0.1 ng to about 3100 μg, about 0.1 ng to about 3000 μg, about 0.1 ng to about 2900 μg, about 0.1 ng to about 2800 μg, about 0.1 ng to about 2700 μg, about 0.1 ng to about 2600 μg, about 0.1 ng to about 2500 μg, about 0.1 ng to about 2400 μg, about 0.1 ng to about 2300 μg, about 0.1 ng to about 2200 μg, about 0.1 ng to about 2100 μg, about 0.1 ng to about 2000 μg, about 0.1 ng to about 1900 μg, about 0.1 ng to about 1800 μg, about 0.1 ng to about 1700 μg, about 0.1 ng to about 1600 μg, about 0.1 ng to about 1500 μg, about 0.1 ng to about 1400 μg, about 0.1 ng to about 1300 μg, about 0.1 ng to about 1200 μg, about 0.1 ng to about 1100 μg, about 0.1 ng to about 1000 μg, about 0.1 ng to about 900 μg, about 0.1 ng to about 800 μg, about 0.1 ng to about 700 μg, about 0.1 ng to about 600 μg, about 0.1 ng to about 500 μg, about 0.1 ng to about 400 μg, about 0.1 ng to about 300 μg, about 0.1 ng to about 200 μg, about 0.1 ng to about 100 μg, about 0.1 ng to about 90 μg, about 0.1 ng to about 80 μg, about 0.1 ng to about 70 μg, about 0.1 ng to about 60 μg, about 0.1 ng to about 50 μg, about 0.1 ng to about 40 μg, about 0.1 ng to about 30 μg, about 0.1 ng to about 20 μg, about 0.1 ng to about 10 μg, about 0.1 ng to about 1 μg, about 0.1 ng to about 900 ng, about 0.1 ng to about 800 ng, about 0.1 ng to about 700 ng, about 0.1 ng to about 600 ng, about 0.1 ng to about 500 ng, about 0.1 ng to about 400 ng, about 0.1 ng to about 300 ng, about 0.1 ng to about 200 ng, about 0.1 ng to about 100 ng, about 0.1 ng to about 90 ng, about 0.1 ng to about 80 ng, about 0.1 ng to about 70 ng, about 0.1 ng to about 60 ng, about 0.1 ng to about 50 ng, about 0.1 ng to about 40 ng, about 0.1 ng to about 30 ng, about 0.1 ng to about 20 ng, about 0.1 ng to about 10 ng, about 0.1 ng to about 1 ng, about 1 ng to about 4000 μg, about 1 ng to about 3900 μg, about 1 ng to about 3800 μg, about 1 ng to about 3700 μg, about 1 ng to about 3600 μg, about 1 ng to about 3500 μg, about 1 ng to about 3400 μg, about 1 ng to about 3300 μg, about 1 ng to about 3200 μg, about 1 ng to about 3100 μg, about 1 ng to about 3000 μg, about 1 ng to about 2900 μg, about 1 ng to about 2800 μg, about 1 ng to about 2700 μg, about 1 ng to about 2600 μg, about 1 ng to about 2500 μg, about 1 ng to about 2400 μg, about 1 ng to about 2300 μg, about 1 ng to about 2200 μg, about 1 ng to about 2100 μg, about 1 ng to about 2000 μg, about 1 ng to about 1900 μg, about 1 ng to about 1800 μg, about 1 ng to about 1700 μg, about 1 ng to about 1600 μg, about 1 ng to about 1500 μg, about 1 ng to about 1400 μg, about 1 ng to about 1300 μg, about 1 ng to about 1200 μg, about 1 ng to about 1100 μg, about 1 ng to about 1000 μg, about 1 ng to about 900 μg, about 1 ng to about 800 μg, about 1 ng to about 700 μg, about 1 ng to about 600 μg, about 1 ng to about 500 μg, about 1 ng to about 400 μg, about 1 ng to about 300 μg, about 1 ng to about 200 μg, about 1 ng to about 100 μg, about 1 ng to about 90 μg, about 1 ng to about 80 μg, about 1 ng to about 70 μg, about 1 ng to about 60 μg, about 1 ng to about 50 μg, about 1 ng to about 40 μg, about 1 ng to about 30 μg, about 1 ng to about 20 μg, about 1 ng to about 10 μg, about 1 ng to about 1 μg, about 1 ng to about 900 ng, about 1 ng to about 800 ng, about 1 ng to about 700 ng, about 1 ng to about 600 ng, about 1 ng to about 500 ng, about 1 ng to about 400 ng, about 1 ng to about 300 ng, about 1 ng to about 200 ng, about 1 ng to about 100 ng, about 1 ng to about 90 ng, about 1 ng to about 80 ng, about 1 ng to about 70 ng, about 1 ng to about 60 ng, about 1 ng to about 50 ng, about 1 ng to about 40 ng, about 1 ng to about 30 ng, about 1 ng to about 20 ng, about 1 ng to about 10 ng, about 10 ng to about 4000 μg, about 20 ng to about 4000 μg, about 30 ng to about 4000 μg, about 40 ng to about 4000 μg, about 50 ng to about 4000 μg, about 60 ng to about 4000 μg, about 70 ng to about 4000 μg, about 80 ng to about 4000 μg, about 90 ng to about 4000 μg, about 100 ng to about 4000 μg, about 200 ng to about 4000 μg, about 300 ng to about 4000 μg, about 400 ng to about 4000 μg, about 500 ng to about 4000 μg, about 600 ng to about 4000 μg, about 700 ng to about 4000 μg, about 800 ng to about 4000 μg, about 900 ng to about 4000 μg, about 1 μg to about 4000 μg, 10 μg to about 4000 μg, 20 μg to about 4000 μg, 30 μg to about 4000 μg, 40 μg to about 4000 μg, 50 μg to about 4000 μg, 60 μg to about 4000 μg, 70 μg to about 4000 μg, 80 μg to about 4000 μg, 90 μg to about 4000 μg, 100 μg to about 4000 μg, 200 μg to about 4000 μg, 300 μg to about 4000 μg, 400 μg to about 4000 μg, 500 μg to about 4000 μg, 600 μg to about 4000 μg, 700 μg to about 4000 μg, 800 μg to about 4000 μg, 900 μg to about 4000 μg, 1000 μg to about 4000 μg, 1100 μg to about 4000 μg, 1200 μg to about 4000 μg, 1300 μg to about 4000 μg, 1400 μg to about 4000 μg, 1500 μg to about 4000 μg, 1600 μg to about 4000 μg, 1700 μg to about 4000 μg, 1800 μg to about 4000 μg, 1900 μg to about 4000 μg, 2000 μg to about 4000 μg, 2100 μg to about 4000 μg, 2200 μg to about 4000 μg, 2300 μg to about 4000 μg, 2400 μg to about 4000 μg, 2500 μg to about 4000 μg, 2600 μg to about 4000 μg, 2700 μg to about 4000 μg, 2800 μg to about 4000 μg, 2900 μg to about 4000 μg, 3000 μg to about 4000 μg, 3100 μg to about 4000 μg, 3200 μg to about 4000 μg, 3300 μg to about 4000 μg, 3400 μg to about 4000 μg, 3500 μg to about 4000 μg, 3600 μg to about 4000 μg, 3700 μg to about 4000 μg, 3800 μg to about 4000 μg, or 3900 μg to about 4000 μg.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector (Geller et al., J. Neurochem 64:487 (1995); Lim et al., in DNA Cloning: Mammalian Systems, D. Glover, Ed. (Oxford Univ. Press, Oxford England) (1995); Geller et al., Proc Natl. Acad. Sci. U.S.A. 90:7603 (1993); Geller et al., Proc Natl. Acad. Sci USA 87:1149 (1990)), Ad Vectors (LaSalle et al., Science 259:988 (1993); Davidson et al., Nat. Genet. 3:219 (1993); Yang et al., J. Virol. 69:2004 (1995)), and AAV Vectors (Kaplitt et al., Nat. Genet. 8:148 (1994)).

If desired, the polynucleotides described here may also be used with a microdelivery vehicle such as cationic liposomes, adenoviral vectors, and exosomes. For a review of the procedures for liposome preparation, targeting and delivery of contents, see Mannino and Gould-Fogerite, BioTechniques 6:682 (1988). See also, Feigner and Holm, Bethesda Res. Lab. Focus 11:21 (1989) and Maurer, Bethesda Res. Lab. Focus 11:25 (1989). In some embodiments, exosomes may be used for delivery of a nucleic acid encoding a CRISPR endonuclease and/or guide RNA to a target cell, e.g. a cancer cell. Exosomes are nanosized vesicles secreted by a variety of cells and are comprised of cellular membranes. Exosomes can attach to target cells by a range of surface adhesion proteins and vector ligands (tetraspanins, integrins, CD11 b and CD18 receptors), and deliver their payload to target cells. Several studies indicate that exosomes have a specific cell tropism, according to their characteristics and origin, which can be used to target them to disease tissues and/or organs. See Batrakova et al., J. Control. Release 219:396-405 (2015). For example, cancer-derived exosomes function as natural carriers that can efficiently deliver CRISPR/Cas9 plasmids to cancer cells. See Kim et al., J. Control. Release 266:8-16 (2017). In some embodiments, RNPs (discussed further below) are loaded into exosomes for delivery. See, e.g., Wan et al., Sci. Adv. 8:eabp9435 (2022).

Replication-defective recombinant adenoviral vectors, can be produced in accordance with known techniques. See Quantin et al., Proc. Natl. Acad. Sci. USA 89:2581-84 (1992); Stratford-Perricadet et al., J. Clin. Invest. 90:626-30 (1992); and Rosenfeld et al., Cell 68143-55 (1992).

Another delivery method is to use single stranded DNA producing vectors which can produce the expressed products intracellularly. See, e.g., Chen et al., BioTechnique, 34:167-71 (2003).

Introduction of CRISPR/Cas systems can be accomplished by lipid nanoparticle (LNP)-mediated delivery. For example, LNP-mediated delivery can be used to deliver a combination of Cas mRNA and guide RNA or a combination of Cas protein and guide RNA. Delivery through such methods results in transient Cas expression, and the biodegradable lipids improve clearance, improve tolerability, and decrease immunogenicity. Lipid formulations can protect biological molecules from degradation while improving their cellular uptake.

LNPs are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840, herein incorporated by reference in its entirety for all purposes. An exemplary lipid nanoparticle can comprise a cationic lipid and one or more other components. In one embodiment, the other component can comprise a helper lipid such as cholesterol. In another embodiment, the other components can comprise a helper lipid such as cholesterol and a neutral lipid such as distearoylphosphatidylcholine (DSPC).

An LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al., Cell Rep. 22:1-9 (2018) and WO 2017/173054, each of which is herein incorporated by reference in its entirety for all purposes. In certain LNPs, the cargo can include a guide RNA or a nucleic acid encoding a guide RNA. In certain LNPs, the cargo can include an exogenous donor nucleic acid. In certain LNPs, the cargo can include a guide RNA or a nucleic acid encoding a guide RNA and a Cas protein or a nucleic acid encoding a Cas protein. In certain LNPs, the cargo can include a guide RNA or a nucleic acid encoding a guide RNA, a Cas protein or a nucleic acid encoding a Cas protein, and an exogenous donor nucleic acid.

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy-)carbonyl)oxy)methyl)propyl octadeca-9,12-dienoate, also called 3-((4,4-bis(octyloxy)butanoyl)oxy)-2-((((3-(diethylamino)propoxy)carbonyl-)oxy)methyl)propyl (9Z,12Z)-octadeca-9,12-dienoate. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1-,3-diyl(9Z,9Z′,12Z,12Z′)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as Dlin-MC3-DMA (MC3))).

Cationic lipid can be present in embodiments of the composition and lipid particles can comprise an amount from about 30 to about 60 mole percent (“mol %”, or the percentage of the total moles that is of a particular component), from about 30 mol % to about 55 mol %, from about 30 mol % to about 50 mol %, from about 30 mol % to about 45 mol %, from about 30 mol % to about 40 mol %, from about 30 mol % to about 35 mol %, from about 35 mol % to about 60 mol %, from about 40 mol % to about 60 mol %, from about 45 mol % to about 60 mol %, from about 50 mol % to about 60 mol %, from about 55 mol % to about 60 mol %, from about 35 mol % to about 55 mol %, from about 40 mol % to about 50 mol %. In in some embodiments, the cationic lipid is present in about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral (also termed structural) lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and combinations thereof. For example, the neutral phospholipid may be selected from the group consisting of distearoylphosphatidylcholine (DSPC) and dimyristoyl phosphatidyl ethanolamine (DMPE).

In certain embodiments, a neutral lipid is present in the lipid particle in an amount from about 20 mol % to about 40 mol %, from about 20 mol % to about 35 mol %, from about 20 mol % to about 30 mol %, from about 20 mol % to about 25 mol %, from about 25 mol % to about 40 mol %, from about 30 mol % to about 40 mol %, from about 30 mol % to about 40 mol %, from about 35 mol % to about 40 mol %, from about 25 mol % to about 35 mol %. In in some embodiments, the cationic lipid is present in about 20, 21, 22, 23, 24, 25, 26, 27, 28, 29, 30, 31, 32, 33, 34, 35, 36, 37, 38, 39, or 40 mol %.

In some embodiments, the lipids can be any of the lipids disclosed in US20210251898, US20210220449, US20210128488, US20210122703, US20210122702, US20210113483, US20210107861, US20210095309, US20210087135, US20190292566 each incorporated herein by reference in its entirety.

Commercially available LNPs include, e.g., Lipofectamine™ CRISPRMAX™ Cas9 Transfection Reagent (available from ThermoFisher Scientific, Waltham, MA), Pro-DeliverIN™ CRISPR Transfection Reagent (available from Oz Biosciences, San Diego, CA), and NanoAssemblr® LNPs (available from Precision NanoSystems, Vancouver, BC).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety. The hydrophilic head group of stealth lipid can comprise, for example, a polymer moiety selected from polymers based on poly(ethylene glycol), poly(oxazoline), poly(vinyl alcohol), poly(glycerol), poly(N-vinylpyrrolidone), polyaminoacids, and poly N-(2-hydroxypropyl)methacrylamide.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

In some embodiments, the stealth lipid may be PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-w-methyl-poly(ethylene glycol), PEG-DMB (3,4-ditetradecoxylbenzyl-ω-methyl-poly(ethylene glycol)ether), 1,2-dimyristoyl-sn-glycero-3-phosphoethanolamine-N-[methoxy(polyethylene glycol)-2000](PEG2k-DMG), 1,2-distearoyl-sn-glycero-3-phosphoethanolamine-N-[methoxy(polyethylene glycol)-2000](PEG2k-DSPE), 1,2-distearoyl-sn-glycerol, methoxypoly ethylene glycol (PEG2k-DSG), poly(ethylene glycol)-2000-dimethacrylate (PEG2k-DMA), or 1,2-distearyloxypropyl-3-amine-N-[methoxy(polyethylene glycol)-2000](PEG2k-DSA).

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5.

In some LNPs, the cargo can comprise Cas mRNA and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, 25:1, 10:1, 5:1, 3:1, 1:1, 1:3, 1:5, 1:10, or 1:25.

In some LNPs, the cargo can comprise exogenous donor nucleic acid and gRNA. The exogenous donor nucleic acid and gRNAs can be in different ratios. For example, the LNP formulation can include a ratio of exogenous donor nucleic acid to gRNA nucleic acid ranging from about 25:1 to about 1:25, ranging from about 10:1 to about 1:10, ranging from about 5:1 to about 1:5, or about 1:1. Alternatively, the LNP formulation can include a ratio of exogenous donor nucleic acid to gRNA nucleic acid from about 1:1 to about 1:5, about 5:1 to about 1:1, about 10:1, or about 1:10. Alternatively, the LNP formulation can include a ratio of exogenous donor nucleic acid to gRNA nucleic acid of about 1:10, 25:1, 10:1, 5:1, 3:1, 2.5:1, 1:1, 1:2.5, 1:3, 1:5, 1:10, or 1:25.

In some embodiments, one or more CRISPR endonucleases and one or more guide RNAs may be provided in combination in the form of ribonucleoprotein particles (RNPs). An RNP complex can be introduced into a subject by means of, e.g., injection, electroporation, nanoparticles (including, e.g., lipid nanoparticles), vesicles, and/or with the assistance of cell-penetrating peptides. See, e.g., Lin et al., ELife 3:e04766 (2014); Sansbury et al., CRISPR J. 2:121-32 (2019); US2019/0359973).

LNP particles can have a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm. LNPs may be made from cationic, anionic, or neutral lipids. Neutral lipids, such as the fusogenic phospholipid DOPE or the membrane component cholesterol, may be included in LNPs as “helper lipids” to enhance transfection activity and nanoparticle stability. LNPs may also be comprised of hydrophobic lipids, hydrophilic lipids, or both hydrophobic and hydrophilic lipids.

In certain embodiments, the cationic lipid N-[1-(2,3-dioleyloxy)propyl]-N,N,N-trimethylammonium chloride (DOTMA) can be used. DOTMA can be formulated alone or combined with the neutral lipid, dioleoylphosphatidyl-ethanolamine (DOPE) or other cationic or non-cationic lipids into a liposomal transfer vehicle or a lipid nanoparticle, and such liposomes can be used to enhance the delivery of nucleic acids into target cells. Other suitable cationic lipids include, but are not limited to, 5-carboxyspermylglycinedioctadecylamide, 2,3-dioleyloxy-N-[2(spermine-carboxamido)ethyl]-N,N-dimethyl-1-propanaminium, 1,2-Dioleoyl-3-Dimethylammonium-Propane, 1,2-Dioleoyl-3-Trimethylammonium-Propane. Contemplated cationic lipids also include 1,2-distearyloxy-N,N-dimethyl-3-aminopropane, 1,2-dioleyloxy-N,N-dimethyl-3-aminopropane, 1,2-dilinoleyloxy-N,N-dimethyl-3-aminopropane, 1,2-dilinolenyloxy-N,N-dimethyl-3-aminopropane, N-dioleyl-N,N-dimethylammonium chloride, N,N-distearyl-N,N-dimethylammonium bromide, N-(1,2-dimyristyloxyprop-3-yl)-N,N-dimethyl-N-hydroxyethyl ammonium bromide, 3-dimethylamino-2-(cholest-5-en-3-beta-oxybutan-4-oxy)-1-(cis,ci-s-9,12-octadecadienoxy)propane, 2-[5′-(cholest-5-en-3-beta-oxy)-3′-oxapentoxy)-3-dimethyl-1-(cis,cis-9′,12′-octadecadienoxy)propane, N,N-dimethyl-3,4-dioleyloxybenzylamine, 1,2-N,N′-dioleylcarbamyl-3-dimethylaminopropane, 2,3-Dilinoleoyloxy-N,N-dimethylpropylamine, 1,2-N,N′-Dilinoleylcarbamyl-3-dimethylaminopropane, 1,2-Dilinoleoylcarbamyl-3-dimethylaminopropane, 2,2-dilinoleyl-4-dimethylaminomethyl-[1,3]-dioxolane, 2,2-dilinoleyl-4-dimethylaminoethyl-[1,3]-dioxolane, and 2-(2,2-di((9Z,12Z)-octadeca-9,12-dien-1-yl)-1,3-dioxolan-4-yl)-N,N-dimethylethanamine (DLin-KC2-DMA)), or mixtures thereof.

In some embodiments, non-cationic lipids can be used. As used herein, the phrase “non-cationic lipid” refers to any neutral, zwitterionic, or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), DOPE, palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone or can be used in combination with other excipients, for example, cationic lipids.

### Virus-Like Particles

A recombinant expression vector sequence can be packaged into a virus or virus-like particle (also referred to herein as a “particle” or “virion”) for subsequent infection and transformation of a cell, ex vivo, in vitro, or in vivo. Such particles or virions will typically include proteins that encapsidate or package the vector genome. Suitable expression vectors may include viral expression vectors based on vaccinia virus; poliovirus; adenovirus; a retroviral vector (e.g., Murine Leukemia Virus), spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus; and the like. In some embodiments, a recombinant expression vector of the present disclosure is a recombinant adeno-associated virus (AAV) vector. In some embodiments, a recombinant expression vector of the present disclosure is a recombinant lentivirus vector. In some embodiments, a recombinant expression vector of the present disclosure is a recombinant retroviral vector.

In other embodiments, suitable vectors may include virus-like particles (VLP). Virus-like particles (VLPs) are particles that closely resemble viruses, but do not contain viral genetic material and are therefore non-infectious. In some embodiments, VLPs comprise a polynucleotide encoding a transgene of interest, for example any Cas protein and/or a gRNA embodiments, and, optionally, donor template polynucleotides described herein, packaged with one or more viral structural proteins.

In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 17: 767-77 (2009)).

### NRF2

NRF2 (Nuclear Factor Erythroid 2-Related Factor-Like 2) is a master regulator of 100-200 target genes involved in cellular responses to oxidative/electrophilic stress. While transient activation in response to stress is beneficial, constitutive NRF2 activation in cancer cells has deleterious effects on the host, by amplifying the antioxidant and detoxification capability of cancer cells and driving metabolic reprogramming to establish cellular metabolic processes advantageous for cell proliferation in cooperation with other oncogenic pathways. This in turn confers therapeutic resistance and activates aggressive tumorigenic activity on cancer cells. Mechanism of NRF2 activation include somatic mutation and copy number variation in NFE2L2 gene (gain of function or amplification) and KEAP1 (loss of function or deletion). KEAP (Kelch-like ECH associated protein1) is a E3 ubiquitin ligase substrate adaptor, which targets NRF2 for proteasomal degradation under basal condition thus serving as a negative regulator. Mutations disrupt KEAP1 binding and lead to constitutive expression of NRF2 in cancer cells. These mutations are frequently found in solid tumors especially in head and neck (25%), lung (11%), colon (8%), liver (9%) breast (2%). Other than this, mutation of EGFR, Kras, Braf, Myc, and the Bcr-Abl fusion can activate NRF2, resulting in enhancement of ROS detoxification and induction of chemoresistance in cancer cells. NRF2 stabilization leads to its translocation into the nucleus where it binds to antioxidant response element (ARE) in promoter regions of various genes that regulate the cellular response to oxidative and xenobiotic stress. NRF2 target genes include antioxidant genes and phase II enzymes such as heme oxygenase-1 (HO-1), NAD(P)H: quinone oxidoreductase 1 (NQO1), glutathione S-transferase (GST), and glutathione peroxidase. Hence, NRF2 qualifies as an attractive molecular target for a number of reasons: 1) it is a far upstream component of the pathway that leads to resistance to many forms of cancer therapy; 2) it is a master regulator, transcription factor, so disabling the gene that encodes this factor has a large downstream of fact on multiple genes that block drug action; 3) it is heavily up regulated during the tumors. Disruption of NRF2 increases the efficacy of chemotherapy in NRF2 chemoresistance tumor by loss of NRF2 protein expression.

NRF2 is considered the master regulator of 100-200 target genes involved in cellular responses to oxidative/electrophilic stress. Targets include glutathione (GSH) mediators, antioxidants and genes controlling efflux pumps. (Hayden et al., Urol. Oncol. Semin. Orig. Investig. 32:806-14 (2014)). NRF2 is also known to regulate expression of genes involved in protein degradation and detoxification and is negatively regulated by KEAP1, a substrate adapter for the Cul3-dependent E3 ubiquitin ligase complex. The upregulation of NRF2 expression leads to an enhanced resistance of cancer cells to chemotherapeutic drugs, which by their very action induce an unfavorable environment for cell proliferation. Indeed, Hayden et al. (2014) demonstrated that increased NRF2 expression leads to the resistance of cancer cells to chemotherapeutic drugs including cisplatin. Singh et al., Antioxid. Redox Signal. 13:1627-37 (2010), also showed that constitutive expression of NRF2 leads to radioresistance, and inhibition of NRF2 causes increased endogenous reactive oxygen species (ROS) levels as well as decreased survival. Torrente et al., Oncogene 36:6204-12 (2017), identified crosstalk between NRF2 and the homeodomain interacting protein kinase two, HIPK2, demonstrating that HIPK2 exhibits a cytoprotective effect through NRF2.

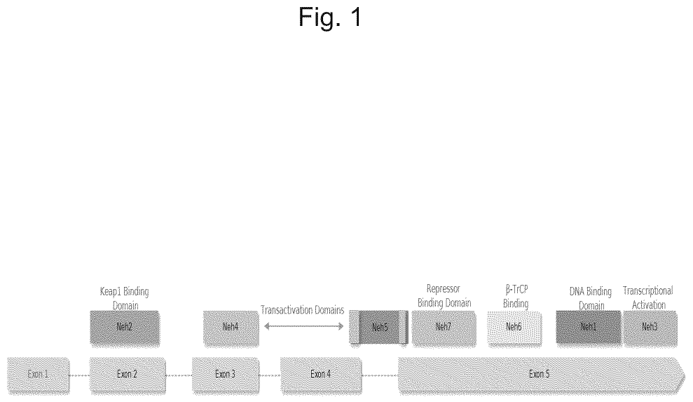

Nrf2 contains seven conserved regions named Nrf2-ECH homology (Neh) domain (FIG. 1). The Neh1 domain contains a CNC homology region and a basic leucine zipper domain, which allows nrf2 to form a heterodimer complex with small Maf and then bind to the ARE region in target genes. The Neh2 domain negatively regulates Nrf2 through binding of its DLG and ETGE motifs to Keap1. The Neh3 domain allows Nrf2 to interact with a chromo-ATPase/helicase DNA binding protein CHD6, which functions as an Nrf2 transcriptional activator. The Neh4 and Neh5 domains adjacent to Neh2 cooperatively bind to cAMP responsive element binding protein binding protein (CBP) and Brahma-related gene 1 (BRG1). The Neh6 domain contains two phosphorylation-dependent destruction motifs (DSGIS and DSAPGS) recognized by beta-transducin repeats-containing proteins (β-TrCP) of Skp-Cullin1-F-box protein E3 ubiquitin ligase complex. Neh7 domain is a region through which retinoic X receptor alpha binds to and subsequently suppresses the transcriptional activity of Nrf2 (Ngo et al., Nrf2, a target for precision oncology in cancer prognosis and treatment, J. Cancer Prev., 28:131-42 (2023); Zhang et al., Nrf2 Neh5 domain is differentially utilized in the transactivation of cytoprotective genes, Biocherr. J, 404:459-66 (2007)).

The N-terminal region of Nrf2 contains two transactivation domains, Neh4 and Neh5, and both domains are indispensable for the maximum transactivation activity of Nrf2. Neh4 and Neh5 cooperatively bind to CBP and synergistically activate reporter gene expression via the ARE, and CBP is required for the activity of Nrf2. CBP and its close homologue, p300, have been shown to play essential roles as co-activators of many classes of sequence specific transcription factors in a variety of signal-modulated cellular events (Katoh et al., Two domains of Nrf2 cooperatively bind CBP, a CREB binding protein, and synergistically activate transcription. Genes Cells, 6:857-68 (2001)). Applicant's here target the exon 3 and exon 4 domains with CRISPR/Cas systems to reduce, and in some embodiments eliminate, Nrf2 transcriptional activity. In some embodiments, Applicant targets PAM sites within, e.g., exon 3 or exon 4 of the NRF2 gene to knockout portions or all of Neh4 and/or Neh5 domains.

In some embodiments, the human NRF2 gene has the sequence set forth in RefSeq NM_006164.5 (SEQ ID NO:1 below), which encodes the human nrf2 protein having the amino acid sequence set forth in RefSeq NP_006155.2 (SEQ ID NO:2 below).

SEQ ID NO: 1
gattaccgag tgccggggag cccggaggag ccgccgacgc agccgccacc gccgccgccg
ccgccaccag agccgccctg tccgcgccgc gcctcggcag ccggaacagg gccgccgtcg gggagcccca
acacacggtc cacagctcat catgatggac ttggagctgc cgccgccggg actcccgtcc cagcaggaca
tggatttgat tgacatactt tggaggcaag atatagatct tggagtaagt cgagaagtat ttgacttcag
tcagcgacgg aaagagtatg agctggaaaa acagaaaaaa cttgaaaagg aaagacaaga acaactccaa
aaggagcaag agaaagcctt tttcgctcag ttacaactag atgaagagac aggtgaattt ctcccaattc
agccagccca gcacatccag tcagaaacca gtggatctgc caactactcc caggttgccc acattcccaa
atcagatgct ttgtactttg atgactgcat gcagcttttg gcgcagacat tcccgtttgt agatgacaat
gaggtttctt cggctacgtt tcagtcactt gttcctgata ttcccggtca catcgagagc ccagtcttca
ttgctactaa tcaggctcag tcacctgaaa cttctgttgc tcaggtagcc cctgttgatt tagacggtat
gcaacaggac attgagcaag tttgggagga gctattatcc attcctgagt tacagtgtct taatattgaa
aatgacaagc tggttgagac taccatggtt ccaagtccag aagccaaact gacagaagtt gacaattatc
atttttactc atctataccc tcaatggaaa aagaagtagg taactgtagt ccacattttc ttaatgcttt
tgaggattcc ttcagcagca tcctctccac agaagacccc aaccagttga cagtgaactc attaaattca
gatgccacag tcaacacaga ttttggtgat gaattttatt ctgctttcat agctgagccc agtatcagca
acagcatgcc ctcacctgct actttaagcc attcactctc tgaacttcta aatgggccca ttgatgtttc
tgatctatca ctttgcaaag ctttcaacca aaaccaccct gaaagcacag cagaattcaa tgattctgac
tccggcattt cactaaacac aagtcccagt gtggcatcac cagaacactc agtggaatct tccagctatg
gagacacact acttggcctc agtgattctg aagtggaaga gctagatagt gcccctggaa gtgtcaaaca
gaatggtcct aaaacaccag tacattcttc tggggatatg gtacaaccct tgtcaccatc tcaggggcag
agcactcacg tgcatgatgc ccaatgtgag aacacaccag agaaagaatt gcctgtaagt cctggtcatc
ggaaaacccc attcacaaaa gacaaacatt caagccgctt ggaggctcat ctcacaagag atgaacttag
ggcaaaagct ctccatatcc cattccctgt agaaaaaatc attaacctcc ctgttgttga cttcaacgaa
atgatgtcca aagagcagtt caatgaagct caacttgcat taattcggga tatacgtagg aggggtaaga
ataaagtggc tgctcagaat tgcagaaaaa gaaaactgga aaatatagta gaactagagc aagatttaga
tcatttgaaa gatgaaaaag aaaaattgct caaagaaaaa ggagaaaatg acaaaagcct tcacctactg
aaaaaacaac tcagcacctt atatctcgaa gttttcagca tgctacgtga tgaagatgga aaaccttatt
ctcctagtga atactccctg cagcaaacaa gagatggcaa tgttttcctt gttcccaaaa gtaagaagcc
agatgttaag aaaaactaga tttaggagga tttgaccttt tctgagcta gtttttttgta ctattatact
aaaagctcct actgtgatgt gaaatgctca tactttataa gtaattctat gcaaaatcat agccaaaact
agtatagaaa ataatacgaa actttaaaaa gcattggagt gtcagtatgt tgaatcagta gtttcacttt
aactgtaaac aatttcttag gacaccattt gggctagttt ctgtgtaagt gtaaatacta caaaaactta
tttatactgt tcttatgtca tttgttatat tcatagattt atatgatgat atgacatctg gctaaaaaga
aattattgca aaactaacca ctatgtactt ttttataaat actgtatgga caaaaaatgg cattttttat
attaaattgt ttagctctgg caaaaaaaaa aaattttaag agctggtact aataaaggat tattatgact gttaaa
SEQ ID NO: 2
mmdlelpppg lpsqqdmdli dilwrqdidl gvsrevfdfs qrrkeyelek qkklekerqe qlqkeqekaf
faqlqldeet geflpiqpaq hiqsetsgsa nysqvahipk sdalyfddcm qllaqtfpfv ddnevssatf
qslvpdipgh iespvfiatn qaqspetsva qvapvdldgm qqdieqvwee Ilsipelqcl niendklvet
tmvpspeakl tevdnyhfys sipsmekevg ncsphflnaf edsfssilst edpnqltvns Insdatvntd
fgdefysafi aepsisnsmp spatlshsls ellngpidvs dlslckafnq nhpestaefn dsdsgisInt
spsvaspehs vesssygdtl Iglsdsevee Idsapgsvkq ngpktpvhss gdmvqplsps qgqsthvhda
qcentpekel pvspghrktp ftkdkhssrl eahltrdelr akalhipfpv ekiinlpvvd fnemmskeqf
neaqlalird irrrgknkva aqncrkrkle niveleqdld hlkdekekll kekgendksl hllkkqlstl
ylevfsmlrd edgkpyspse yslqqtrdgn vflvpkskkp dvkkn

In other embodiments, NRF2 has a sequence described in Ensembl ENSG00000116044, including any of the 65 transcripts described therein.

In some embodiments, an NRF2 gene or protein shares a percent sequence identity with the SEQ ID NO:1 and 2, respectively (or any of the transcript variants described in Ensembl ENSG00000116044). “Percent sequence identity” refers to the degree of sequence identity between any given reference sequence, e.g., SEQ ID NO: 1 or 2, and a variant NRF2 gene or nrf2 protein, or, e.g., any of the variants listed in the preceding paragraph and further variant NRF2 gene or nrf2 protein thereof. A candidate sequence typically has a length that is from 80% to 200% of the length of the reference sequence, e.g., 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) can be aligned to one or more candidate sequences using, e.g., ClustalW, Clustal 0, or BLAST, which allows alignments of nucleic acid or polypeptide sequences to be carried out across their entire length (Chenna et al., Nucleic Acids Res. 31:3497-3500 (2003)).

Typically, when an alignment is prepared based upon an amino acid sequence, the alignment contains insertions and deletions which are so identified with respect to a reference sequence and the numbering of the amino acid residues is based upon a reference scale provided for the alignment. However, any given reference sequence may have fewer amino acid residues than the reference scale. Herein, when discussing the parental sequence, the term “the same position” or the “corresponding position” refers to the amino acid located at the same residue number in each of the sequences, with respect to the reference scale for the aligned sequences. However, when taken out of the alignment, each of the proteins may have these amino acids located at different residue numbers.

Variants of a polypeptide (e.g., of nrf2) may also refer to a polypeptide comprising a referenced amino acid sequence except for one or more (e.g., 1, 2, 3, 4, 5, 6, 7, 8, 9 or 10) mutations such as, for example, missense mutations (e.g., conservative substitutions), nonsense mutations, deletions, or insertions.

### Pharmaceutical Compositions

Any of the pharmaceutical compositions disclosed herein can be formulated for use in the preparation of a medicament, and particular uses are indicated below in the context of treatment, e.g., the treatment of a subject having cancer. When employed as pharmaceuticals, any of the nucleic acids and vectors can be administered in the form of pharmaceutical compositions. Administration may be pulmonary (e.g., by inhalation or insufflation of powders or aerosols, including by nebulizer; intratracheal, intranasal, epidermal and transdermal), topical (including ophthalmic and to mucous membranes including intranasal, vaginal and rectal delivery), ocular, oral or parenteral. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

In some embodiments, pharmaceutical compositions can contain, as the active ingredient, nucleic acids, vectors, and/or RNPs described herein in combination with one or more pharmaceutically acceptable carriers. The term “pharmaceutically acceptable” refers to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the pharmaceutical compositions disclosed herein, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences disclosed herein can be delivered to an appropriate cell of a subject, e.g. a cancer cell. This can be achieved by, for example, the use of a polymeric, biodegradable microparticle or microcapsule delivery vehicle, sized to optimize phagocytosis by phagocytic cells such as macrophages. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the isolated nucleic acid sequence comprising a sequence encoding a CRISPR-associated endonuclease and a guide RNA can be operatively linked to a promoter or enhancer-promoter combination. Promoters and enhancers are described above.

In some embodiments, the pharmaceutical compositions can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids, vectors, and RNPs may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in, e.g., Remington: The Science and Practice of Pharmacy (Adejare, ed., 23^{rd} Edition, Nov. 13, 2020) and in the USP/NF (United States Pharmacopeia and the National Formulary).

In some embodiments, the compositions can be formulated as a nanoparticle encapsulating a nucleic acid encoding a CRISPR-associated endonuclease and a guide RNA sequence complementary to an NRF2 gene (or, in some embodiments, a variant NRF2 gene), or vector comprising a nucleic acid encoding a CRISPR-associated endonuclease and a guide RNA sequence complementary to an NRF2 gene (or, in some embodiments, a variant NRF2 gene).

The concentration of anti-cancer therapies can vary widely, and will be selected primarily based on fluid volumes, viscosities, body weight and the like in accordance with the particular mode of administration selected and the biological system's needs. Generally, the amount of the anti-cancer therapy or therapies present in a pharmaceutical composition will be that which will produce a therapeutic effect. For example, in some embodiments, the weight per volume (w/v) or weight percent (wt %) concentration of an anti-cancer therapy or therapies in a pharmaceutical composition may be between about 0.001% to 100%, 0.001% to 90%, 0.001% to 80%, 0.001% to 70%, 0.001% to 60%, 0.001% to 50%, 0.001% to 40%, 0.001% to 30%, 0.001% to 20%, 0.001% to 10%, 0.001% to 1%, 0.01% to 100%, 0.01% to 90%, 0.01% to 80%, 0.01% to 70%, 0.01% to 60%, 0.01% to 50%, 0.01% to 40%, 0.01% to 30%, 0.01% to 20%, 0.01% to 10%, 0.01% to 1%, 0.1% to 100%, 0.1% to 90%, 0.1% to 80%, 0.1% to 70%, 0.1% to 60%, 0.1% to 50%, 0.1% to 40%, 0.1% to 30%, 0.1% to 20%, 0.1% to 10%, 0.1% to 1%, 1% to 100%, 1% to 90%, 1% to 80%, 1% to 70%, 1% to 60%, 1% to 50%, 1% to 40%, 1% to 30%, 1% to 20%, 1% to 10%, 1% to 5%, 1% to 4%, 1% to 3%, 1% to 2%, 0.1% to 0.9%, 0.1% to 0.8%, 0.1% to 0.7%, 0.1% to 0.6%, 0.1% to 0.5%, 0.1% to 0.4%, 0.1% to 0.3%, 0.1% to 0.2%, 0.2% to 1%, 0.3% to 1%, 0.4% to 1%, 0.5% to 1%, 0.6% to 1%, 0.7% to 1%, 0.8% to 1%, or 0.9% to 1%.

In other embodiments, the concentration of an anti-cancer therapy or therapies in a pharmaceutical composition may be about 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM. 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, or 1M. In some aspects, the concentration (molarity or wt %) of an anti-cancer therapy or therapies that produces a therapeutic effect in a subject (e.g., a human or other mammal) can be extrapolated from in vitro or in vivo data, from cell culture and/or animal experiments.

### Methods of Reducing Gene Expression or Activity in a Cell

In certain aspects, the disclosure relates to a method of reducing NRF2 expression or activity in a cell comprising introducing into the cell (a) one or more DNA sequence(s) encoding a guide RNA (gRNA) that is complementary to a target sequence in the NRF2 gene and (b) a nucleic acid sequence encoding a Clustered Regularly Interspaced Short Palindromic Repeat (CRISPR)-associated endonuclease, whereby the gRNA hybridizes to the NRF2 gene and the CRISPR-associated endonuclease cleaves the NRF2 gene.

In some embodiments, the gRNA is one or more of the gRNA set forth in SEQ ID NO: 3-74, as shown in Table 1 below.

TABLE 1
Description of Sequences

| SEQ | | |
|---|---|---|
| ID NO: | Description | Sequence |
| 3 | spCas9-1 | TTGTGTCATTCCCTTTTATC AGG |
| 4 | spCas9-2 | AATGTGGGCAACCTGATAAA AGG |
| 5 | gRNA 4 | AGCATCTGATTTGGGAATGT GGG |
| 6 | spCas9-4 | AAGCATCTGATTTGGGAATG TGG |
| 7 | spCas9-5 | AAGTACAAAGCATCTGATTT GGG |
| 8 | spCas9-6 | AAAGTACAAAGCATCTGATT TGG |
| 9 | gRNA 73 | TGATGACTGCATGCAGCTTT TGG |
| 10 | gRNA 5 | CCTCATTGTCATCTACAAAC GGG |
| 11 | spCas9-9 | ACCTCATTGTCATCTACAAA CGG |
| 12 | spCas9-10 | CCCGTTTGTAGATGACAATG AGG |
| 13 | gRNA 116 | TTGTAGATGACAATGAGGTG AGG |
| 14 | spCas9-12 | ATCAATGCCTTATCAATTTT AGG |
| 15 | spCas9-13 | TTATCAATTTTAGGTTTCTT CGG |
| 16 | gRNA 76 | TCACTTGTTCCTGATATTCC CGG |
| 17 | gRNA 1 | TCGATGTGACCGGGAATATC AGG |
| 18 | gRNA 77 | GACTGGGCTCTCGATGTGAC CGG |
| 19 | spCas9-17 | GATTAGTAGCAATGAAGACT GGG |
| 20 | spCas9-18 | TGATTAGTAGCAATGAAGAC TGG |
| 21 | spCas9-19 | AGTCTTCATTGCTACTAATC AGG |
| 22 | spCas9-20 | ACCTGAAACTTCTGTTGCTC AGG |
| 23 | spCas9-21 | ACCTGAGCAACAGAAGTTTC AGG |
| 24 | gRNA 83 | GTAGCCCCTGTTGATTTAGA CGG |
| 25 | spCas9-23 | CATACCGTCTAAATCAACAG GGG |
| 26 | spCas9-24 | GCATACCGTCTAAATCAACA GGG |
| 27 | spCas9-25 | TGCATACCGTCTAAATCAAC AGG |
| 28 | spCas9-26 | AACAGGACATTGAGCAAGTT TGG |
| 29 | spCas9-27 | ACAGGACATTGAGCAAGTTT GGG |
| 30 | spCas9-28 | GGACATTGAGCAAGTTTGGG AGG |
| 31 | spCas9-29 | AGTTACCTGTAACTCAGGAA TGG |
| 32 | spCas9-30 | ATTATCCATTCCTGAGTTAC AGG |
| 33 | spCas9-31 | ATTTTAGTTACCTGTAACTC AGG |
| 34 | gRNA 2 | TGATTTAGACGGTATGCAAC AGG |
| 35 | gRNA 94 | TGAGTTCACTGTCAACTGGT TGG |
| 36 | gRNA 107 | AGACAAACATTCAAGCCGCT TGG |
| 37 | saCas9-1 | CAAGTGACTGAAACGTAGCCG AAGAA |
| 38 | saCas9-2 | GGGAGGAGCTATTATCCATTC CTGAG |
| 39 | saCas9-3 | TTCCTGATATTCCCGGTCACA TCGAG |
| 40 | saCas9-4 | GAACAAGTGACTGAAACGTAG CCGAA |
| 41 | saCas9-5 | AAAGTACAAAGCATCTGATTT GGGAA |
| 42 | saCas9-6 | TTGCATACCGTCTAAATCAAC AGGGG |
| 43 | saCas9-7 | GACTGAGCCTGATTAGTAGCA ATGAA |
| 44 | saCas9-8 | AAGACTGGGCTCTCGATGTGA CCGGG |
| 45 | saCas9-9 | GGGAATATCAGGAACAAGTGA CTGAA |
| 46 | saCas9-10 | GACTGGGCTCTCGATGTGACC GGGAA |
| 47 | saCas9-11 | GTTGCATACCGTCTAAATCAA CAGGG |
| 48 | saCas9-12 | AGACTGGGCTCTCGATGTGAC CGGGA |
| 49 | saCas9-13 | ACCTCATTGTCATCTACAAAC GGGAA |
| 50 | saCas9-14 | TAGACGGTATGCAACAGGACA TTGAG |
| 51 | saCas9-15 | GTCTAAATCAACAGGGGCTAC CTGAG |
| 52 | saCas9-16 | CCTGATATTCCCGGTCACATC GAGAG |
| 53 | saCas9-17 | CAGGACATTGAGCAAGTTTGG GAGGA |
| 54 | saCas9-18 | AACAGGACATTGAGCAAGTTT GGGAG |
| 55 | saCas9-19 | AGCAACAGAAGTTTCAGGTGA CTGAG |
| 56 | saCas9-20 | CAACAGGGGCTACCTGAGCAA CAGAA |
| 57 | saCas9-21 | GTTGATTTAGACGGTATGCAA CAGGA |
| 58 | saCas9-22 | CTACTAATCAGGCTCAGTCAC CTGAA |
| 59 | saCas9-23 | AGGACATTGAGCAAGTTTGGG AGGAG |
| 60 | saCas9-24 | CCTGATTAGTAGCAATGAAGA CTGGG |
| 61 | saCas9-25 | CATTCCCGTTTGTAGATGACA ATGAG |
| 62 | saCas9-26 | CAAAGTACAAAGCATCTGATT TGGGA |
| 63 | saCas9-27 | CTCGATGTGACCGGGAATATC AGGAA |
| 64 | saCas9-28 | TCAAAGTACAAAGCATCTGAT TTGGG |
| 65 | saCas9-29 | AACCTCATTGTCATCTACAAA CGGGA |
| 66 | saCas9-30 | AAACCTCATTGTCATCTACAA ACGGG |
| 67 | Cas12a-1 | GGAGGAGCTATTATCCATTCCTG TTTG |
| 68 | Cas12a-2 | GACGGTATGCAACAGGACATTGA TTTA |
| 69 | Cas12a-3 | GCGCAGACATTCCCGTTTGTAGA TTTG |
| 70 | Cas12a-4 | AGGTGACTGAGCCTGATTAGTAG TTTC |
| 71 | Cas12a-5 | TAGATGACAATGAGGTTTCTTCG TTTG |
| 72 | Cas12a-6 | TTCGGCTACGTTTCAGTCACTTG TTTC |
| 73 | Cas12a-7 | AGTCACTTGTTCCTGATATTCCC TTTC |
| 74 | Cas12a-8 | TACTTTGATGACTGCATGCAGCT TTTG |

### Pam Sequence Underlined.

Reducing NRF2 expression in the cancer cell may comprise reducing expression of NRF2 mRNA in the cancer cell, reducing expression of the nrf2 protein in the cancer cell, or both. In some embodiments, expression of one or more allele(s) of the NRF2 gene is reduced. In some embodiments, introducing the one or more DNA sequence(s) encoding the gRNA and the nucleic acid sequence encoding a CRISPR-associated endonuclease into the cancer cell reduces NRF2 expression and/or activity in the cancer cell, but does not completely eliminate it. In other embodiments, NRF2 expression and/or activity in the cancer cell are completely eliminated.

The gRNA is complementary to a target sequence in exon 3 and/or exon 4 of the NRF2 gene. In some embodiments, the gRNA is encoded by a single DNA sequence. In other embodiments, the gRNA is encoded by two or more DNA sequences. For example, in some embodiments, the gRNA is encode by a first DNA sequence encoding a trans-activated small RNA (tracrRNA) and a second DNA sequence encoding a CRISPR RNA (crRNA). The tracrRNA and crRNA may hybridize within the cell to form the guide RNA. Accordingly, in some embodiments, the gRNA comprises a trans-activated small RNA (tracrRNA) and a CRISPR RNA (crRNA).

In some embodiments, the guide RNA is complementary to a variant NRF2 gene that is found only in cancer cells and not in the wild-type NRF2 gene in normal (i.e., non-cancerous) cells. In some embodiments, introducing the one or more DNA sequence(s) encoding the gRNA and the nucleic acid sequence encoding a CRISPR-associated endonuclease into the cancer cell reduces variant NRF2 expression and/or activity in the cancer cell, but does not completely eliminate it. In other embodiments, variant NRF2 expression and/or activity in the cancer cell are completely eliminated.

In some embodiments, CRISPR-associated endonucleases suitable for use in reducing expression of the NRF2 gene include, but are not limited to, a class 1 CRISPR-associated endonucleases such as, e.g., Cas7 and Cas5, along with, in some embodiments, SS (Cas11) and Cas8a1; Cas8b1; Cas8c; Cas8u2 and Cas6; Cas3″ and Cas10d; Cas SS (Cas11), Cas8e, and Cas6; Cas8f and Cas6f; Cas6f; Cas8-like (Csf1); SS (Cas11) and Cas8-like (Csf1); or SS (Cas11) and Cas10. Class 2 CRISPR-associated endonucleases include type I, type V, and type VI CRISPR-Cas systems, which have a single effector molecule. In some embodiments, CRISPR-associated endonucleases suitable for use in reducing expression of the NRF2 gene include, but are not limited to, class 2 CRISPR-associated endonucleases such as, e.g., Cas9, Cas12a (cpf1), Cas12b1 (c2c1), Cas12a2, Cas12b2, Cas12c (c2c3), Cas12d (CasY), Cas12e (CasX), Cas12f1 (Cas14a), Cas12f2 (Cas14b), Cas12f3 (Cas14c), Cas12g, Cas12h, Cas12i, Cas12j (Casϕ), Cas12k (c2c5), Cas13a (c2c2), Cas13b1 (c2c6), Cas13b2 (c2c6), Cas13bt, Cas13c (c2c7), Cas13ct, Cas13d, Cas13X, Cas13Y, c2c4, c2c8, c2c9, and/or c2c10.

Any cell containing an NRF2 gene may be suitable for use in the methods of reducing NRF2 expression or activity described herein. In some embodiments, the cell is a eukaryotic cell, e.g. a mammalian cell. In some embodiments, the cell is a human cell. In some embodiments, the NRF2 gene is a human NRF2 gene.

### Methods for Treatment of Cancer

In certain aspects, the disclosure relates to a method of treating cancer in a subject comprising administering to the subject a therapeutically effective amount of a pharmaceutical composition comprising a CRISPR-associated endonuclease and a guide RNA that is complementary to a target domain in exon 3 or 4 (in some embodiments, the Neh4 domain and/or the Neh5 domain) of an NRF2 gene in the subject. In certain aspects, the disclosure relates to a method of treating cancer in a subject comprising administering to the subject a therapeutically effective amount of a pharmaceutical composition comprising a CRISPR-associated endonuclease and a guide RNA that is complementary to a target domain in exon 3 or 4 of the NRF2 gene in a cancer cell in the subject.

In certain aspects, the disclosure relates to a method of treating cancer in a subject comprising administering to the subject a therapeutically effective amount of a pharmaceutical composition comprising: (a) a DNA sequence encoding a guide RNA that is complementary to a target domain from an NRF2 gene in the subject; and (b) a nucleic acid sequence encoding a CRISPR-associated endonuclease. In some embodiments, the guide RNA is complementary to a variant NRF2 gene that is found only in cancer cells and not in wild-type NRF2 genes in normal (i.e., non-cancerous) cells.

In certain embodiments, the cancer is treated only with the pharmaceutical composition comprising a CRISPR-associated endonuclease and a guide RNA that is complementary to a target domain from an NRF2 gene in the subject, or only with the pharmaceutical composition comprising: (a) a DNA sequence encoding a guide RNA that is complementary to a target domain from an NRF2 gene in the subject; and (b) a nucleic acid sequence encoding a CRISPR-associated endonuclease. In some embodiments, the guide RNA is complementary to a variant NRF2 gene that is found only in cancer cells and not in the wild-type NRF2 gene in normal (i.e., non-cancerous) cells. In certain embodiments, the cancer is treated with the pharmaceutical compositions as described herein and an additional agent, e.g. a chemotherapeutic agent. In certain embodiments, treatment with the chemotherapeutic agent is initiated at the same time as treatment with the pharmaceutical composition. In certain embodiments, the treatment with the chemotherapeutic agent is initiated after the treatment with the pharmaceutical composition is initiated. In certain embodiments, treatment with the chemotherapeutic agent is initiated at before the treatment with the pharmaceutical composition.

In certain embodiments, the pharmaceutical compositions of the present disclosure may be utilized for the treatment of cancer wherein the subject has failed at least one prior chemotherapeutic regimen. For example, in some embodiments, the cancer is resistant to one or more chemotherapeutic agents. Accordingly, the present disclosure provides methods of treating cancer in a subject, wherein the subject has failed at least one prior chemotherapeutic regimen for the cancer, comprising administering the pharmaceutical compositions as described herein to the subject in an amount sufficient to treat the cancer, thereby treating the cancer. The pharmaceutical compositions described herein may also be utilized for inhibiting tumor cell growth in a subject wherein the subject has failed at least one prior chemotherapeutic regimen. Accordingly, the present disclosure further provides methods of inhibiting tumor cell growth in a subject, e.g. wherein the subject has failed at least one prior chemotherapeutic regimen, comprising administering the pharmaceutical compositions described herein to the subject, such that tumor cell growth is inhibited. In certain embodiments, the subject is a mammal, e.g. a human.

For example, the pharmaceutical compositions described herein may be administered to a subject in an amount sufficient to reduce proliferation of cancer cells relative to cancer cells that are not treated with the pharmaceutical composition. The pharmaceutical composition may reduce cancer cell proliferation by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are not treated with the pharmaceutical composition.

In some embodiments, the pharmaceutical composition is administered in an amount sufficient to reduce tumor growth relative to a tumor that is not treated with the pharmaceutical composition. The pharmaceutical composition may reduce tumor growth by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are not treated with the pharmaceutical composition. In a particular embodiment, administration of the pharmaceutical composition to the subject completely inhibits tumor growth.

In one embodiment, administration of a pharmaceutical composition as described herein, achieves at least stable disease, reduces tumor size, inhibits tumor growth and/or prolongs the survival time of a tumor-bearing subject as compared to an appropriate control. Accordingly, this disclosure also relates to a method of treating tumors in a human, including a subject, who has failed at least one prior chemotherapeutic regimen, by administering to such human or animal an effective amount of a pharmaceutical composition described herein. One skilled in the art would be able, by routine experimentation with the guidance provided herein, to determine what an effective amount of the pharmaceutical composition would be for the purpose of treating malignancies including in a subject who has failed at least one prior chemotherapeutic regimen. For example, a therapeutically active amount of the pharmaceutical composition may vary according to factors such as the disease stage (e.g., stage I versus stage IV), age, sex, medical complications, and weight of the subject, and the ability of the pharmaceutical composition to elicit a desired response in the subject. The dosage regimen may be adjusted to provide the optimum therapeutic response. For example, several divided doses may be administered daily, the dose may be administered by continuous infusion, or the dose may be proportionally reduced as indicated by the exigencies of the therapeutic situation.

In certain embodiments, the methods further include a treatment regimen which includes any one of or a combination of surgery, radiation, chemotherapy, e.g., hormone therapy, antibody therapy, therapy with growth factors, cytokines, immunotherapy, targeted therapy, and anti-angiogenic therapy.

### Combination Therapies

In certain embodiments, the pharmaceutical compositions described herein can be used in combination therapy with at least one additional anticancer agent, e.g., a chemotherapeutic agent or an immunotherapy agent. Small molecule chemotherapeutic agents generally belong to various classes including, for example: 1. Topoisomerase II inhibitors (cytotoxic antibiotics), such as the anthracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; 2. Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; 3. Alkylating agents, such as nitrogen mustards, ethyleneimine compounds, alkyl sulphonates and other compounds with an alkylating action such as nitrosoureas, dacarbazine, cyclophosphamide, ifosfamide and melphalan; 4. Antimetabolites (nucleoside inhibitors), for example, folates, e.g., folic acid, fiuropyrimidines, purine or pyrimidine analogues such as 5-fluorouracil, capecitabine, gemcitabine, methotrexate, and edatrexate; 5. Topoisomerase I inhibitors, such as topotecan, irinotecan, and 9-nitrocamptothecin, camptothecin derivatives, and retinoic acid; and 6. Platinum compounds/complexes, such as cisplatin, oxaliplatin, and carboplatin. Exemplary chemotherapeutic agents for use in the methods of disclosed herein include, but are not limited to, amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-II, I0-hydroxy-7-ethyl-camptothecin (SN38), capecitabine, ftorafur, 5′deoxyflurouridine, UFT, eniluracil, deoxycytidine, 5-azacytosine, 5-azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973 (and analogs thereof), JM-216 (and analogs thereof), epirubicin, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, acyclovir, valacyclovir, ganciclovir, amantadine, rimantadine, lamivudine, zidovudine, bevacizumab, trastuzumab, rituximab, Pentostatin, floxuridine, fludarabine, hydroxyurea, ifosfamide, idarubicin, mesna, irinotecan, mitoxantrone, topotecan, leuprolide, megestrol, melphalan, plicamycin, mitotane, pegaspargase, pipobroman, tamoxifen, teniposide, testolactone, thiotepa, uracil mustard, vinorelbine, chlorambucil, mTor, epidermal growth factor receptor (EGFR), and fibroblast growth factors (FGF) and combinations thereof which are readily apparent to one of skill in the art based on the appropriate standard of care for a particular tumor or cancer. In a particular embodiment, the chemotherapeutic agent is selected from the group consisting of cisplatin, vinorelbine, carboplatin, and combinations thereof (e.g., cisplatin and vinorelbine; cisplatin and carboplatin; vinorelbine and carboplatin; cisplatin, vinorelbine, and carboplatin).

In some embodiments, the methods of the disclosure comprise administration of a cancer immunotherapy. Cancer immunotherapies can be categorized as active, passive, or hybrid (active and passive). These approaches exploit the fact that cancer cells often have molecules on their surface that can be detected by the immune system; they are often proteins or other macromolecules (e.g. carbohydrates). Active immunotherapy directs the immune system to attack tumor cells by targeting tumor antigens. Passive immunotherapies enhance existing anti-tumor responses and include the use of monoclonal antibodies, lymphocytes and cytokines.

In some embodiments, the immunotherapy agent is an anti-CTLA-4 agent, an anti-PD-1 agent, an anti-PD-L1 agent, an anti-PD-L2 agent, a TNF-α cross-linking agent, a TRAIL cross-linking agent, an anti-TWEAK agent, an anti-TWEAKR agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAMi agent, an anti-TIM3 agent, an anti-TIGIT agent, an anti-CD96 agent, an anti-CD70 agent, an anti-LIGHT agent, an anti-DR4 agent, an anti-CR5 agent an anti-CD95 agent, an anti-TRAIL agent, an anti-BCMA agent, an anti-TACI agent, an anti-RANKL agent, or an anti-BAFFR agent.

In some embodiments, the pharmaceutical composition is administered in an amount sufficient to reduce tumor growth relative to a tumor that is treated with the at least one chemotherapeutic agent but is not treated with the pharmaceutical composition. The pharmaceutical composition may reduce tumor growth by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are treated with the at least one chemotherapeutic agent but are not treated with the pharmaceutical composition.

### EXAMPLES

### Example 1: Gene Knockout of NRF2 Increases Chemosensitivity of Human Head and Neck (FADU) Cancer Cell

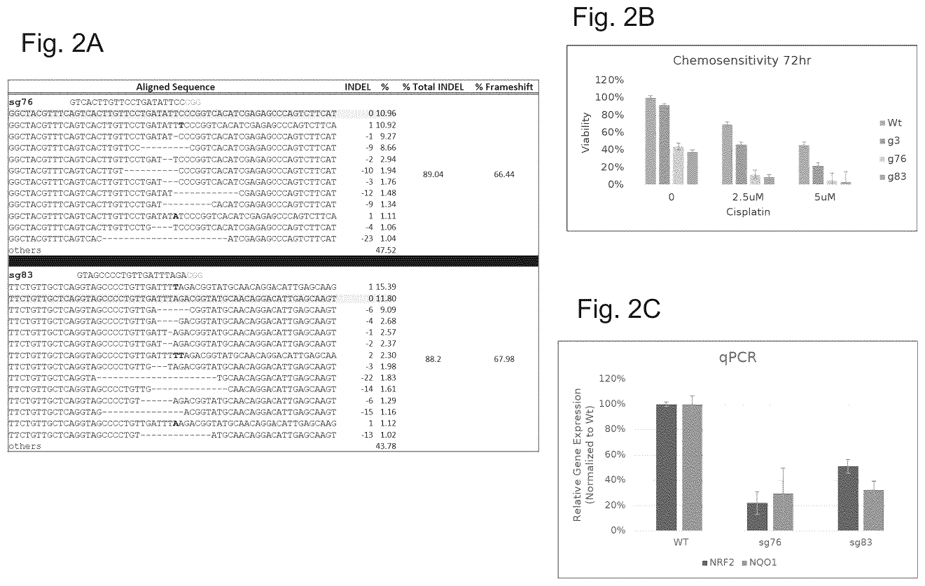

Disruption of the NRF2 gene through the CRISPR mediated knockout decreased cell viability in combination with chemotherapy. CRISPR gRNAs were designed across the NRF2 gene to impact functional domains of the protein (FIG. 1). FIG. 2 compares targeting exon 4 by two sgRNAs (sg76 (SEQ ID NO 16) and sg83 (SEQ ID NO 24)) and chemosensitivity because of targeting in the head and neck cancer, FaDu cell line. The DNA sequence data (FIG. 2A) indicates that each of the CRISPR molecules is efficient in knocking out NRF2 at exon 4, with enhanced sensitivity to cisplatin (FIG. 2B). Sensitivity to cisplatin is increased with only 36% viable cells at a 5 μM concentration compared to 55% for wildtype cells. The gene knockout of NRF2 alone is enough to decrease cell proliferation and viability as seen in the 0 μM panel. To confirm the loss of NRF2 activity, transcriptional activity of NRF2 was assessed by qPCR using a downstream target gene, NQO1, highly implicated and activated by NRF2 (FIG. 2C). As compared to the control, wildtype unedited cells, NQO1 expression is significantly decreased in both gRNA-treated cell populations, even without any cisplatin exposure.

### Example 2: Gene Knockout of NRF2 Increases Chemosensitivity of Human Esophageal (KYSE-410) Cancer Cell

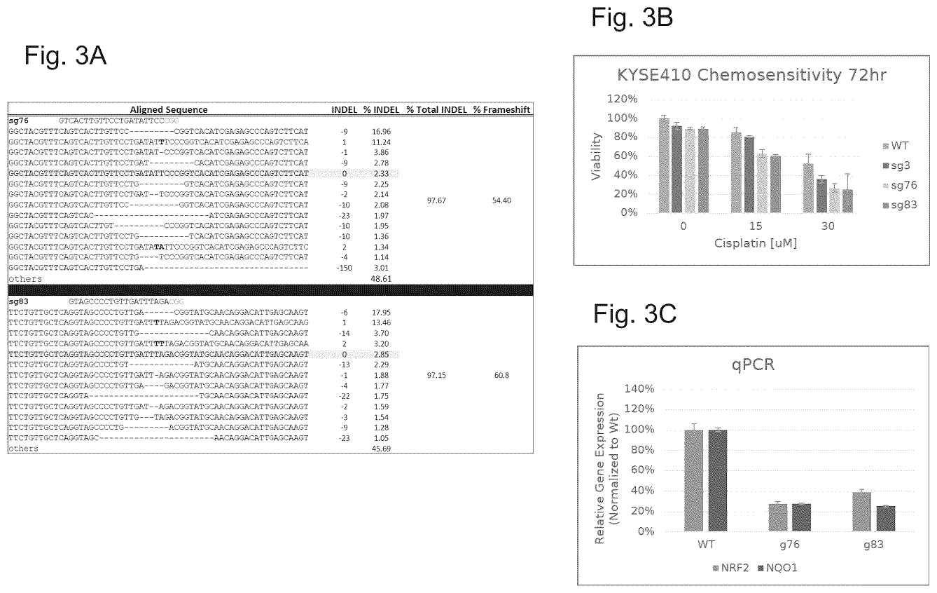

FIG. 3 compares targeting exon 4 by two sgRNAs (sg76 (SEQ ID NO 16) and sg83 (SEQ ID NO 24)) and chemosensitivity because of targeting in the esophageal cancer KYSE-410 cell line. The DNA sequence data (FIG. 3A) indicates that each of the CRISPR molecules is efficient in knocking out NRF2 at exon 4, with enhanced sensitivity to cisplatin (FIG. 3B). Sensitivity to cisplatin is increased with only 10% viable cells at a 30 μM concentration. The gene knockout of NRF2 alone is enough to decrease cell proliferation and viability as seen in the 0 μM panel. To confirm the loss of NRF2 activity, transcriptional activity of NRF2 was assessed by qPCR using a downstream target gene, NQO1, highly implicated and activated by NRF2 (FIG. 3C). As compared to the control, wildtype unedited cells, NQO1 expression is significantly decreased in both gRNA-treated cell populations, even without any cisplatin exposure.

### Example 3: Gene Knockout of NRF2 Increases Chemosensitivity of Human Lung Adenocarcinoma (A549) Cells

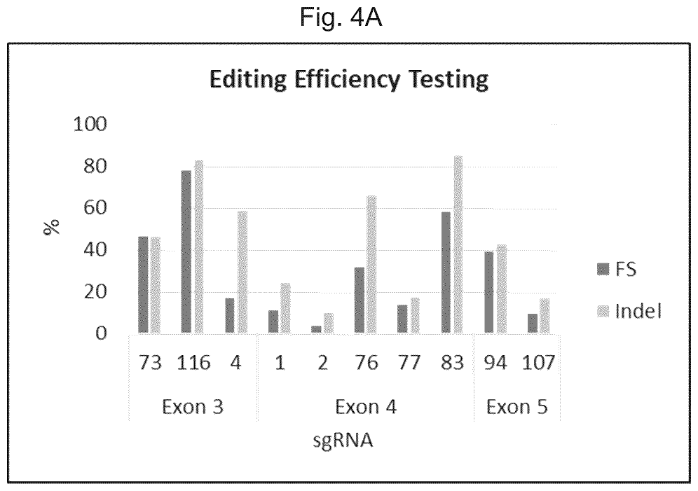

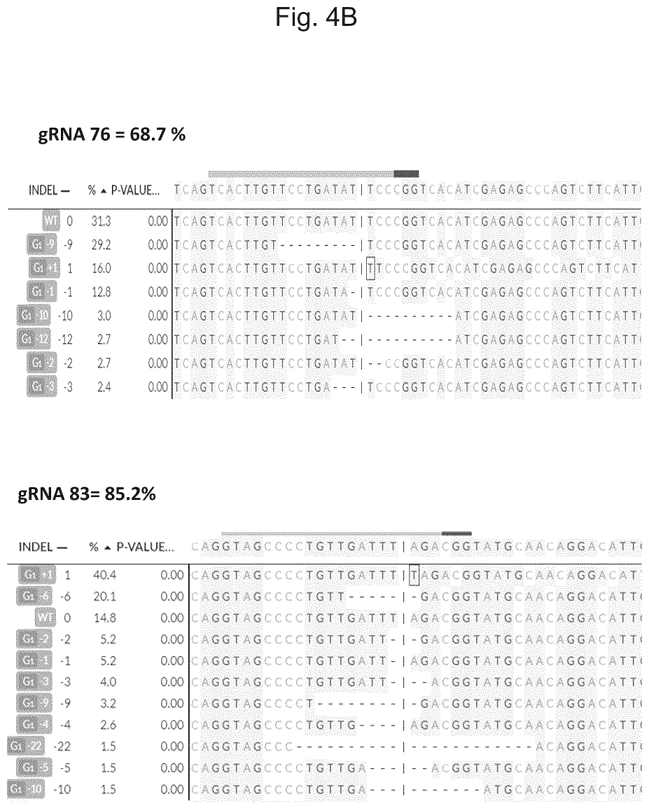

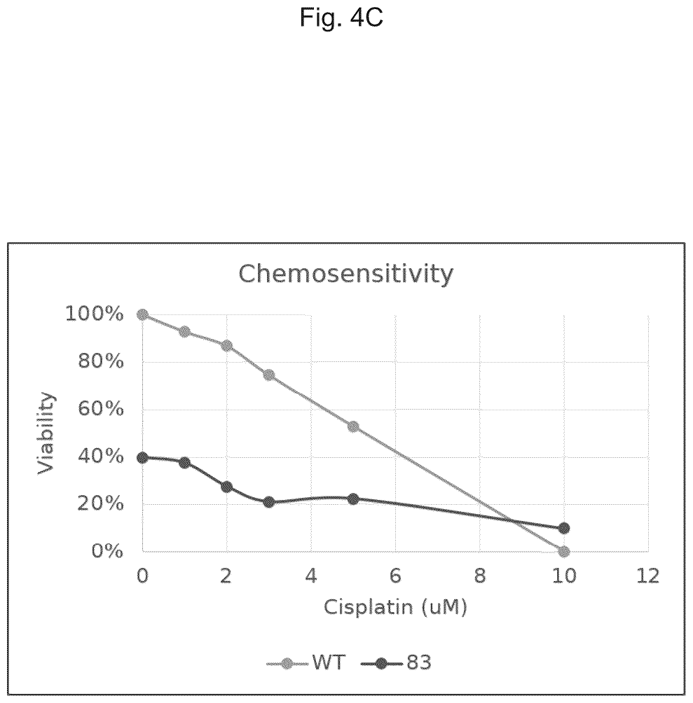

To assess CRISPR activity across various exons of NRF2, several gRNAs were designed along exon 3, 4 and 5 of the NRF2 gene in the A549 cell line. The A549 cells were transfected with each individually designed gRNA and cells were collected 72 hours after transfection. Genomic DNA was isolated, PCR amplified and Sanger sequenced. Editing activity was quantified using DECODR to deconvolute the indels present in at the cut site of each gRNA. FIG. 4A depicts a comparison of the total editing efficiency (Indel) and frameshifting efficiency (FS) of each gRNA (gRNA 73 (SEQ ID NO 9), gRNA 116 (SEQ ID NO 13), gRNA 4 (SEQ ID NO 5), gRNA 1 (SEQ ID NO 17), gRNA 2 (SEQ ID NO 34), gRNA 76 (SEQ ID NO 16), gRNA 77 (SEQ ID NO 18), gRNA 83 (SEQ ID NO 24), gRNA 94 (SEQ ID NO 35), gRNA 107 (SEQ ID NO 36)). FIG. 4B depicts the indel profile observed with gRNA 76 and gRNA 83, as deconvoluted by DECODR. Cells transfected with gRNA 83, which showed the highest editing efficiency, were used to assess cell viability after exposure to increasing concentrations of cisplatin. As shown in FIG. 4C, compared to the wildtype, untransfected cells (WT), the gRNA 83-transfected cells (83) showed much lower viability without any cisplatin exposure (0 uM). FIGS. 4C and 4D depict the deconvolution of indels by DECODR for cells transfected with gRNA 76 and gRNA 83.

### Example 4: Gene Knockout of NRF2 Increases Chemosensitivity of Human Glioblastoma (T98G & LN229) Cells

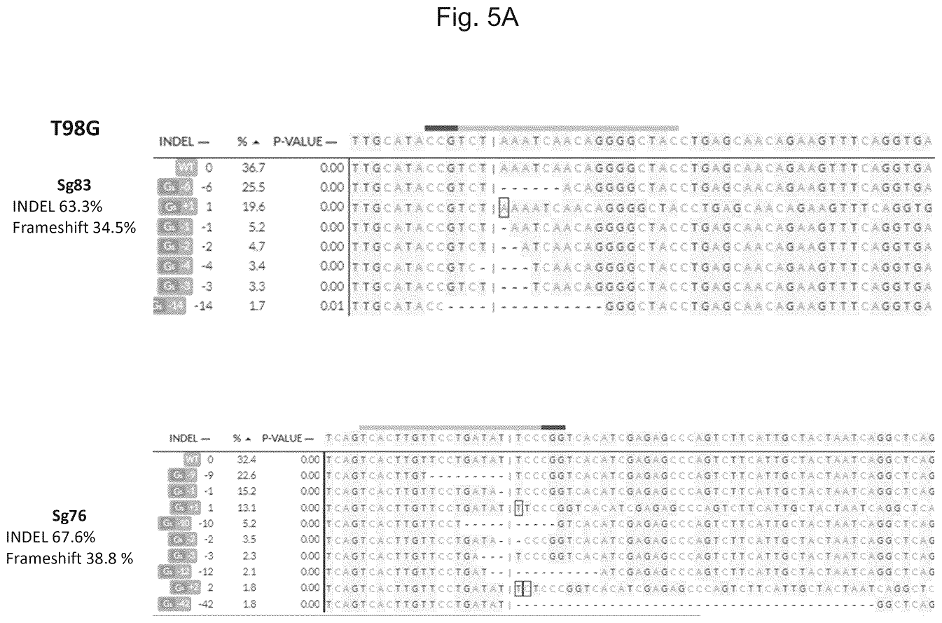

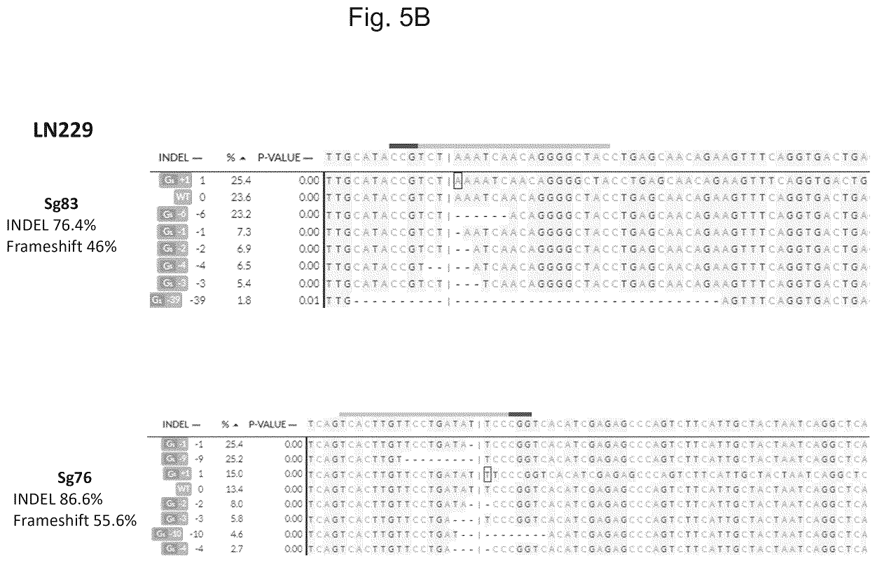

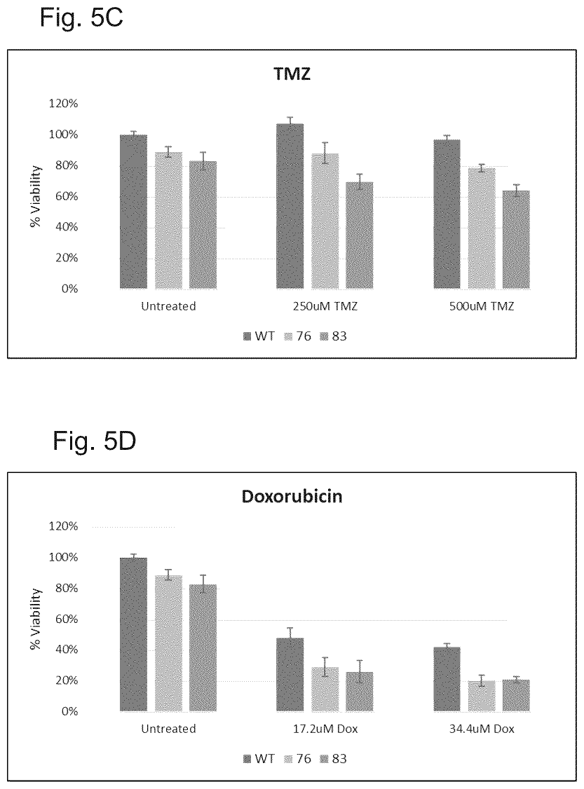

Two human glioblastoma-derived cell lines were used to assess CRISPR activity and downstream effects of NRF2 knockout. FIG. 5 compares targeting exon 4 by two sgRNAs (sg76 (SEQ ID NO:16) and sg83 (SEQ ID NO:24)) and chemosensitivity assessment after targeting in both glioblastoma T98G and LN229 cell lines. Both cell lines were transfected using CRISPRmax lipofection with each respective gRNA and Cas9 mRNA. Cells were collected 72 hours after transfection and genomic DNA was isolated, PCR amplified and Sanger sequenced. The DNA sequence data, as deconvoluted by DECODR, for T98G (FIG. 5A) and LN229 (FIG. 5B) indicates that each of the CRISPR molecules is efficient in knocking out NRF2 at exon 4. The editing efficiency for each respective gRNA in each respective cell line ranges from 63% to 87%. The targeted T98G cells were used to assess chemotherapy sensitivity after NRF2 knockout. With increasing concentrations of temozolomide (FIG. 5C) and doxorubicin (FIG. 5D), targeted with either gRNA 76 or gRNA 83, there is increased sensitivity to treatment. The gene knockout of NRF2 alone (untreated) is enough to decrease cell proliferation and viability.

### Example 5: Gene Knockout of NRF2 in Pancreatic Ductal Adenocarcinoma (Panc-1) Cells

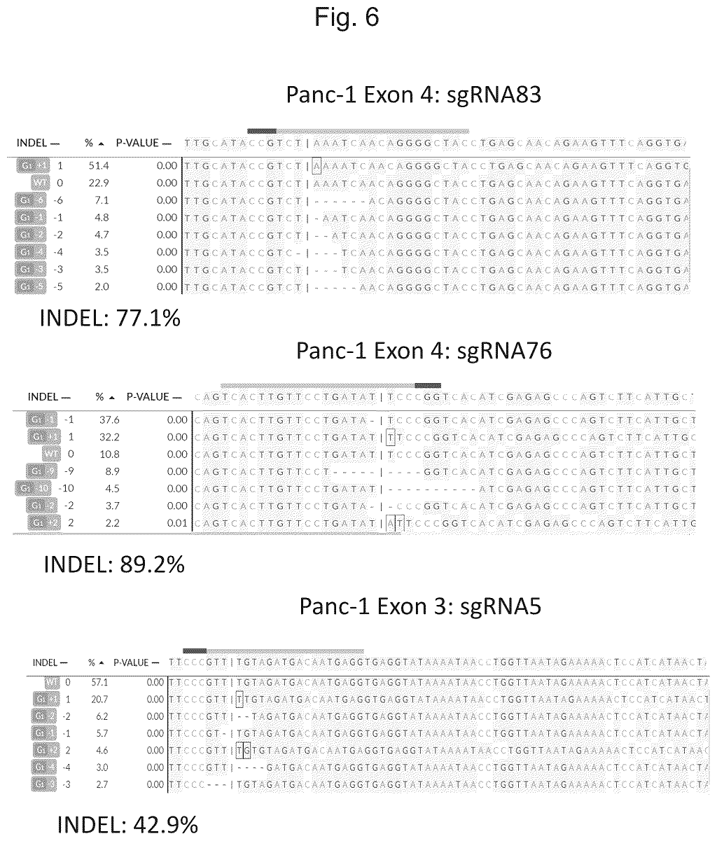

A human pancreatic ductal adenocarcinoma-derived cell was used to assess and compare CRISPR activity in the NRF2 gene. FIG. 6 compares targeting exon 4 and exon 3 using gRNA 76 (SEQ ID NO 16) and gRNA 83 (SEQ ID NO 24) and gRNA 5 (SEQ ID NO 10). Cells were transfected using Lonza nucleofection with each respective gRNA complexed with SpCas9 protein. Cells were collected 72 hours after transfection and genomic DNA was isolated, PCR amplified and Sanger sequenced. The DNA sequence data, as deconvoluted by DECODR, indicates high editing activity of each gRNA. The editing efficiency ranges from 42.9% to 89.2%.

### Example 6: LNPs are Effective Carriers of CRISPR/Cas Complex to Execute Significant Levels of Genetic Knockout in In Vivo Cancer Model

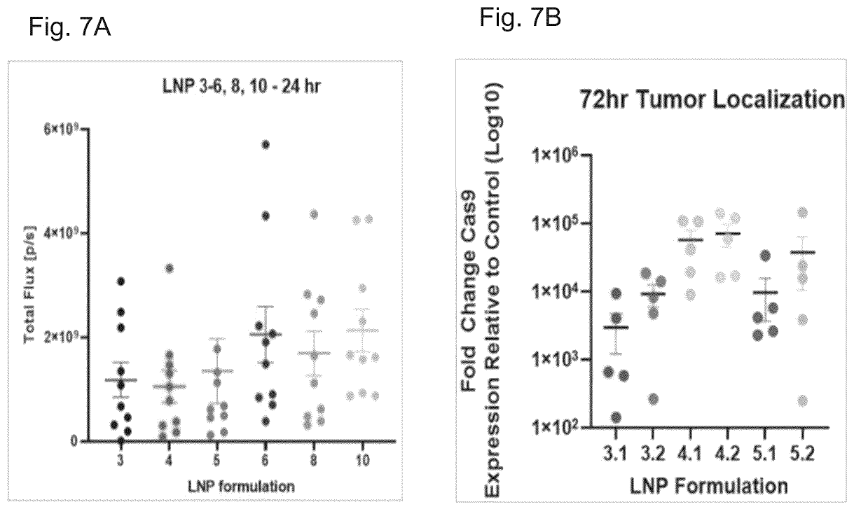

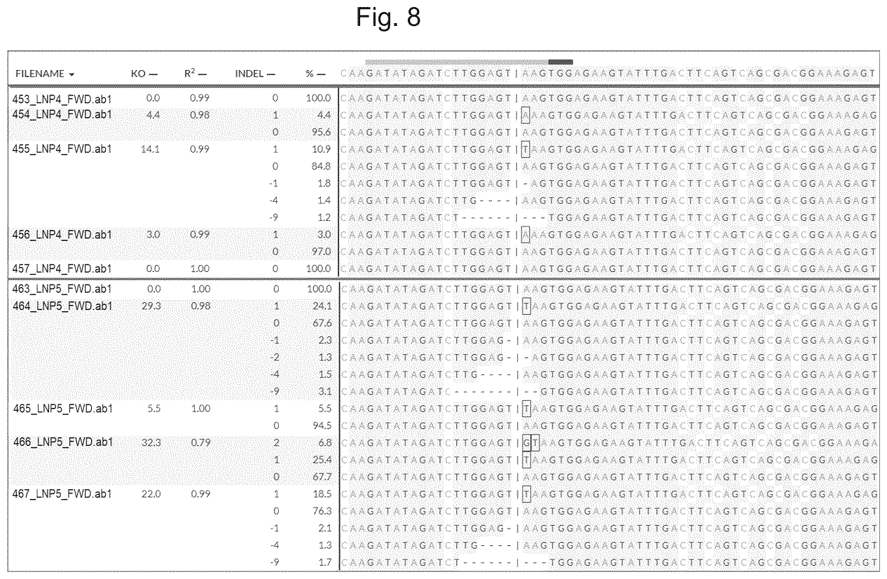

Different LNP formulations were used to assess intratumoral delivery of firefly luciferase or CRISPR/Cas9. To analyze the biodistribution of LNPs, luciferase expressing LNPs were injected in tumor-bearing xenograft mice. Twenty-four hours after LNP injection, bioluminescence imaging was performed with an IVIS Spectrum imaging system (Caliper Life Sciences). D-luciferin (Promega) was administered intraperitoneally at a dose of 150 mg/kg per mouse. Five minutes after receiving d-luciferin, mice were anesthetized in a chamber with 3% isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) (FIG. 7A) in the region of interest where bioluminescence signal emanated using the Living IMAGE Software provided by Caliper (Hopkinton, MA). To analyze localization of CRISPR/Cas9, LNPs encapsulating CRISPR/Cas9 were injected intratumorally in tumor-bearing xenograft mice. Seventy-two hours after injections, mice were sacrificed, and tumors were collected. Quantitative PCR was performed to detect the presence and localization of Cas9 mRNA. FIG. 7B displays the fold change expression of Cas9 of 3 different LNP formulations and their derivatives relative to the non-injected control. This indicates the LNP is sufficient to deliver the CRISPR/Cas payload to tumor cells. To assess gene editing activity after delivery of CRISPR LNPs, genomic DNA was isolated from tumors for sequencing. FIG. 8 displays the indel profiles detected and deconvoluted from Sanger sequencing of ten individual tumors. The editing efficiency, as determined by DECODR, ranges from 0-32.2% editing, confirming the presence of active CRISPR/Cas9 molecules within the tumor.

### Example 7 (Prophetic) Experimental Workflow to Assess Efficacy of CRISPR-Directed Knockout of NRF2 in Solid Tumors

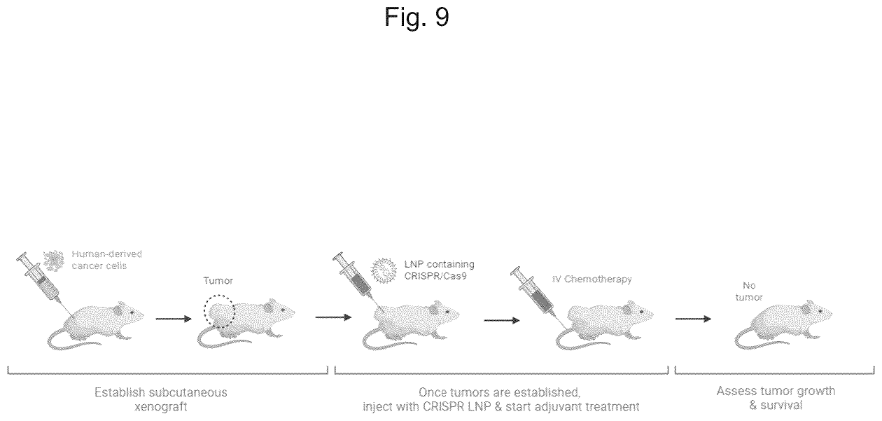

FIG. 9 presents the workflow of testing in vivo efficiency of CRISPR/Cas9 to reduce proliferation, migration and invasion or restore chemosensitivity. Human-derived cancer cells will be implanted subcutaneously in mice. Once tumors are established, CRISPR LNPS will be injected intratumorally. Standard of care adjuvant treatment will be used for each cancer indication. Tumor growth and overall survival will be assessed with and without adjuvant treatment.

To demonstrate NRF2 as a potential target to circumvent chemoresistance in solid tumors, cell-based xenograft model will be used to assess the functional outcome of NFR2 knock out. This will be evident from subsequent effectiveness to standard chemotherapy as a function of gene editing efficacy mediated by CRISPR/Cas technology. Preliminary studies in have demonstrated the potency of LNPs as a carrier, to effectively deliver the CRISPR/Cas9 payload to tumor cells, thus enabling the gene editing tool to function in vivo. This study will primarily involve phenotypic and genotypic analysis of tumor specimens to assess the efficacy of localized tumor delivery and functionality of the CRISPR/Cas machinery using any one of the guide RNAs listed in Table 1.

The effect of LNP CRISPR gene editing on the tumor in the absence of secondary treatment will be assessed. Tumor growth will be measured in all the mice every 2 days post injection of the formulation and tumor size will be estimated using our standard methodologies described above. After the designated period following treatment, mice will be sacrificed, tumors will be surgically removed and used for analyses.

Genomic DNA extracted from tumors will be PCR amplified in the region surrounding the CRISPR target site. The samples will be indexed and quantified, to calculate the average library size using the Tape station amplicon size. Once the libraries are pooled to the loading concentration, they are sequenced and only the data which passes QC in Sequencing Analysis Viewer will be analyzed using CRISPResso2 to produce and validate the gene editing efficiency. The editing efficiency/percent editing is defined as the total number of sequences reads with insertions or deletions over the total number of sequences reads, including wildtype.

Gene editing efficiency will be investigated for NRF2 gene knockout using the restoration of chemosensitivity to standard chemotherapeutic agents as a functional read out. Following the administration of LNP into the tumor as outlined above, gene editing will be allowed to take place for 24 hours. Chemotherapy will be administered by tail vein injection after 24 hrs. For each experiment, five animals will be used and the resulting change in tumor volume will be measured every two days for a period of two weeks or until control cohort needs to be sacrificed.

### Example 8 (Prophetic) Functional Outcomes of CRISPR-Directed Gene Editing of NRF2

Highly active gRNAs will be tested for their ability to disrupt the function of NRF2. Cells will be transfected with respective gRNAs, genomic DNA and RNA will be isolated and used for downstream analysis. Genomic DNA will be PCR amplified and Sanger sequenced to assess gene editing activity in cell population. RNA will be converted to cDNA and used in quantitative PCR to assess mRNA expression levels of downstream target genes of NRF2 such as NQO1, GCLC, GCLM, and HO-1.

### Example 9 (Prophetic): CRISPR-Directed Gene Editing of NRF2 Using Streptococcus pyogenes Cas9 Variant

CRISPR gRNA sequences (SEQ ID NO: 3-36) designed across exon 3, 4 and 5 will be tested for gene editing efficiency and impact on cell viability and chemosensitivity. Cells will be transfected with each individual gRNA. Genomic DNA will be isolated, PCR amplified and Sanger sequenced to assess gene editing activity. Transfected cells will be exposed to increasing concentrations of chemotherapy. Cell viability with and without chemotherapy treatment will be assessed using Cell-Titer Glo.

### Example 10 (Prophetic): CRISPR-Directed Gene Editing of NRF2 Using Staphylococcus aureus Cas9 Variant

CRISPR gRNA sequences (SEQ ID NO: 37-66) designed across exon 3 and 4 will be tested for gene editing efficiency and impact on cell viability and chemosensitivity. Cells will be transfected with each individual gRNA. Genomic DNA will be isolated, PCR amplified and Sanger sequenced to assess gene editing activity. Transfected cells will be exposed to increasing concentrations of chemotherapy. Cell viability with and without chemotherapy treatment will be assessed using Cell-Titer Glo.

### Example 11 (Prophetic): CRISPR-Directed Gene Editing of NRF2 Using Acidaminococcus sp. BV3L6 or Lachnospiraceae Bacterium Cas12a Variant

CRISPR gRNA sequences (SEQ ID NO: 67-74) designed across exon 3 and 4 will be tested for gene editing efficiency and impact on cell viability and chemosensitivity. Cells will be transfected with each individual gRNA. Genomic DNA will be isolated, PCR amplified and Sanger sequenced to assess gene editing activity. Transfected cells will be exposed to increasing concentrations of chemotherapy. Cell viability with and without chemotherapy treatment will be assessed using Cell-Titer Glo.

### Example 12: CRISPR-Directed Gene Editing to Disable NRF2 Functions to Restore Chemo-Sensitivity in Head/Neck and Esophageal Cancer Cells

### Cell Line and Culture Conditions

Human esophageal squamous cell carcinoma KYSE-410 cells and hypopharyngeal squamous cell carcinoma FaDu cells were purchased from MilliporeSigma (Burlington, MA, USA) and ATCC (Manassas, VA, USA) respectively. Cells were thawed, according to the manufacturer's protocol. KYSE-410 cells (KYSE, hereafter) were cultured in RPMI medium augmented with 2 mM Glutamine and 10% fetal bovine serum (FBS). FaDu cells were grown in Eagle's Minimum Essential Medium (EMEM) medium supplemented with 10% FBS and grown at 37° C. in 5% CO2. Cell lines were evaluated for Mycoplasma upon thawing and before use in experiments using the MycoScope PCR Mycoplasma detection kit (AMSBIO, Cambridge, MA).

### CRISPR/Cas9 Design

The NRF2 gene-coding sequence was entered into SnapGene and the following gRNAs were selected for targeting exon 2: (sg3) 5′-TATTTGACTTCAGTCAGCGA-3′ (SEQ ID NO:75); as well as two gRNAs targeting Exon 4: (sg76) 5′-GTCACTTGTTCCTGATATTCCCGG-3′ (SEQ ID NO:16), (sg83) 5′-GTAGCCCCTGTTGATTTAGACGG-3′ (SEQ ID NO:24). Based on the gRNA design, synthetic single gRNAs were ordered from Synthego (Menlo Park, California, USA). CleanCap® Cas9 mRNA (5moU) was ordered from TriLink Biotechnologies (San Diego, CA, USA).

### Lipofectamine MessengerMAX Transfection

Either one million cells were seeded to a 75 cm² tissue culture flask or 2.5 million cells were seeded to a 175 cm² tissue culture flask 24 hours prior to transfection and allowed to reach 60-80% confluency. On the day of the transfection, cells were treated with Lipofectamine MessengerMAX reagent (Thermo Fisher Scientific, Waltham, MA) bearing 10 μg or 25 μg of Cas9mRNA and 10 μg or 25 μg of sgRNA, suspended in Opti-MEM, per the manufacturer's instructions. 39 μL or 97.5 μL of MessengerMax lipofectamine reagent was diluted in 1.5 mL or 3.75 mL OPTI-MEM medium and vortexed twice was incubated for ten minutes at room temperature, during which time the culture media was replaced with 15 mL or 25 mL of fresh media, as appropriate to the cell line. A total of 20 μg or 50 μg of RNA was then added to the diluted lipofectamine, consisting of 10 μg or 25 μg Cas9 mRNA and 10 μg or 25 μg sgRNA. The suspension was incubated at room temperature for an additional five minutes before being added, in full, to the tissue culture flask, which was then swirled gently to disperse the reagent. Treated cells were incubated for 72 hours at 37° C. prior to being assayed.

### Next Generation Sequencing Gene Editing Analysis

Targeted amplicon libraries were prepared using Illumina's 16S metagenomic sequencing library preparation protocol for MiSeq. Genomic DNA was extracted from cells using the Qiagen's DNeasy Blood & Tissue kit (Quiagen, Hilden, Germany) or QuickExtract reagent (Biosearch Technologies). The region surrounding the CRISPR target site for Exon 2 (398 bp) and Exon 4 (402 bp) of the NRF2 gene were amplified using the 2× Phusion Flash PCR master mix (Thermo Fisher Scientific, Waltham, MA). Post amplification, samples were cleaned using AMPure beads and DNA concentration was measured using the Qubit assay (Thermo Fisher Scientific, Waltham, MA). Samples are also run on TapeStation (Agilent Technologies Santa Clara, CA) to verify the presence of the desired amplicon. The samples were indexed using either the IDT for Illumina DNA/RNA UD indexes set A (Illumina San Diego, CA). Samples were again quantified using Qubit and average library size was calculated using the Tapestation amplicon size. Once pooled to the loading concentration, the libraries were sequenced using the MiSeq Reagent kit v2 (Illumina, San Diego, CA). Only the data that passed data QC in Sequencing Analysis Viewer, was analyzed using CRISPResso2 (CRISPResso2 website) to understand gene editing efficiency and frameshift analysis.

### Cell Viability

Transfected and wildtype KYSE and FaDu cells were plated in 24-well plates (n=4) at 50,000 cells per well and incubated for 24 hours. The cells were then treated with one of: 0, 2.5, 5, 15, or 30 μM cisplatin (a gift from ChristianaCare Pharmacy) or with 100, or 300 μM 5-Fluorouracil (Selleck, Houston, TX) for 3 days. Cell viability after drug exposure was evaluated using the CellTiter-Glo 2.0 Cell Viability Assay (Promega, Madision, WI). CellTiter Glo reagent was added to wells at 1:1 volume with the cell suspension. The plate was covered in foil and placed on an orbital shaker for 2 minutes before being removed. The plate was allowed to equilibrate at room temperature for another 10 minutes before the luminescence of its contents was measured using an Infinite 2000 PRO microplate reader (Tecan, MAnnedorf, Switzerland). The data was plotted with the coefficient of variance.

### RT-PCR

Total RNA was isolated from edited cells using TRIzol reagent and PureLink RNA Mini Kit (Thermo Fisher Scientific, Waltham, MA). Reverse transcription was conducted using the Applied Biosystems High-Capacity RNA-to-cDNA kit (Thermo Fisher Scientific, Waltham, MA). The cDNA was used as the template in the qPCR amplification of GAPDH (Fwd 5′ TCTCCTCTGACTTCAACAGCGAC3′ (SEQ ID NO:76), Rev 5′CCCTGTTGCTGTAGCCAAATTC3′ (SEQ ID NO:77)), NRF2 (Fwd 5′TCCAAGTCCAGAAGCCAAACTGAC3′ (SEQ ID NO:78), Rev 5′GGAGAGGATGCTGCTGAAGGAATC3′ (SEQ ID NO:79)), NQO1 (Fwd 5′GGTTTGGAGTCCCTGCCATT3′ (SEQ ID NO:80), Rev 5′TTGCAGAGAGTACATGGAGCC3′ (SEQ ID NO:81)), HMOX1 (Fwd 5′ CTTTCAGAAGGGCCAGGTGA3′ (SEQ ID NO:82), Rev 5′GTAGACAGGGGCGAAGACTG3′ (SEQ ID NO:83)) and GCLC (Fwd 5′GGACAAGAATACACCATCTCCA3′ (SEQ ID NO:84), Rev 5′ ATACTGCAGGCTTGGAATGTC3′ (SEQ ID NO:85)) transcripts using the SsoAdvanced universal SYBR Green Supermix (BioRad, Hercules, CA). The experiment was conducted at least twice with all samples run in duplicate at minimum. Relative expression levels of mRNA were calculated using the Livak method and normalized to the control. The data was plotted with the coefficient of variance.

### Exon Skipping Analysis

Total RNA was isolated from edited cells using TRIzol reagent and PureLink RNA Mini Kit (Thermo Fisher Scientific, Waltham, MA). Reverse transcription was conducted using the Applied Biosystems High-Capacity RNA-to-cDNA kit (Thermo Fisher Scientific, Waltham, MA). The cDNA was used as the template for PCR amplification with either Phusion Flash or Phusion High-Fidelity PCR master mix (Thermo Fisher Scientific, Waltham, MA) with primers spanning the 5′UTR region through Exon 5 (798 bp) of the NRF2 gene. PCR reactions were purified using Qiagen Qiaquick PCR cleanup columns (Quiagen, Hilden, Germany) according to manufacturer's protocol, measured on a Nanodrop and equally loaded on a 1% agarose gel.

### Protein Preparation and Western Blotting

Total protein was isolated from cells in RIPA buffer (Pierce), incubated on ice for 30 minutes with vortexing every 10 minutes. Samples were centrifuged at 14,000×G at 4° C. for 15 minutes and the supernatant was saved. Protein extracts were quantified using a BCA assay (Pierce) and 20 μg of protein was loaded onto a Biorad 4-20% polyacrylamide pre-cast gel (Biorad). Protein was transferred onto a 0.2 μM nitrocellulose blot using the Turboblot dry transfer system (Biorad). The membrane was blocked in 5% milk in TBS-T for 2 hours at room temperature and stained with anti-NRF2 1:1000 (Abcam, ab62352) and anti-GAPDH 1:5,000 (Cell Signaling Technology, 97166). Blots were washed 3× at room temperature in TBS-T and stained with secondary antibodies conjugated to HRP 1:10,000 (Abcam, ab205718 or Thermofisher, PI31430). Blots were imaged using the Pierce Femto western blotting substrate (Pierce). Bands were quantified using the FIJI gel analysis tool package.

### The Experimental System and Target Sites

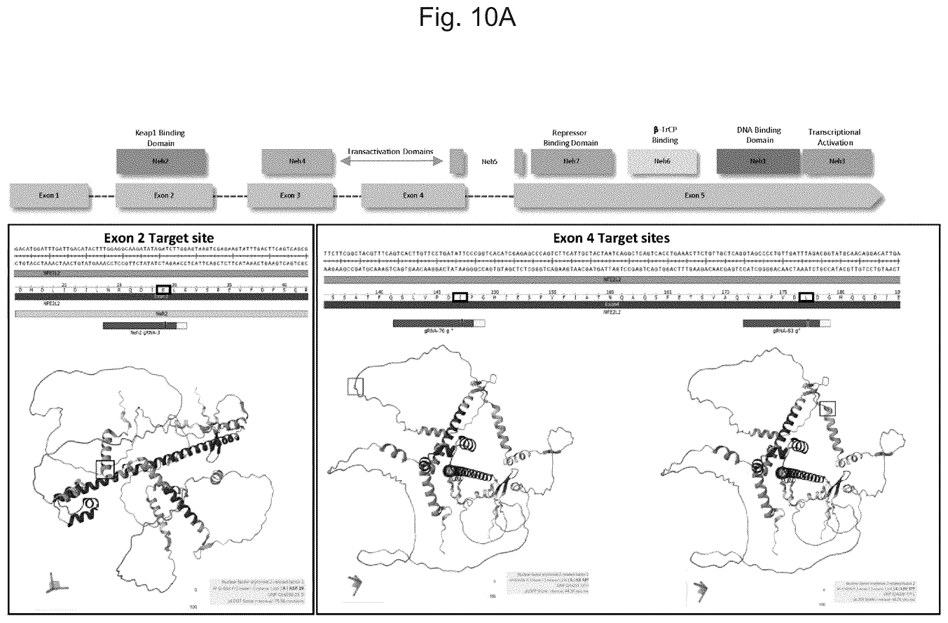

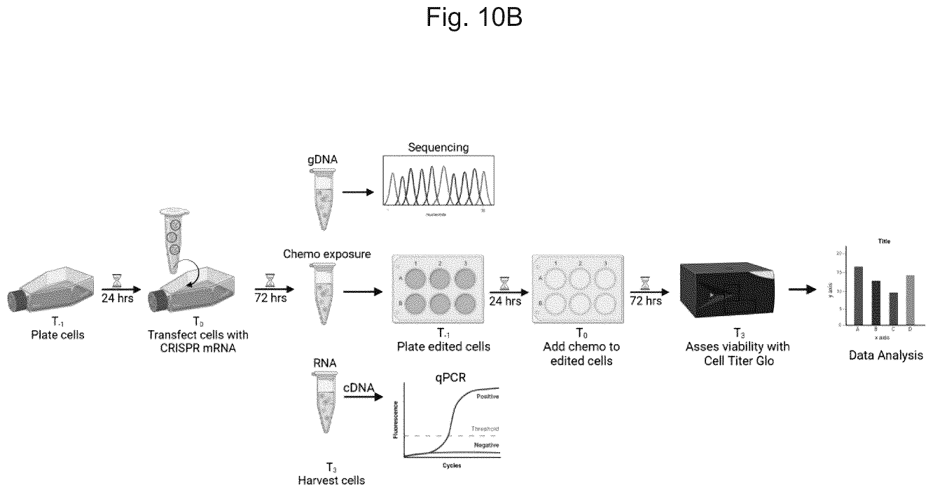

The NRF2 gene is segmented into five exons, each encoding important functional protein domains (FIG. 10A). Exon 2 is well known because it is the domain (Neh2) that enables the interaction with KEAP 1²⁸⁻³⁰. Exon 4 links the Neh4 and Neh5 domains which are critically important for the overall function of NRF2 in its role as a master regulator and transcription factor. We have previously demonstrated that both exons can be targeted by CRISPR/Cas resulting in the de-activation of NRF2 gene function in adenocarcinoma and squamous cell carcinoma of the lung³¹⁻³³. Exon 2 harbors a unique mutation, R34G, that is found in a subset of squamous cell patients, and this mutation creates a unique PAM site enabling tumor-specific CRISPR activity^{32,34,35}. Exon 4 has also been exploited as a target site with initial disruption site in A549 lung cancer cells severely reducing NRF2 activity³¹.

The objective of this study is to evaluate the functional outcomes of CRISPR-directed gene editing through the disruption of NRF2, and the restoration of sensitivity to chemotherapy in squamous cell carcinoma of the Head and Neck. Previous results guided our decision to create sgRNAs that target either exon 2 or exon 4, for disruption of NRF2 in head and neck cancer cells. Unlike the case of squamous cell carcinoma of the lung, the R34G mutation exists in head and neck cancer cells at a lower frequency than in the lung (28). So, here we targeted a region of normal NRF2 sequence, sg3 (TGGAGGCAAGATATAGATCT (SEQ ID NO:86)) at the DLG motif designed to disrupt exon 2. In parallel, two guide RNAs, sg76 (SEQ ID NO:16) and sg83 (SEQ ID NO:24) were designed to target exon 4. Sg3 and sg83 target alpha loop structures of the NRF2 protein, while sg76 targets a disorganized region.

FIG. 10B outlines the work plan carried out on FADU cells which were established from a punch biopsy of a hypopharyngeal tumor removed from a 56-year-old, White, male patient with squamous cell carcinoma³⁶. Cells were plated 24 hours prior to the introduction of the CRISPR/Cas complex, with the respective sgRNA, and delivered into the cells by the Lipofectamine™ MessengerMAX mRNA Transfection Reagent. Seventy-two hours after exposure to CRISPR/Cas, a sample of the cells was harvested for genomic sequencing and RNA isolation while the remaining population was replated and recovered for 24 hours. After that time, the cell population was treated with Cisplatin or 5-fluorouracil (5-FU) at various doses. Cisplatin and 5-FU were selected based on NCCI treatment guidelines as Cisplatin is the standard chemotherapy drug, clinically recommended as part of chemoradiation alone or in combination with 5-FU³⁷⁻³⁹. Three days after treatment, the cells were harvested, and cell viability was measured. This conservative workflow was established to assess how gene editing taking place prior to the introduction of the chemotherapeutic agent drives the restoration of sensitivity.

This experimental plan allows for more direct analyses of the impact of CRISPR activity, which we recognize could continue in the presence of chemotherapy. We decided to use a single dose of the drug at a single point in time, a conservative approach to drug testing in cell culture, although multiple rounds of chemotherapy at lower therapeutic doses post-gene editing could be more effective if translated into the clinical setting.

For all gene editing studies from bench to bedside, it is important to employ a functional assay, accompanied by an internal functional control, to assess the efficiency of CRISPR/Cas activity. In this case, NRF2 is a transcription factor that controls the expression of over 200 genes. Thus, NRF2 disruption can be measured functionally through qPCR analysis of NQO1 expression, a keystone gene controlled by NRF2. NQO1 has been used as a standard measure of NRF2 activity in cancer cells⁴⁰. We also use downstream targets HMOX1 and GCLC, two major antioxidant enzymes, to assess NRF2 function.

### Genetic Disruption of NRF2 at Exon 2 in FADU Cells

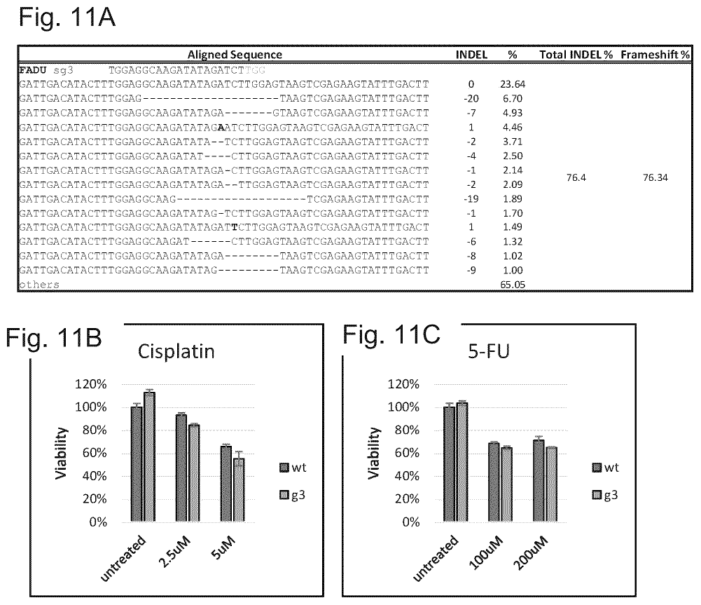

FIG. 11A displays the indel profile generated by the sgRNA 3/Cas9 complex after 72 hours of incubation. The total indel population exceeded 76% and more importantly, within that profile, over 76% (of the 76% total) were frameshift mutations. The overall indel profile is less important than the population of frameshift mutations occurring on either or both alleles within that profile, a hierarchy we have termed, the Indel Code. Under any conditions, inducing allelic knockout of 76% in FADU cells reflects high levels of gene editing. However, despite high levels of genetic disruption, targeted cells were rendered minimally chemo-sensitive FIG. 11B-C and retained the same level of drug resistance as untreated cells. Importantly, cells that did not receive chemotherapeutic drugs, were treated exactly as those that had, including mock transfection and recovery. These results were surprising considering genetic disruption at the NRF2 R34G gene mutation in exon 2 resulted in significant restoration of chemosensitivity in squamous cell carcinoma of the lung^{32,35}.

### Genetic Disruption of NRF2 at Exon 4 in FADU Cells

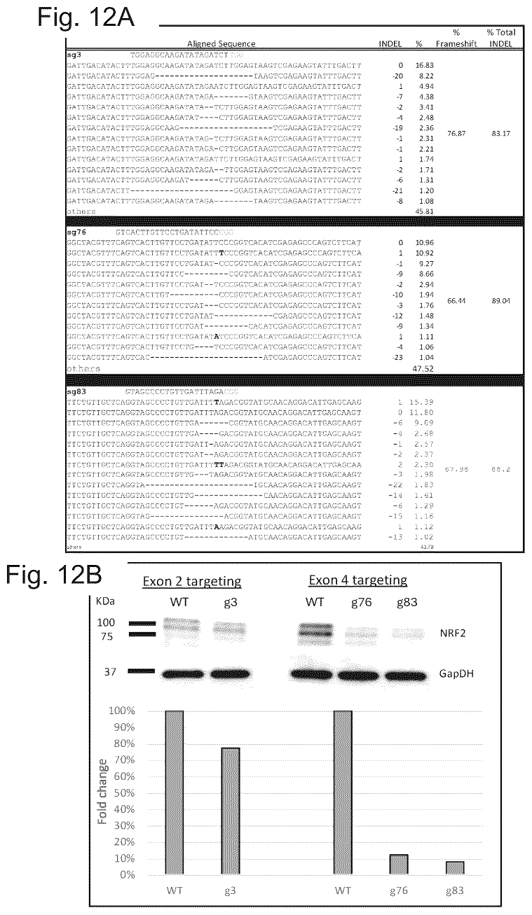

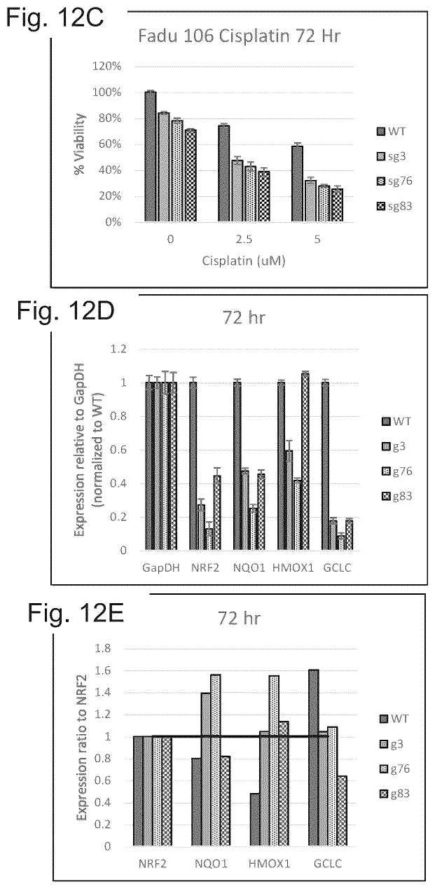

Following the unexpected results of NRF2 exon 2 disruption with sg3, we repeated the experimental protocol using two different guides, sg76 and sg83, which target exon 4. Once again, gene editing was assessed after 72 hours and the indel profiles are presented in FIG. 12A. The degree of NRF2 gene disruption paralleled levels seen with sg3 and the percentage of frameshift (Indel Code) was also similar. NRF2 is also knocked down at the protein level at the 72-hour timepoint (FIG. 12B). Exon 2 sg3 shows 30% knock down, while 90% knock down was achieved by targeting Exon 4 with sg76 and sg83. We then added Cisplatin for 72 hours and measured the viability of the targeted and the untargeted cells. FIG. 12C displays the results. Untargeted cells, or those without the addition of Cisplatin, were processed the same as targeted cells treated with the drug. FADU cells edited by either sg76 or sg83, followed by a single dose of Cisplatin, exhibited a significant reduction in viability and, therefore restoration of chemosensitivity. We also observed that exon 4 edited cells showed reduced viability from just the disruption of NRF2 alone compared to the exon 2 targeted. These data suggest that the choice of the target site within NRF2 elicits significant functional disruption. In addition, (FIG. 12D) NQO1 and GCLC expression levels were diminished when FADU cells were transfected with sg83. To better understand NRF2 function in each knockdown condition, we divided the relative expression from FIG. 12D by the NRF2 knockdown level in each guide condition to produce a ratio of NRF2 expression to each downstream target (FIG. 12E). While NRF2 levels are largely reduced in all 3 targeted conditions, g3 and g76 do not show a reduction in expression of downstream target genes relative to NRF2 levels. This analysis provides further evidence to support the impact of genetic disruption alone of NRF2 is sufficient to disrupt its transcription activation function.

### Genetic Disruption of NRF2 at Exon 2 or Exon 4 in KYSE-410 Cells

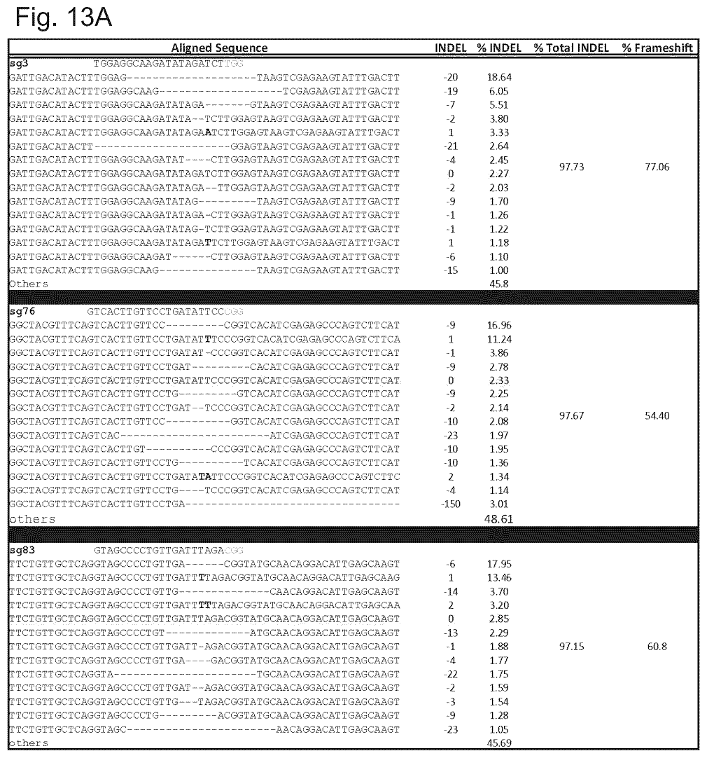

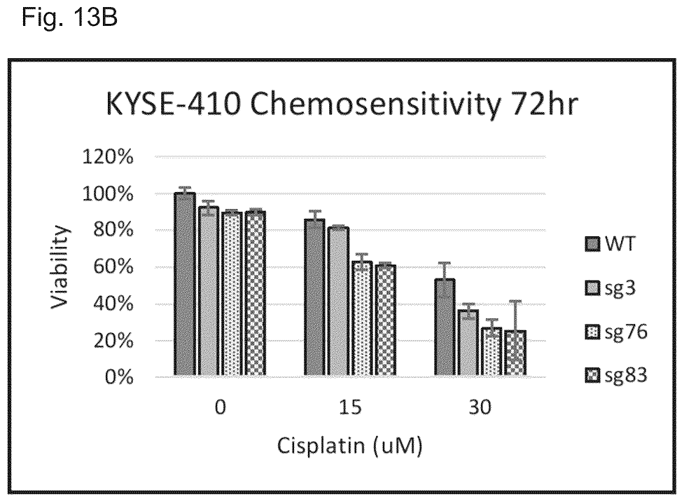

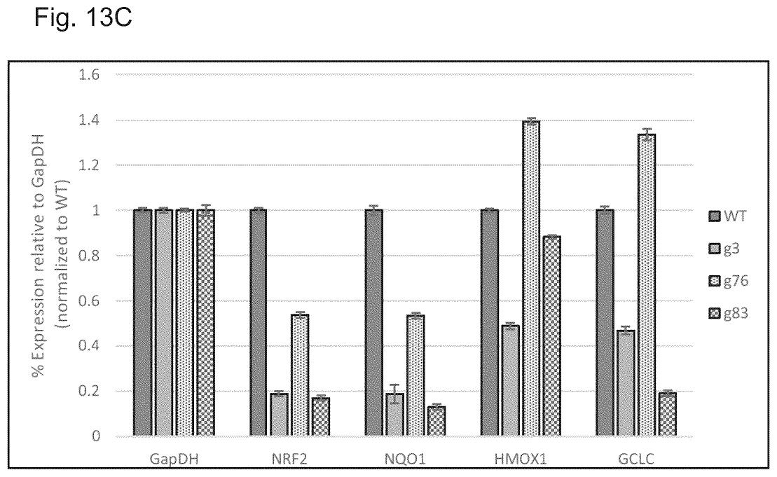

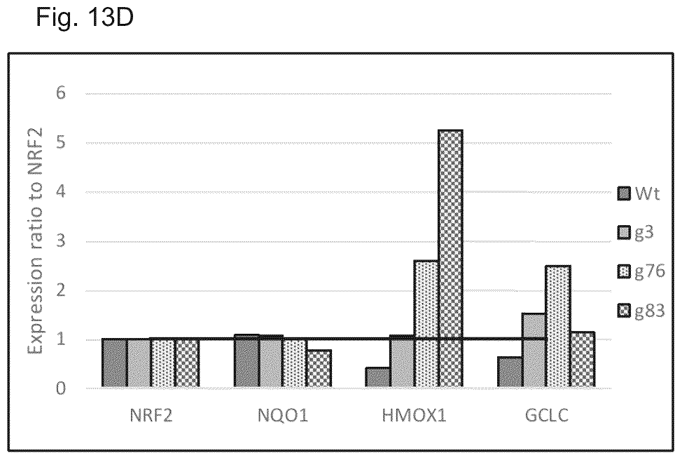

To evaluate this approach for broad based applications, we executed the same protocol on KYSE-410 cells which originate from the poorly differentiated invasive esophageal squamous cell carcinoma resected from the cervical esophagus of a 51-year-old Japanese man prior to treatment⁴¹. First, we confirmed that the DNA sequence of exon 2 and exon 4 was identical and amenable to targeting with CRISPRs sg3, sg76, or sg83 respectively (data not shown). While this may seem like a trivial matter, it is not, particularly with mutator phenotypes in transformed cells. Plated cells were allowed to recover for 24 hours prior to transfection with the same CRISPR complexes as described in FIG. 12. Seventy-two hours post-transfection, the indel profiles revealed a high degree of gene editing activity approaching 100% (97%) with all three guides, sg3, sg76 and sg83 (FIG. 13A). These overall levels are generally considered to be a complete genetic disruption of a mammalian gene. The percentage of frameshift mutations created by the action of these guide RNAs mimics those levels seen previously in FADU cells. The response of targeted cells to Cisplatin reflected the levels of chemosensitivity restored by sg76 and sg83, but importantly, not by sg3 (FIG. 13B). As previously reported, cisplatin concentrations were adjusted to account for the relative resistance in KYSE-410 cells⁴². The expression levels of NRF2, NQO1, HMOX1, and GCLC were also assessed, FIG. 13C. Exon 4 targeting by sg83 results in lower levels of NQO1 and GCLC expression than the exon 2 (sg3) or exon 4 (sg76) targeted cells. To better characterize these results the ratio of NRF2 expression to each downstream target was calculated, FIG. 13D. This analysis further supports that disruption of NRF2 in exon 4 has a higher impact on overcoming chemosensitivity by disrupting its function of activating downstream genes.

These results collectively emphasize the importance of the choice of the genetic targeting site and the domain of the encoded protein to achieve the desired phenotypic and functional outcome. In addition, the internal consistency among the editing activity of the specific guide RNAs, where disruption was achieved above 85% in both FADU and KYSE 410 cells, codifies the identification of sg76 and sg83 as useful biomolecules for disruption of NRF2 at all levels.

Exon skipping can regulate functional outcomes of NRF2 gene disruption.

Genomic modification at unintended sites is at the heart of the safety concerns surrounding CRISPR-directed gene editing. While much emphasis is placed on off-site mutagenesis, more often, intragenic events, including on-site alterations such as genomic translocations, inversions, gene conversion, and unexpected disruption of mRNA sequence^{43,44}, are likely more impactful.

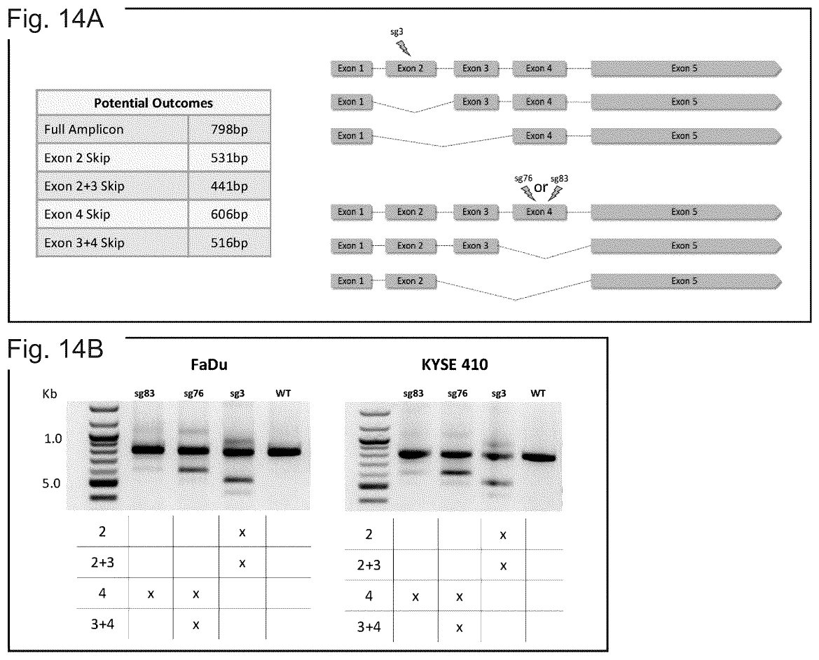

The difference between targeting exon 2 and exon 4 was intriguing, so we wanted to see if a molecular rearrangement, exon skipping, which we had identified previously within the NRF2 gene was at least in part responsible for this difference³³. Since we know NRF2 exon 2 and exon 4 are disrupted by design, and intra-genic changes are the objective of the experiment, we asked if exon skipping could account for the differential response in targeting exon 2 versus exon 4. FIG. 14A represents the potential exon skipping outcomes. For both the FADU and the KYSE-410 cells, at 72 hours post-transfection RNA was converted to cDNA followed by PCR amplification of the NRF2 cDNA (798 bp) spanning the 5′UTR to exon 5. The results in FIG. 14B show that exon skipping occurs, predominantly when sg3 and sg76 are used to target exon 2 and exon 4 respectively. Full length mRNA (Wt.) shows a defined band at the expected size of 798 bp. But both cell lines targeted with sg3 and sg76, present a second lower band indicative of an exon skipping event. The 798 bp is the full transcript while the lower band for sg3 is 531 bp and sg76 is 606 bp, the expected sizes of the removal of exon 2 (267 bp) and exon 4 (192 bp). In the case of exon 4 sg83 targeted KYSE-410 and FaDu cells some level of exon skipping activity is detected but at much lower rate than with sg3 or sg76.

### Sustainability of the Genetic and Functional Disruption of NRF2

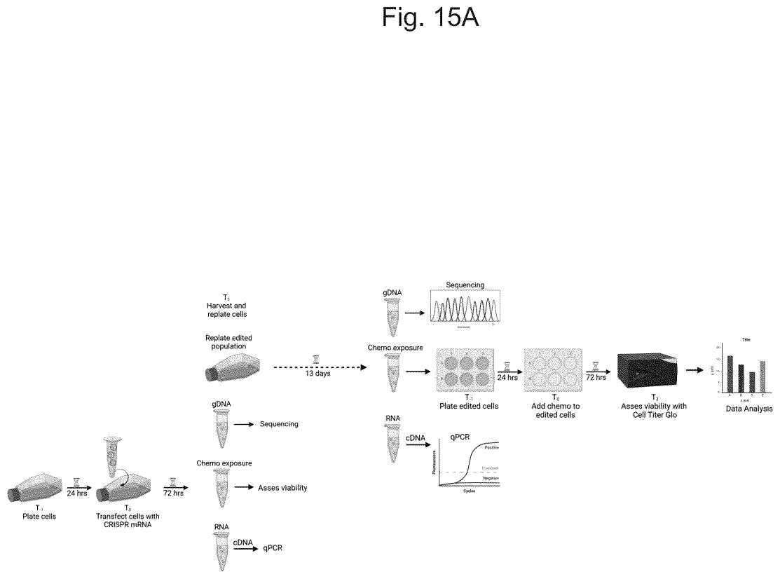

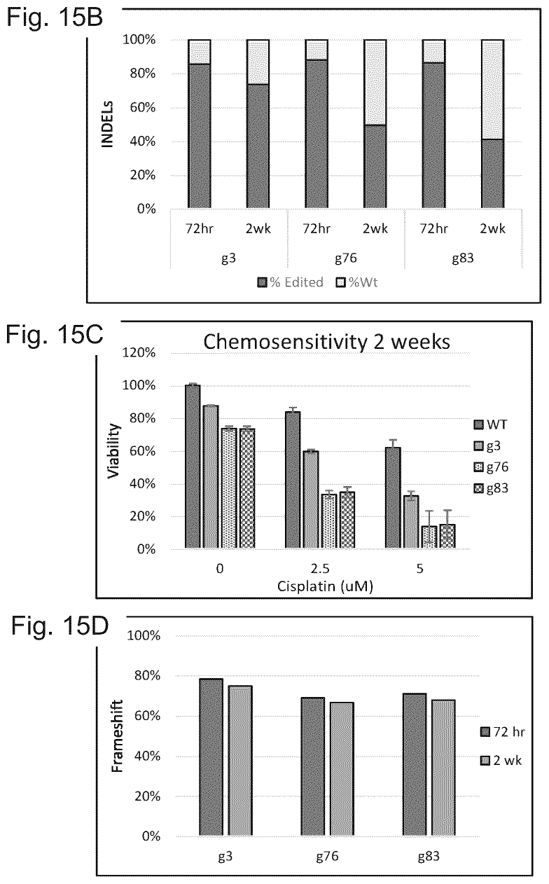

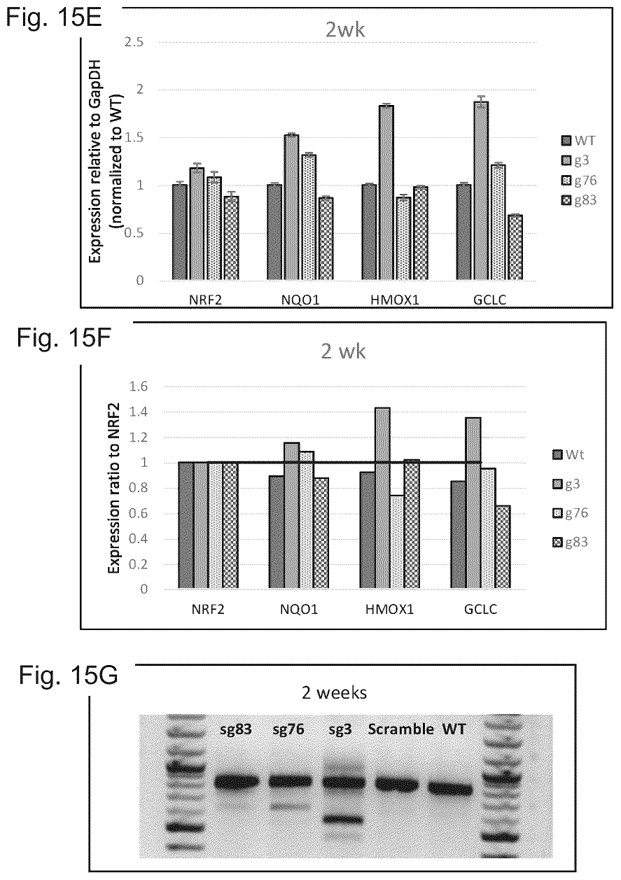

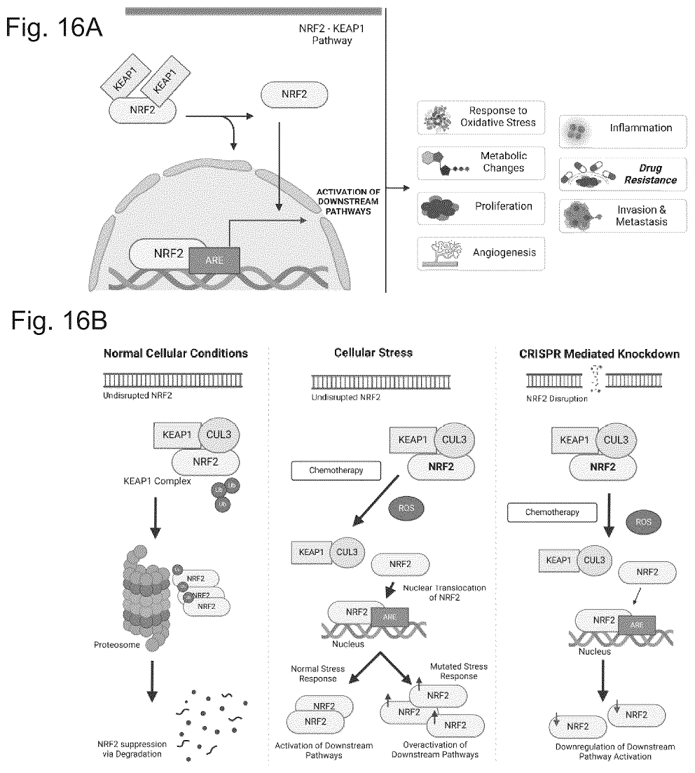

Next, we were interested in assessing whether the targeted cells maintained functional disruption of NRF2 over time. We followed the same experimental workflow, but extended cell growth for 13 days (FIG. 15A). At this time, cells were collected for gDNA sequencing, RNA isolation, while remaining cells were plated and analyzed for chemosensitivity. After two weeks, the exon 2 (sg3) targeted population of cells maintained a significant level of gene editing exhibiting 73.45% editing (72 hr: 85.5%) (FIG. 15B). However, the percentage of exon 4 targeted cell populations bearing edited cells diminished slowly as a population of unedited cells expanded steadily. Exon 4 sg76 exhibited 49.30% (72 hr: 87%) and sg83 exhibited 40.95% (72 hr: 86%) (see FIG. 15B). Interestingly, despite the reduction of INDELs in the cell population, the cells maintained their hypersensitivity to Cisplatin, despite the growth of the unedited cell population exon 4 targeted still sustained higher sensitivity than the exon 2 targeted (FIG. 15C). From a genomics standpoint, an interesting observation emerges; although there was a reduction in total INDEL percent after two weeks, the percentage of cells bearing frameshift mutations within those populations remained the same (FIG. 15D). Given the sustained chemosensitivity despite the change in the edited cell population, we assessed the status of NRF2 function on its downstream genes NQO1, HMOX1, and GCLC via qPCR (See FIG. 15E/F). As seen in the 72-hour analysis (FIG. 12C) exon 4 sg83 results in lower expression of NQO1 and GCLC compared to sg3 and sg76. In the case of exon 2 sg3 the opposite effect is seen where there is an increase in expression for all assessed genes NQO1, HMOX1, and GCLC. These results could be attributed to the effects captured over time of disrupting the two different functional domains of NRF2 acting in opposite functions. Exon skipping was also examined with no substantial changes in the results observed at 72 hours (FIG. 15G). These results confirm that the functional outcomes are sustainable and could provide some insight into the importance of the type of indels that are most effective in producing those outcomes (Indel Code).

### Example 14. CRISPR-Directed Gene Editing to Augment Systemic Therapy for Pancreatic Ductal Adenocarcinoma

### Cell Line and Culture Conditions

Human pancreatic ductile adenocarcinoma cells (PANC-1) were purchased from ATCC (Manassas, VA, USA). Cells were thawed according to the manufacturer's protocol. PANC-1 cells were cultured in Dulbecco's Modified Eagle's Medium (DMEM) (ATCC) supplemented with 10% FBS and grown at 37° C. in 5% CO2.

### CRISPR/Cas9 Design

The NRF2 gene-coding sequence was downloaded into a SnapGene file and then the Synthego (Menlo Park, California, USA) CRISPR design tool was used to select sequences across the NRF2 gene in exon2, exon3, exon 4, and exon5. The sequences were uploaded into the SnapGene file, and two gRNA sequences were selected for testing (Table 2). Synthetic single gRNAs were ordered from Synthego (Menlo Park, California, USA) using the gRNA designs. SpCas9 2NLS Nuclease (1000 pmol) was also ordered from Synthego (Menlo Park, California, USA).

TABLE 1
sgRNA sequences and exon target

| | | | |
|---|---|---|---|
| Exon 3 | sgRNA3 | 5′-CCTCATTGTCAT | SEQ ID NO: 87 |
| | | CTACAAACGGG-3′ | |
| Exon 4 | sgRNA83 | 5′-GTAGCCCCTGTT | SEQ ID NO: 24 |
| | | GATTTAGACGG-3′ | |

### RNP Nucleofection Transfection

Three million cells were seeded to a 75 cm² tissue culture flask 24 hours prior to transfection and allowed to reach 60-80% confluency. On the day of transfection, RNP was complexed using sgRNA and spCas9 at a 5:1 (250:50 pmol) ratio and left to incubate at room temperature for 15 minutes. While RNP incubated, cells were harvested, and one million cells were placed into a 1.5 mL tube. The cells were spun down at 300×g for 5 minutes, and media was aspirated from tube before cells were resuspended in 1 mL PBS. The cells were then spun down again, and PBS was aspirated from tube before cells were resuspended in 100 μL Lonza SE solution. 5 μL of RNP complex was added to resuspended cells and cells were transfected using Lonza program EO-137, cells were resuspended in 500 μL pre-adapted media and left in incubator for 10 minutes, cells were then plated to T25 flask and left to recover for 24-72 hours.

### Sanger Sequencing Gene Editing Analysis

Genomic DNA was extracted from harvested cells using the Qiagen DNeasy Blood & Tissue Kit (Cat. 69504). Amplicons were designed to encompass the CRISPR target site for each exon within the NRF2 gene, exon 3 (517 bp) and exon 4 (402 bp) were then amplified using Q5®High-Fidelity 2X Master Mix (New England Biolabs, cat. M04292). The samples were cleaned up post-amplification using the QIAquick PCR Purification Kit (Cat. 28106). The BigDye™Terminator v3.1 Cycle Sequencing Kit (Thermo Fisher, Cat. 4337455) was then to run a secondary PCR reaction using 20-20 μg of purified genomic DNA from PCR 1 and the primer sets used in the initial reaction to amplify exons respectively. Once the BigDye™ Terminator run was complete samples were cleaned using the BigDye XTerminator™ Purification Kit (Thermo Fisher, Cat. 4376486) and placed into the SeqStudio platform for analysis and readout. Readout was analyzed post sequencing using DECODR™ v 3.0 software (ChristianaCare Gene Editing Institute, Delaware, USA).

```
Exon 3 Forward Primer:
  (SEQ ID NO: 88)
5′-GTGGTCTAGTTCAAATTGTGC-3′
Exon 3 Reverse Primer:
  (SEQ ID NO: 89)
5′-GGTTATGCTGTCCATGTTTC-3′
Exon 4 Forward Primer:
  (SEQ ID NO: 90)
5′-GTAGTGGTGCCTTAGAGCTTACTCATCC-3′
Exon 4 Reverse Primer:
  (SEQ ID NO: 91)
5′-CTAGCATGGGCAGTACTCATGACTAAG-3′
```

### Cell Viability

Wildtype and transfected Panc-1 cells were plated to 24 well plates (n=5) at 50,000 cells per well and placed in incubator for 24 hours. The cells were then treated with either Gemcitabine (Selleckchem, Cat. S1714) or DMSO (Thermo Fisher, Cat. D12345). Cells treated with Gemcitabine were treated at one of the following concentrations: 0 μM, 2.5 μM, 5 μM, 25 μM while cells treated with DMSO were treated at: 0%, 0.025%, 0.05%, or 0.25%. Cells remained in Gemcitabine or DMSO for 72 hours before being evaluated using CellTiter-Glo 2.0 Cell Viability Assay (Promega, Cat. G9241). A 1:1 cell suspension was loaded into a white 96 well plate in triplicate, the plate was then covered with foil and placed on an orbital shaker for 2 minutes, the plate was then left to sit at room temperature for 10 minutes before luminescence was measured using Infinite 2000 PRO microplate reader (Tecan, MAnnedorf, Switzerland).

### Western Blot

Panc-1 cell pellets were collected using a standard RIPA lysis buffer containing protease inhibitor cocktail. Total protein concentration of collected cells was then determined using BCA Protein Assay kit (Pierce, Rockford, IL, USA). The samples were boiled at 95° C. for 10 minutes and then underwent SDS-PAGE on a 10% Mini-PROTEAN TGX Stain-free Protein Gel (BioRad, Cat. 4568033) for 90 min at 100 V. The gel was then transferred to a nitrocellulose membrane using Trans-Blot Turbo Transfer Systems (BioRad, Hercules, CA, USA) with Trans-Blot Turbo RTA Mini 0.2 μm Nitrocellulose Transfer Kit (BioRad Cat. 1704158), mixed molecular weight program, 10 minutes. The blot was placed in 5% milk and blocked at room temperature on shaker for 2 hours. Incubation in primary NRF2 antibody was done overnight at 4° C. on shaker (1:1,000, Abcam ab62352). Membrane was then incubated at room temperature on shaker for 1 hour in NRF2 secondary (1:10,000, Abcam ab205718). The SuperSignal West Dura Extended Duration ECL (Pierce) kit was used on the LI-COR Odyssey FC to visualize bands. Membrane was then incubated overnight at 4° C. on shaker in Gapdh primary antibody (1:10,000, Cell signaling tech cat. 97166), membrane was then incubated at room temperature on shaker for 1 hour in Gapdh secondary (1:1000, thermo fisher Cat. P131430) and SuperSignal West Dura Extended Duration ECL (Pierce) kit was again used on the LI-COR Odyssey FC to visualize bands.

### Results

To develop CRISPR-directed gene editing as an augmentative therapy for solid tumors, we built upon and extended earlier research in which we targeted NRF2 [6, 9, 18]. NRF2 is a master regulator transcription factor which controls the expression of approximately 200 downstream genes in various pathways linked to oncogenesis. In many tumors, the level or activity of NRF2 is elevated in response to outside stress which, of course, includes chemotherapy, radiation therapy, targeted therapy, and immunotherapy [19,20]. Subsequently, pathways that interfere with drug action, often reducing penetration at the cell membrane or influencing intake mechanisms, are engaged. For reference, FIG. 15A illustrates the cellular pathways and response elements that are regulated, at least in part, by the activation of NRF2. Each downstream activity can play a pivotal role in the initiation, maturation, or prolongation of oncogenic phenotypes. Here, we purposely focus on the disrupting downstream regulation of drug resistance enabled by enhanced NRF2 activity. FIG. 15B provides details of NRF2 activity at the molecular level under normal growth conditions, under stressful conditions, that include chemotherapy, and under conditions where gene editing has disrupted NRF2 function.

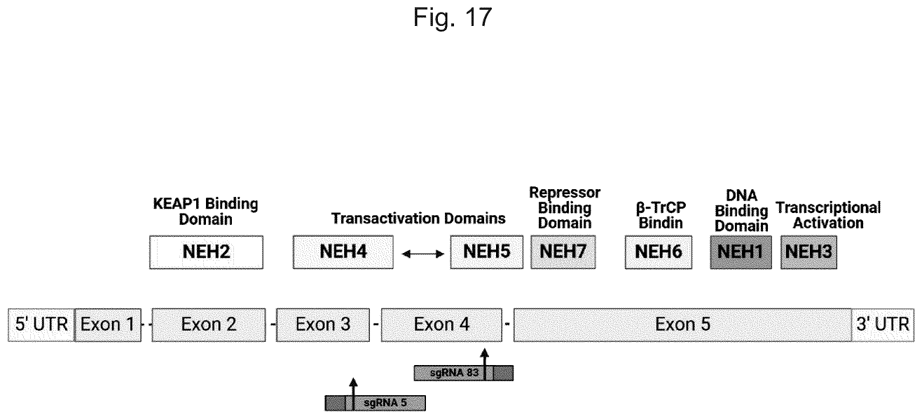

FIG. 17 displays the genetic structure of the NRF2 gene which is divided into five exons cobbled together tightly with each functional protein domain highlighted above its respective exon. For example, exon 3 and exon 4 encode important transactivation domains that are central to the transcriptional activity of this master regulator. We decided to use CRISPR-directed gene editing to disrupt NRF2 within each of these exons precisely because these protein domains play a central role in regulating transcriptional activation of oncogenic pathways. Two sgRNAs were designed to target two sites, one in exon 3 (sg5) and one in exon 4 (sg83); these guide RNAs in their respective target sites are illustrated in FIG. 17. Of particular interest is the sg83 site in exon 4. This site appears to be genetically stable through initiation and prolongation of oncogenesis in many solid tumors. In other words, the DNA target sequence at this site does not exhibit the mutagenic phenotype often found in many tumors which can increase the number of mutations.

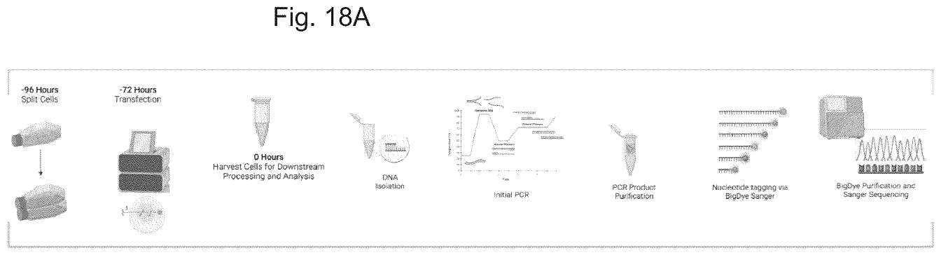

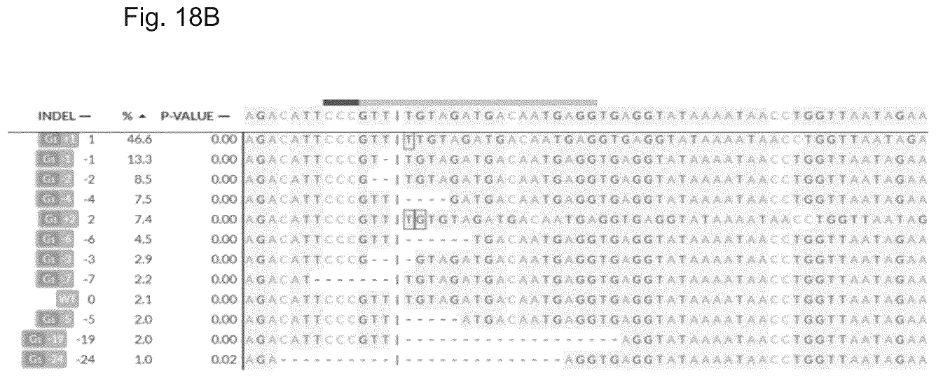

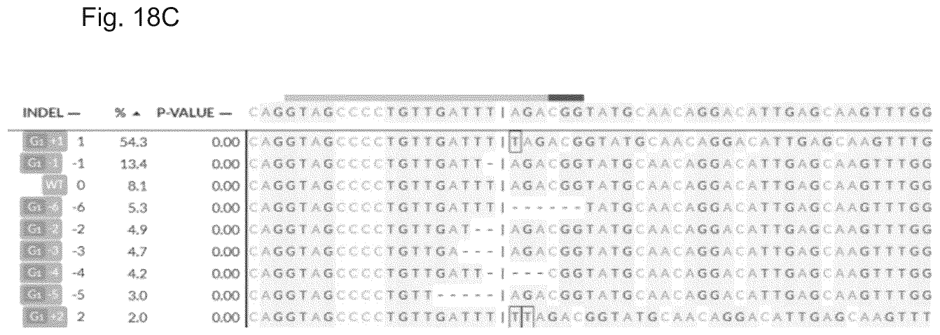

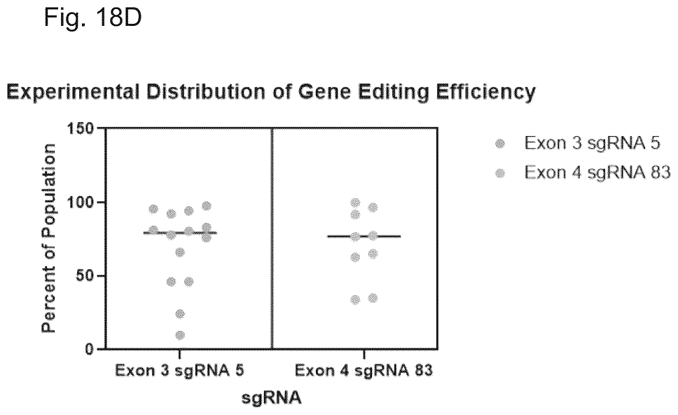

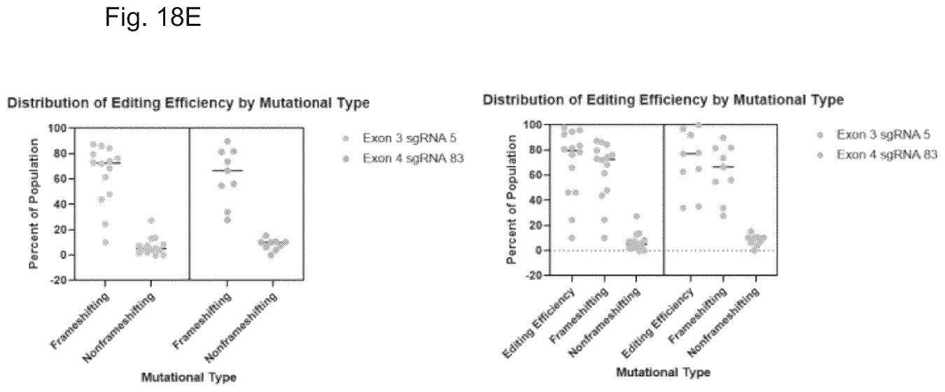

In the first series of experiments, we wanted to evaluate the gene editing capacity directed by sg5 and sg83 respectively. Guide RNAs were introduced into Panc-1 cells as a Ribonucleoprotein Complex (RNP) through suspension in 100 μl of the Lonza via nucleofection [8]. Seventy-two hours afterward, the transfected cells were harvested and processed for Sanger DNA sequencing. The efficacy of gene editing is often taken as a milestone activity, predictive of the efficiency of phenotypic outcome. But, in our experiments, we not only examine basal gene editing efficacy, meaning what percent of the gene is disrupted, but also evaluate the spectrum of insertions and deletions caused by each sgRNA. For example, we know that frameshift mutations (+1. +2, −1, −2 etc.) are more apt to result in significant disruption protein function. FIG. 18A displays the workflow and timeframe of the first level of analyses for CRISPR directed gene editing of NRF2 in Panc1 cells. FIG. 18B presents the indel profile resulting from the action of sg5 while FIG. 18C presents the indel profile generated by sg83. SgRNA complexes are active in disrupting the targeted sites within the gene and importantly, both profiles reveal that a significant level of frameshift mutations have in fact been created, an indicator of impactful functional knockout. While the profiles are similar, it is not surprising that they differ in composition and frequency in types of frameshift and non-frameshift mutations. This diversity of genetic outcomes as a function of CRISPR directed gene editing has been widely reported [10, 22, 23, 29-31].

We took a closer look at the distribution of editing efficacy by repeating this experiment at least nine times; the population of edited cells from each individual experiment is displayed in FIG. 18D. Repetitions of gene editing experiments are crucial since the genetic repair efficiency within each population can vary. Both CRISPR complexes were found to consistently generate high levels of edited cells (greater than 50%) within the population, aligning nicely with earlier data [6, 9, 10, 18, 27]. Once again, we were pleased to see that most of the edited cells contain frameshift mutations (FIG. 18E). Results support the notion that the target sites we have chosen, when genetically disrupted the CRISPR, produce genetic alterations that could have a significant impact on the functional activity of NRF2.

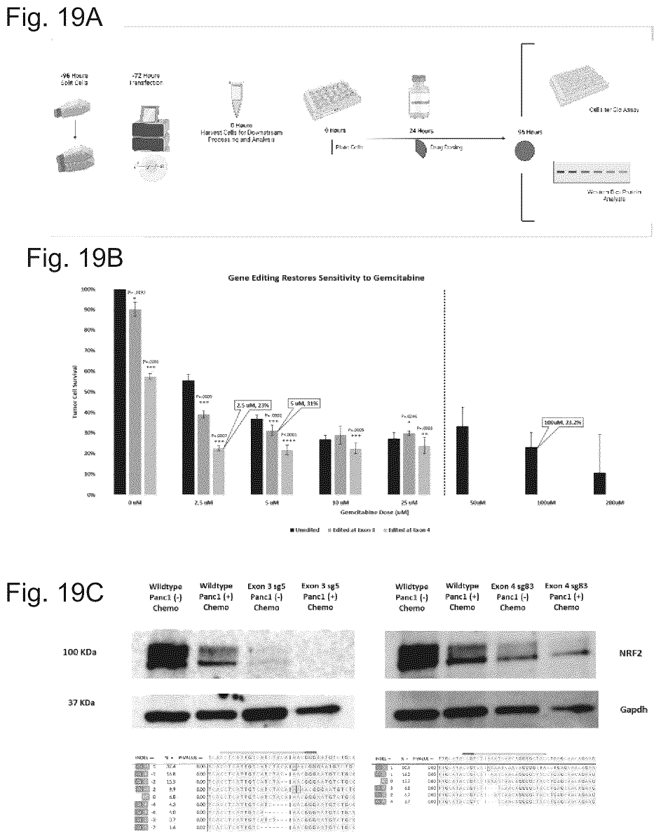

As discussed above, genotype does not always predict phenotype or more importantly, functional outcome, as it relates to cancer therapy. Since our long-term objective is to develop an effective genetic treatment that would augment standard care, we decided to examine the possibility that we could restore sensitivity to gemcitabine by genetic editing of NRF2 in gemcitabine resistant Panc1 cells. We are using gemcitabine as a selection system to determine if gene editing at some level, can help restore tumor cell killing. We established the kill curve of Panc1 unedited cells by gemcitabine under our standard reaction conditions to serve as a baseline. Sg5 which targets a site within exon 3 and sg83 which targets a site within exon 4 were used to disable NRF2 under the same reaction conditions as described above (FIG. 19A). Again, after 72 hours, each cell population was exposed to gemcitabine for an additional 72 hours and thereafter cell viability of the treated and untreated cells was measured using the Cell Titer 2.0 viability assay (Promega, Madison, WI, USA). FIG. 19B displays the results. The black bars represent the standard killing curve of Panc1 cells to gemcitabine. The blue bars indicate the cell viability levels in cells that had been treated with sg5 prior to addition of the chemotherapeutic agent while the green bars indicate the viability levels of a population of cells that had been treated with sg83. Gemcitabine induces cell killing in the absence of gene editing in a traditional downward slope [3, 11, 12], but the degree and magnitude of cell killing is accentuated in cell populations bearing edited cells. Interestingly, targeting exon 4 with sg83 generates approximately 40% cell killing in the absence of gemcitabine alone but, significant enhancement of cell killing is observed by subsequent treatment of low doses of gemcitabine. The right panel of FIG. 19B presents an average of all experimental data with naïve cells treated with various levels of gemcitabine. To achieve 75% killing, the concentration of drug must approach 100 μM. In contrast, 75% killing is achieved when an edited cell population is treated with between 2.5 μM and 5 μM of gemcitabine. These results are shown in the left panel of FIG. 19B. Experimental samples from FIGS. 18D and 18E were subsequently exposed to chemotherapy and the dose curve response of 9 experiments is displayed. These results represent an early indication that genetic disablement of the NRF2 gene enhances the drug sensitivity of Panc1 cells to the killing action of gemcitabine.

To further evaluate the phenotypic outcome of gene editing within panc1 cells targeting exon 3 and exon 4, the RNA levels of downstream genes regulated by NRF2 were examined. NRF2 acts as a transcription factor for glutamate-cysteine ligase catalytic subunit (GCLC) [44], a gene implicated in poor prognosis due to its regulation of cancer cell proliferation and chemotherapy resistance through the ferroptosis pathway [45-48]. GCLC levels are elevated in chemotherapy resistance pancreatic cancer cell lines, however, when GCLC is knocked down, chemosensitivity is restored [45-48].

Protein expression levels were used to confirm sufficient knockdown of NRF2 in edited populations. Western blots comparing unedited populations to populations of edited panc-1 cells targeted with sgRNA5 and sgRNA83 respectively are presented in FIG. 19C. Several samples of cells were also cultured in media dosed with 2.5 μM of Gemcitabine to induce the NRF2 stress response; editing efficiency is displayed below the respective western blot. Both sets of edited populations reveal significant knockdown of protein when compared to the unedited populations, with GAPDH serving as a loading control.

### Example 15. Sg83 LNP 5 In Vivo

### Animal Experiments

All experiments with mice conformed to Animal Welfare guidelines and were performed in accordance with protocols approved by University of Delaware's Institutional Animal Care and Use Committee. H1703 or A549 cell implanted tumors were generated in 6-8-week-old athymic nude female mice (Charles River, Wilmington MA) by subcutaneous implantation of 5×106 cells in BD matrigel (volume ratio 1:1). Tumor growth was measured and estimated using a caliper and calculated as volume (mm3)=(length [mm]×(width [mm])2×0.5. When tumors reached ˜80-150 mm3, mice were separated to experimental groups. Intratumoral injections consisted of 10 μg Cas9 LNP in a total volume of 25 μL purchased from Precision Nanosystems, Vancouver CA). Synthetic single gRNA (sg83—SEQ ID NO:24) was ordered from Synthego (Menlo Park, California, USA). CleanCap® Cas9 mRNA (N1-Methyl Pseudouridine modified) was ordered from TriLink Biotechnologies (San Diego CA). Mice were humanely euthanized, and tumors were excised, homogenized and genomic DNA was isolated for downstream analysis.

### Next Generation Sequencing

Genomic DNA was extracted using Qiagen DNeasy Blood and Tissue kit (Qiagen, Hilden Germany) following the manufacturer's protocol. Genomic DNA was amplified using Phusion Flash High Fidelity PCR Master Mix with HF Buffer (Thermo Fisher, Waltham MA). Parameters were optimized for an amplicon size of 402 bp using primers FWD 5′-GTAGTGGTGCCTTAGAGCTTACTCATCC-3′ (SEQ ID NO:91) and REV 5′-CTAGCATGGGCAGTACTCATGACTAAG-3′ (SEQ ID NO:92) (integrated DNA Technologies, Coralville, Iowa). NGS Amplicon PCR was done with primers modified to include Illumina's overhang adapter sequences. Indexing PCR was done using KAPA HiFi HotStart ReadyMix PCR Kit (Roche Diagnostics, Florham Park NJ), cleaned up using AMPure XP beads (Beckman Coulter, Brea CA) and sequenced using a MiSeq System (Illumina, San Diego CA) with readouts visualized using CRISPResso2.

TABLE 3
A549 cell-based xenograft

| | | | | | | | | | | | Editing |
|---|---|---|---|---|---|---|---|---|---|---|---|
| Sample | Injection | | | | | | | | | Total | Minus Avg |
| Information | Info | WT | +1 | C > T | −1 | −6 | −4 | −2 | Other | Editing | Control |
| 1149 | LNP5-Ex4- | 95.98 | 0.76 | 0.21 | | | | | 3.05 | 4.02 | 1.96 |
| | sg83- 10 μg | | | | | | | | | | |
| 1154 | LNP5-Ex4- | 97.15 | 0.21 | | | | | | 2.64 | 2.85 | 0.79 |
| | sg83- 10 μg | | | | | | | | | | |
| 1137 | LNP5-Ex4- | 94.41 | 1.17 | 0.24 | 0.42 | | | | 3.76 | 5.59 | 3.53 |
| | sg83- 10 μg | | | | | | | | | | |
| 1142 | LNP5-Ex4- | 97.07 | 0.28 | | | | | | 2.65 | 2.93 | 0.87 |
| | sg83- 10 μg | | | | | | | | | | |
| 1144 | LNP5-Ex4- | 95.42 | 0.82 | 0.28 | 0.23 | | | | 3.25 | 4.58 | 2.52 |
| | sg83- 10 μg | | | | | | | | | | |
| 1147 | LNP5-Ex4- | 83.65 | 4.59 | | 1.39 | 0.74 | 0.58 | 0.49 | 8.56 | 16.35 | 14.29 |
| | sg83- 10 μg | | | | | | | | | | |
| 1148 | LNP5-Ex4- | 97.51 | | 0.23 | | | | | 2.26 | 2.49 | 0.43 |
| | sg83- 10 μg | | | | | | | | | | |
| | Average | 94.46 | 1.31 | 0.24 | 0.68 | 0.74 | 0.58 | 0.49 | 1.51 | 5.54 | 3.49 |

TABLE 4
A549 cell-based xenograft

| | | | | | | | | | | | Editing |
|---|---|---|---|---|---|---|---|---|---|---|---|
| Sample | Injection | | | | | | | | | Total | Minus Avg |
| Information | Info | WT | +1 | C > T | −1 | −6 | −4 | −2 | Other | Editing | Control |
| 1153 | LNP5-Ex4- | 96.91 | 0.37 | 0.23 | | | | | 2.49 | 3.09 | 1.03 |
| | sg83- 10 μg | | | | | | | | | | |
| 1155 | LNP5-Ex4- | 92.48 | 1.79 | | 0.57 | 0.29 | 0.26 | 0.21 | 4.4 | 7.52 | 5.46 |
| | sg83- 10 μg | | | | | | | | | | |
| 1151 | LNP5-Ex4- | 95.31 | 0.98 | | 0.32 | | | | 3.39 | 4.69 | 2.63 |
| | sg83- 10 μg | | | | | | | | | | |
| 1138 | LNP5-Ex4- | 96.65 | 0.42 | 0.24 | | | | | 2.69 | 3.35 | 1.29 |
| | sg83- 10 μg | | | | | | | | | | |
| 1152 | LNP5-Ex4- | 91.5 | 2.14 | 0.24 | 0.62 | | | 0.41 | 5.09 | 8.5 | 6.44 |
| | sg83- 10 μg | | | | | | | | | | |
| 1156 | LNP5-Ex4- | 97.99 | | 0.21 | | | | | 1.8 | 2.01 | −0.05 |
| | sg83- 10 μg | | | | | | | | | | |
| 1158 | LNP5-Ex4- | 80.8 | 5.1 | | 2.31 | 0.89 | 0.81 | 0.8 | 9.29 | 19.2 | 17.14 |
| | sg83- 10 μg | | | | | | | | | | |
| | Average | 93.09 | 1.80 | 0.23 | 0.96 | 0.59 | 0.54 | 0.47 | 2.33 | 6.91 | 4.85 |

TABLE 5
A549 cell-based xenograft

| | | | | | | | | | | | Editing |
|---|---|---|---|---|---|---|---|---|---|---|---|
| Sample | Injection | | | | | | | | | Total | Minus Avg |
| Information | Info | WT | +1 | C > T | −1 | −6 | −4 | −2 | Other | Editing | Control |

| | | | | | |
|---|---|---|---|---|---|
| 1275 | LNP5-Ex4- | 97.5 | 2.5 | 2.5 | −0.36 |
| | sg83- 10 | | | | |
| | μg; 2x Inj | | | | |
| 1276 | LNP5-Ex4- | 97.38 | 2.62 | 2.62 | −0.24 |
| | sg83- 10 | | | | |
| | μg; 2x Inj | | | | |
| 1319 | LNP5-Ex4- | 96.25 | 3.75 | 3.75 | 0.89 |
| | sg83-10 | | | | |
| | μg; 2x Inj | | | | |
| 1321 | LNP5-Ex4- | 54.87 | 45.13 | 45.13 | 42.27 |
| | sg83- 10 | | | | |
| | μg; 2x Inj | | | | |
| 1322 | LNP5-Ex4- | 95.14 | 4.86 | 4.86 | 2.00 |
| | sg83-10 | | | | |
| | μg; 2x Inj | | | | |
| | Average | 88.23 | | 11.17 | 9.71 |

Tables 3-5 show three separate experimental groups and the respective editing profile and overall editing outcomes as analyzed by CRISPResso. From left to right, the wildtype or unedited (WT) percentage is displayed showing anywhere from 54-98% for each respective sample, followed by a breakdown of the individual insertion or deletions (indels) present noted as +1, C>T, −1, −6, −4, −2 or other. The last two columns present total editing efficiency and total editing efficiency normalized to a control sample. Overall, the editing efficiency in vivo after intratumoral injection of a CRISPR/Cas9 LNP targeting exon 4 of NRF2 shows anywhere from 2.5-45% editing.

TABLE 6
H1703 44-25 cell-based xenograft

| | | | | | | | | | | | Editing |
|---|---|---|---|---|---|---|---|---|---|---|---|
| Sample | Injection | | | | | | | | | Total | Minus Avg |
| Information | Info | WT | +1 | C > T | −1 | −6 | −4 | −2 | Other | Editing | Control |

| | | | | | |
|---|---|---|---|---|---|
| 1159 | LNP5-Ex4- | 96.98 | 3.02 | 3.02 | −0.09 |
| | sg83 10 μg | | | | |
| 1162 | LNP5-Ex4- | 92.59 | 7.41 | 7.41 | 4.30 |
| | sg83 10 μg | | | | |
| 1163 | LNP5-Ex4- | 63.68 | 36.32 | 36.32 | 33.21 |
| | sg83 10 μg | | | | |
| 1167 | LNP5-Ex4- | 86.94 | 13.06 | 13.06 | 9.95 |
| | sg83 10 μg | | | | |
| 1168 | LNP5-Ex4- | 96.75 | 3.25 | 3.25 | 0.14 |
| | sg83 10 μg | | | | |
| 1234 | LNP5-Ex4- | 66.19 | 33.81 | 33.81 | 30.70 |
| | sg83 10 μg | | | | |
| 1242 | LNP5-Ex4- | 86.27 | 13.73 | 13.73 | 10.62 |
| | sg83 10 μg | | | | |
| 1243 | LNP5-Ex4- | 97.54 | 2.46 | 2.46 | −0.65 |
| | sg83 10 μg | | | | |
| 1246 | LNP5-Ex4- | 93.61 | 6.39 | 6.39 | 3.28 |
| | sg83 10 μg | | | | |
| | Average | 86.73 | 13.27 | 13.27 | 11.21 |

Table 6 shows the respective editing profile and overall editing outcomes as analyzed by CRISPResso. From left to right, the wildtype or unedited (WT) percentage is displayed showing anywhere from 54-98% for each respective sample, followed by a breakdown of the individual insertion or deletions (indels) present noted as +1, C>T, −1, −6, −4, −2 or other. The last two columns present total editing efficiency and total editing efficiency normalized to a control sample. Overall, the editing efficiency in vivo after intratumoral injection of a CRISPR/Cas9 LNP targeting exon 4 of NRF2 shows anywhere from 2.46-36.32% editing.

### Example 16. Efficacy of Targeting Across Exon 4

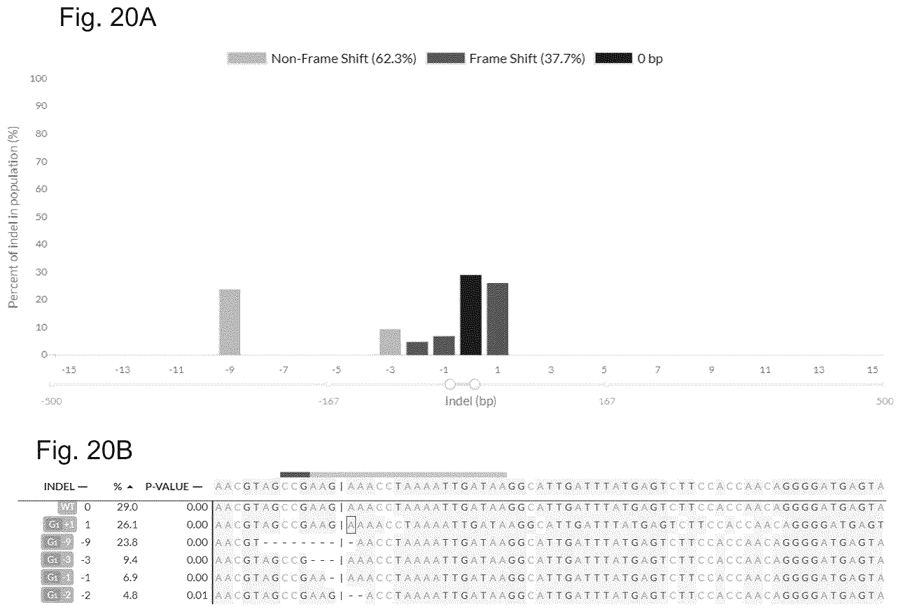

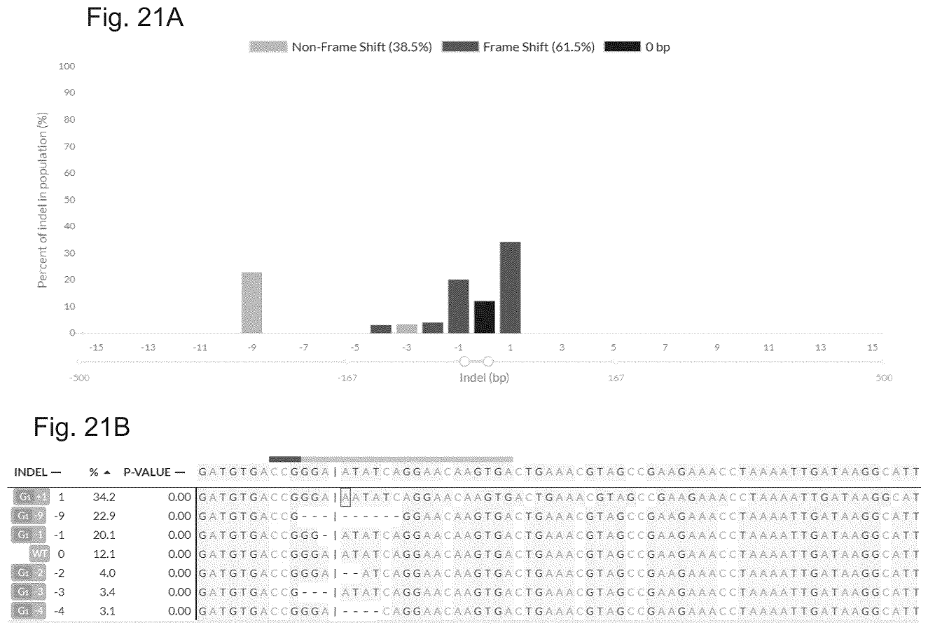

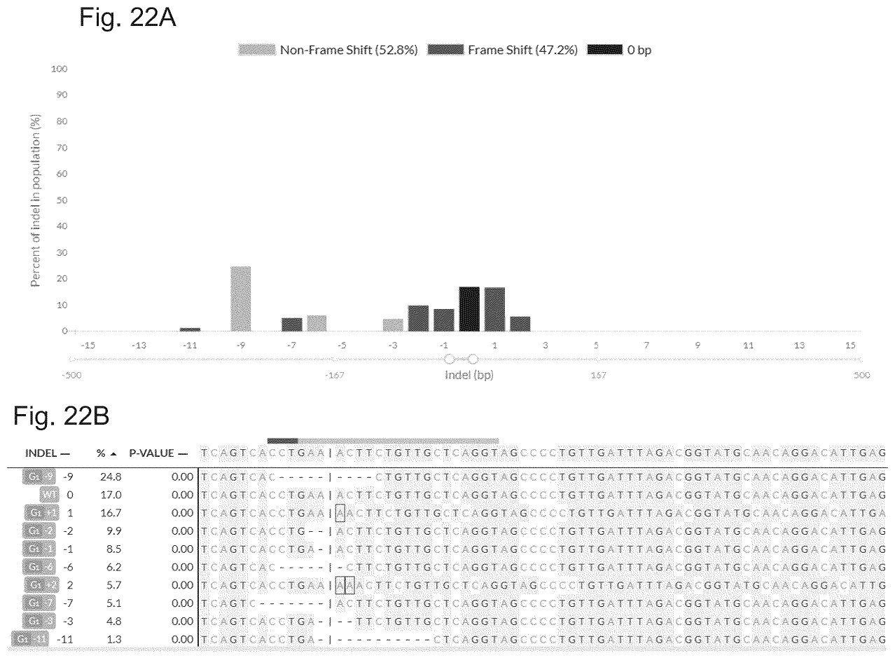

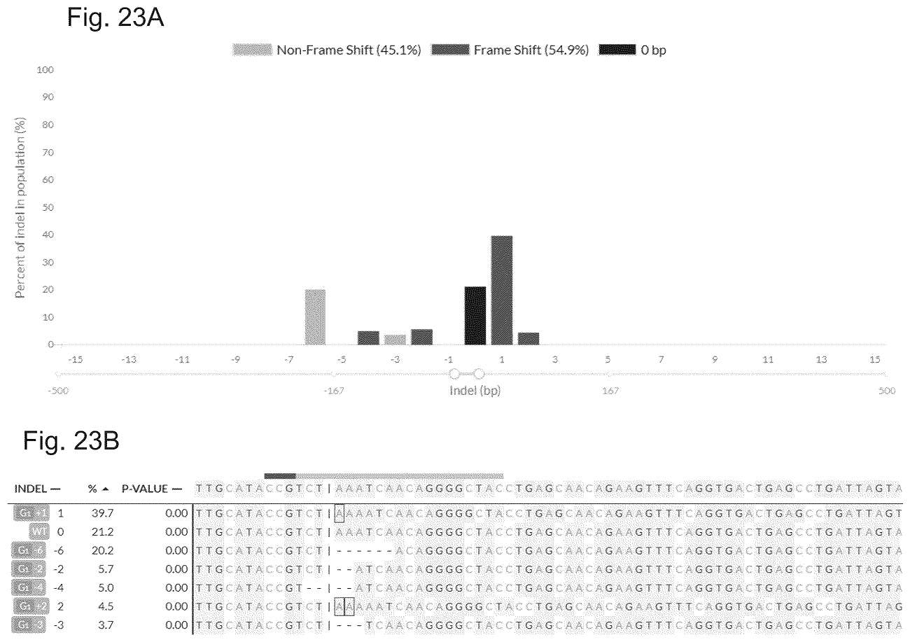

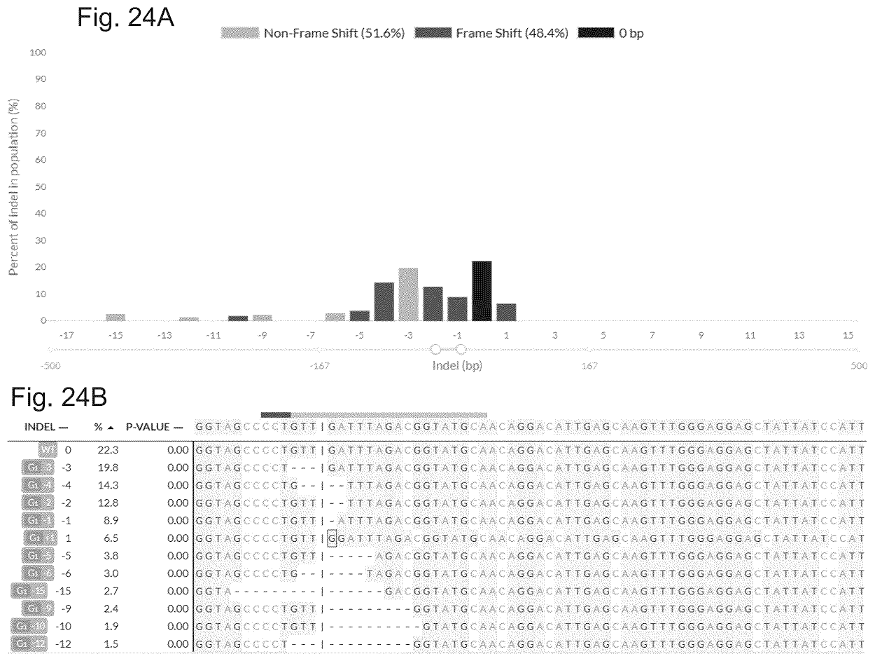

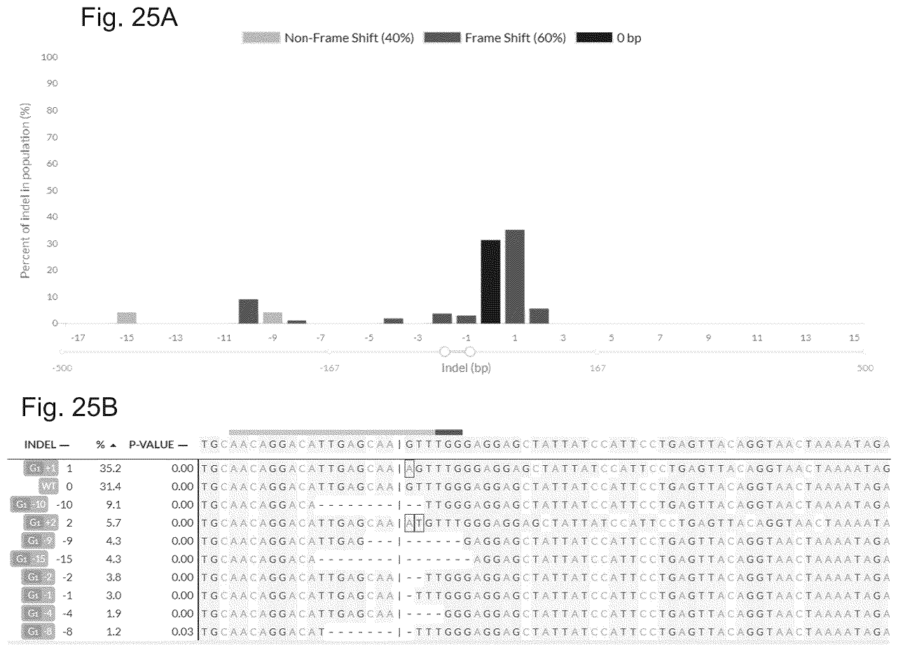

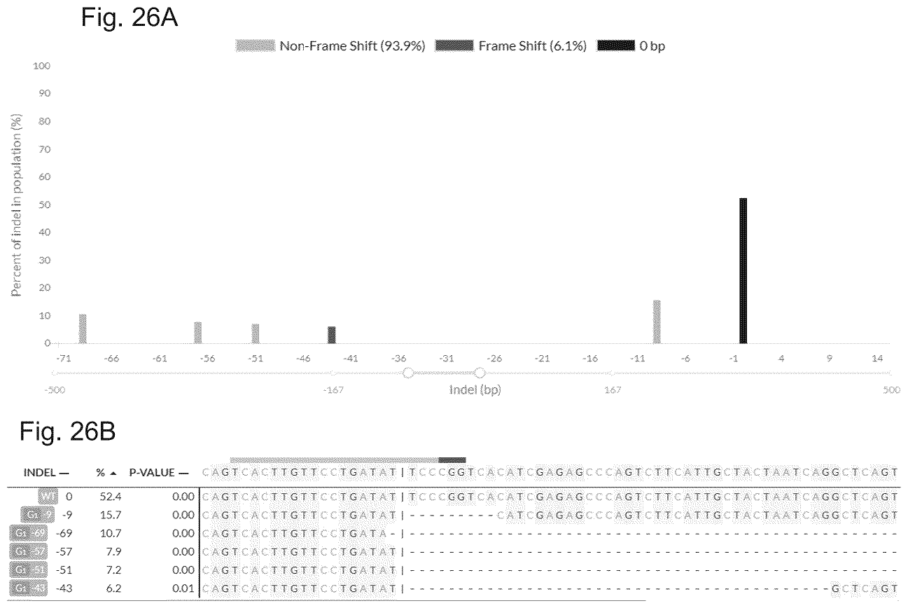

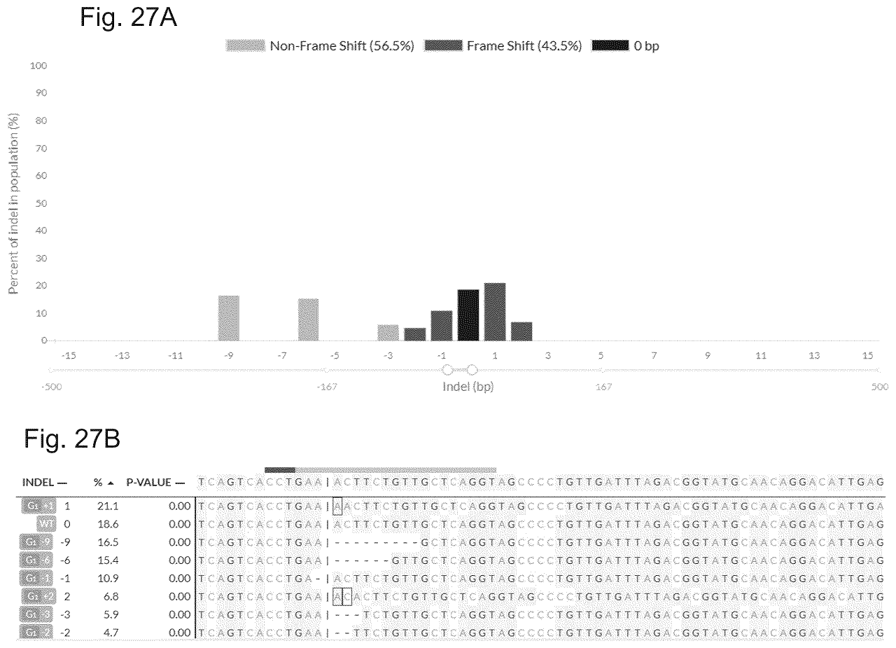

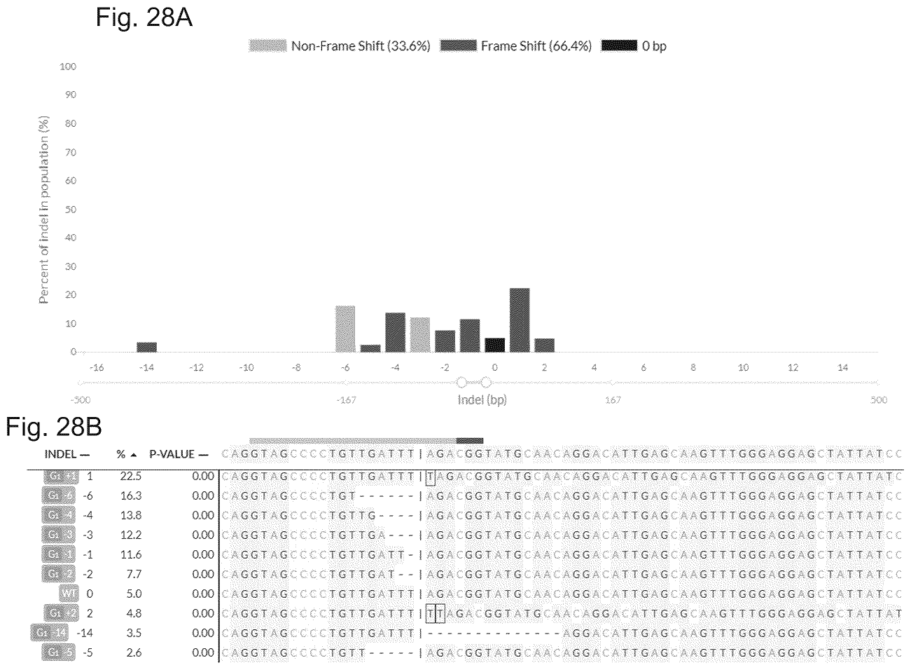

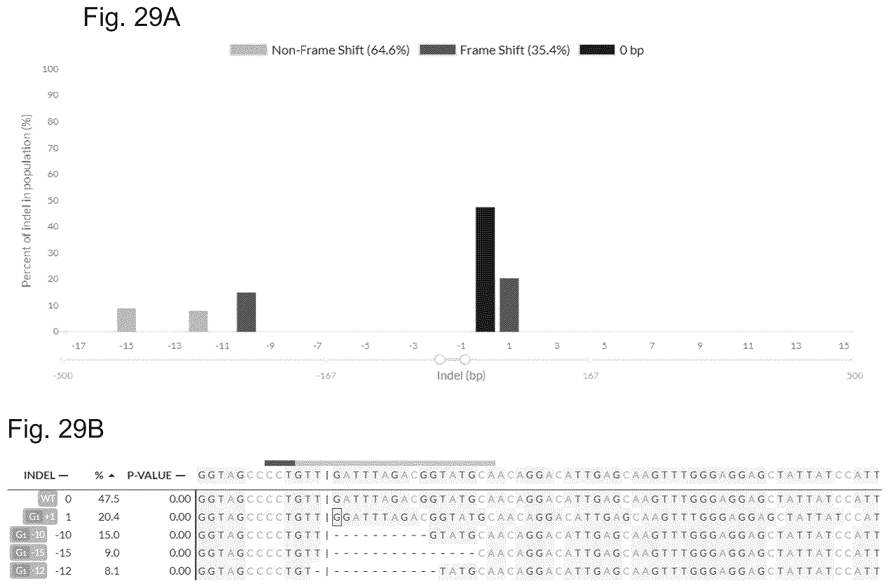

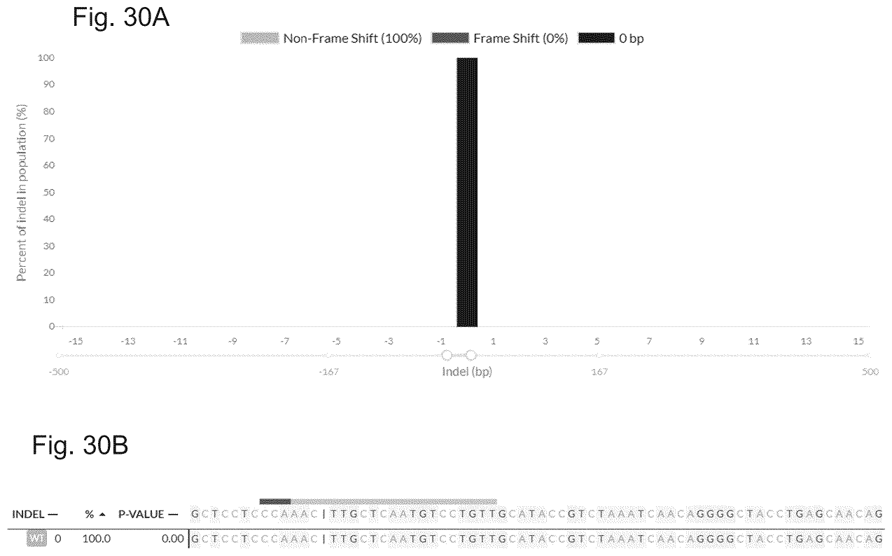

To test efficacy of targeting across exon 4 of NRF2, multiple gRNAs were designed spanning the entirety of exon 4. Briefly, A549 or H1703 cells were cultured and plated 24 hours before transfection with each respective gRNA and Cas9 mRNA delivered using lipofection-based MessengerMax. Cells were collected and genomic DNA was isolated. Exon 4 of NRF2 was PCR amplified and Sanger sequenced. Sanger sequencing from each sample was assessed for indel efficiency at the CRISPR target site, using DECODR software to deconvolute sequence chromatograms. FIGS. 20-25 shows the DECODR analysis output of an individual experiment after testing each respective gRNA in A549 cells. FIGS. 26-30 show the DECODR analysis output of an individual experiment after testing each respective gRNA in H1703 cells. Overall editing efficiency ranged from 52.5-95% within exon 4 of NRF2.

### REFERENCES

1. Vomund, S., Schäfer, A., Parnham, M. J., Brüne, B. & Von Knethen, A. Nrf2, the Master Regulator of Anti-Oxidative Responses. International Journal of Molecular Sciences 2017, Vol. 18, Page 2772 18, 2772 (2017).
2. Baird, L. & Dinkova-Kostova, A. T. The cytoprotective role of the Keap1-Nrf2 pathway. Arch Toxicol 85, 241-272 (2011).
3. Uruno, A. & Motohashi, H. The Keap1-Nrf2 system as an in vivo sensor for electrophiles. Nitric Oxide 25, 153-160 (2011).
4. Härkönen, J. et al. A pan-cancer analysis shows immunoevasive characteristics in NRF2 hyperactive squamous malignancies. Redox Biol 61, 102644 (2023).
5. Zimta, A. A. et al. The role of Nrf2 activity in cancer development and progression. Cancers vol. 11 Preprint at https://doi.org/10.3390/cancers11111755 (2019).
6. Denicola, G. M. et al. Oncogene-induced Nrf2 transcription promotes ROS detoxification and tumorigenesis. Nature 475, 106 (2011).
7. Satoh, H., Moriguchi, T., Takai, J., Ebina, M. & Yamamoto, M. Nrf2 prevents initiation but accelerates progression through the kras signaling pathway during lung carcinogenesis. Cancer Res 73, 4158-4168 (2013).
8. Denicola, G. M. et al. Oncogene-induced Nrf2 transcription promotes ROS detoxification and tumorigenesis. Nature 2011 475:7354 475, 106-109 (2011).
9. Xia, D. et al. Nrf2 promotes esophageal squamous cell carcinoma (ESCC) resistance to radiotherapy through the CaMKIIα-Associated activation of autophagy. Cell Biosci 10, 1112 (2020).
10. Sánchez-Ortega, M., Carrera, A. C. & Garrido, A. Role of NRF2 in Lung Cancer. Cells 10, 1879 (2021).
11. Zhao, J. et al. Nrf2 Mediates Metabolic Reprogramming in Non-Small Cell Lung Cancer. Front Oncol 10, (2020).
12. Pölönen, P. et al. Nrf2 and SQSTM1/p62 jointly contribute to mesenchymal transition and invasion in glioblastoma. Oncogene 2019 38:50 38, 7473-7490 (2019).
13. Awuah, W. A. et al. Exploring the role of Nrf2 signaling in glioblastoma multiforme. Discover Oncology 13, 1-12 (2022).
14. Godoy, P. R. D. V., Pour Khavari, A., Rizzo, M., Sakamoto-Hojo, E. T. & Haghdoost, S. Targeting NRF2, Regulator of Antioxidant System, to Sensitize Glioblastoma Neurosphere Cells to Radiation-Induced Oxidative Stress. Oxid Med Cell Longev 2020, (2020).
15. Cykowiak, M. & Krajka-Kuźiak, V. Role of Nrf2 in Pancreatic Cancer. Antioxidants 11, (2022).
16. Chio, I. I. C. et al. NRF2 Promotes Tumor Maintenance by Modulating mRNA Translation in Pancreatic Cancer. Cell 166, 963-976 (2016).
17. Matsumoto, R. et al. Nrf2 Depletion Sensitizes Pancreatic Cancer Cells to Gemcitabine via Aldehyde Dehydrogenase 3a1 Repression. Journal of Pharmacology and Experimental Therapeutics 379, 33-40 (2021).
18. Head and Neck Cancer Statistics. Cancer. Net https://www.cancer.net/cancer-types/head-and-neck-cancer/statistics.
19. Sung, H. et al. Global Cancer Statistics 2020: GLOBOCAN Estimates of Incidence and Mortality Worldwide for 36 Cancers in 185 Countries. CA Cancer J Clin 71, 209-249 (2021).
20. Gormley, M., Creaney, G., Schache, A., Ingarfield, K. & Conway, D. I. Reviewing the epidemiology of head and neck cancer: definitions, trends and risk factors. Br Dent J 233, 780-786 (2022).
21. Johnson, D. E. et al. Head and neck squamous cell carcinoma. Nature Reviews Disease Primers vol. 6 Preprint at https://doi.org/10.1038/s41572-020-00224-3 (2020).
22. Picon, H. & Guddati, A. K. Mechanisms of resistance in head and neck cancer. Am J Cancer Res 10, 2742-2751 (2020).
23. Min, H.-Y. & Lee, H.-Y. Mechanisms of resistance to chemotherapy in non-small cell lung cancer. Arch Pharm Res 44, 146-164 (2021).
24. Lagergren, J., Smyth, E., Cunningham, D. & Lagergren, P. Oesophageal cancer. Lancet 390, 2383-2396 (2017).
25. Kamangar, F. et al. The global, regional, and national burden of oesophageal cancer and its attributable risk factors in 195 countries and territories, 1990-2017: a systematic analysis for the Global Burden of Disease Study 2017. Lancet Gastroenterol Hepatol 5, 582-597 (2020).
26. Ostrom, Q. T. et al. CBTRUS Statistical Report: Primary Brain and Other Central Nervous System Tumors Diagnosed in the United States in 2012-2016. Neuro Oncol 21, V1-V100 (2019).
27. Zhang, H. et al. Glioblastoma Treatment Modalities besides Surgery. J Cancer 10, 4793 (2019).
28. Singh A, Misra V, Thimmulappa R K, Lee H, Ames S, Hoque M O, et al. Dysfunctional KEAP1-NRF2 interaction in non-small-cell lung cancer. Meyerson M, editor. PLoS Med. 2006; 3:1865-76.
29. Solis L M, Behrens C, Dong W, Suraokar M, Ozburn N C, Moran C A, et al. Nrf2 and Keap1 abnormalities in non-small cell lung carcinoma and association with clinicopathologic features. Clinical Cancer Research. 2010; 16:3743-53.
30. Yang H, Wang W, Zhang Y, Zhao J, Lin E, Gao J, et al. The Role of NF-E2-Related Factor 2 in Predicting Chemoresistance and Prognosis in Advanced Non-Small-Cell Lung Cancer. Clin Lung Cancer. Elsevier; 2011; 12:166-71.
31. Bialk P, Wang Y, Banas K, Kmiec E B. Functional Gene Knockout of NRF2 Increases Chemosensitivity of Human Lung Cancer A549 Cells in Vitro and in a Xenograft Mouse Model. Mol Ther Oncolytics. 2018; 11:75-89.
32. Rivera-Torres N, Bialk P, Kmiec E B. CRISPR-Directed Gene Editing as a Method to Reduce Chemoresistance in Lung Cancer Cells. Methods in Molecular Biology. Humana Press Inc.; 2023; 2660:263-71.
33. Banas K, Modarai S, Rivera-Torres N, Yoo B C, Bialk P A, Barrett C, et al. Exon skipping induced by CRISPR-directed gene editing regulates the response to chemotherapy in non-small cell lung carcinoma cells. Gene Ther; 2022; 29:357-67.
34. Kerins M J, Ooi A. A catalogue of somatic NRF2 gain-of-function mutations in cancer. Sci Rep. Nature Publishing Group; 2018; 8.
35. Banas K, Rivera-Torres N, Bialk P, Yoo B C, Kmiec E B, Kmiec E B. Kinetics of nuclear uptake and site-specific DNA cleavage during crispr-directed gene editing in solid tumor cells. Molecular Cancer Research. American Association for Cancer Research Inc.; 2020; 18:891-902.
36. Rangan S R S. A new human cell line (FaDu) from a hypopharyngeal carcinoma. Cancer. 1972; 29:117-21.
37. Johnson D E, Burtness B, Leemans C R, Lui V W Y, Bauman J E, Grandis J R. Head and neck squamous cell carcinoma. Nat Rev Dis Primers. Nature Research; 2020.
38. Fuereder T. Essential news of current guidelines: head and neck squamous cell carcinoma. Memo—Magazine of European Medical Oncology. Springer; 2022. page 278-81.
39. Wise-Draper T M, Bahig H, Tonneau M, Karivedu V, Burtness B. Current Therapy for Metastatic Head and Neck Cancer: Evidence, Opportunities, and Challenges. American Society of Clinical Oncology Educational Book. American Society of Clinical Oncology (ASCO); 2022; 527-40.
40. Wang XJXJX-J, Sun Z, Villeneuve NFNF, Zhang S, Zhao F, Li Y, et al. Nrf2 enhances resistance of cancer cells to chemotherapeutic drugs, the dark side of Nrf2. Carcinogenesis. 2008; 29:1235-43.
41. Shimada Y, Imamura M, Wagata T, Yamaguchi N, Tobe T. Characterization of 21 newly established esophageal cancer cell lines. Cancer. 1992; 69:277-84.
42. Shen L-Y, Wang H, Dong B, Yan W-P, Lin Y, Shi Q, et al. Possible prediction of the response of esophageal squamous cell carcinoma to neoadjuvant chemotherapy based on gene expression profiling. 2015; 7.
43. Sansbury B M, Hewes A M, Kmiec E B. Understanding the diversity of genetic outcomes from CRISPR-Cas generated homology-directed repair. Commun Biol. Springer US; 2019; 2:1-10.
44. Bialk P, Sansbury B, Rivera-Torres N, Bloh K, Man D, Kmiec E B. Analyses of point mutation repair and allelic heterogeneity generated by CRISPR/Cas9 and single-stranded DNA oligonucleotides. Sci Rep [Internet]. Nature Publishing Group; 2016; 6:32681.

What is claimed is:

1. A method of reducing NRF2 expression or activity in a cell comprising introducing into the cell (a) one or more DNA sequences encoding a gRNA comprising the sequence set forth in any one of SEQ ID NO: 3-74 and (b) a nucleic acid sequence encoding a CRISPR-associated endonuclease, whereby the gRNA hybridizes to the NRF2 gene and the CRISPR-associated endonuclease cleaves the NRF2 gene, and wherein NRF2 expression or activity is reduced in the cell relative to a cell in which the one or more DNA sequences encoding the gRNA and the nucleic acid sequence encoding the CRISPR-associated endonuclease are not introduced.

2. The method of claim 1, wherein the gRNA comprises a tracrRNA and a crRNA.

3. The method of claim 1, wherein the gRNA is a single gRNA.

4. The method of claim 1, wherein the CRISPR-associated endonuclease is a class 2 CRISPR-associated endonuclease.

5. The method of claim 4, wherein the class 2 CRISPR-associated endonuclease is Cas9 or Cas12a.

6. The method of claim 1, wherein the cell is a cancer cell.

7. The method of claim 6, wherein the cancer cell is selected from the group consisting of a lung cancer cell, a head and neck cancer cell, an esophageal cancer cell, a glioma cell, a pancreatic cancer cell, a cervical cancer cell, a breast cancer cell, a uterine cancer cell, a renal cell cancer cell, a liver cancer cell, a bladder cancer cell, a colorectal cancer cell, and a melanoma cell.

8. The method of claim 6, further comprising the step of introducing into the cancer cell one or more chemotherapeutic agents.

9. The method of claim 8, wherein the one or more chemotherapeutic agents are selected from the group consisting of a topoisomerase II inhibitor, a mitotic inhibitor, an alkylating agent, an antimetabolite, a topoisomerase I inhibitor, a platinum compound/complex, an immunotherapy agent, and a combination thereof.

10. The method of claim 1, wherein the gRNA comprises the sequence set forth in SEQ ID NO:16 or SEQ ID NO:24.

11. A method of reducing NRF2 expression or activity in a cell comprising introducing into the cell (a) a guide RNA (gRNA) comprising the sequence set forth in any one of SEQ ID NO: 3-74, and (b) a CRISPR-associated endonuclease, whereby the one or more gRNAs hybridize to the NRF2 gene and the CRISPR-associated endonuclease cleaves the NRF2 gene, and wherein NRF2 expression or activity is reduced in the cell relative to a cell in which the gRNA and the CRISPR-associated endonuclease are not introduced.

12. The method of claim 11, wherein the gRNA comprises a tracrRNA and a crRNA.

13. The method of claim 11, wherein the gRNA is a single gRNA.

14. The method of claim 11, wherein the CRISPR-associated endonuclease is a class 2 CRISPR-associated endonuclease.

15. The method of claim 14, wherein the class 2 CRISPR-associated endonuclease is Cas9 or Cas12a.

16. The method of claim 11, wherein the cell is a cancer cell.

17. The method of claim 16, wherein the cancer cell is selected from the group consisting of a lung cancer cell, a head and neck cancer cell, an esophageal cancer cell, a glioma cell, a pancreatic cancer cell, a cervical cancer cell, a breast cancer cell, a uterine cancer cell, a renal cell cancer cell, a liver cancer cell, a bladder cancer cell, a colorectal cancer cell, and a melanoma cell.

18. The method of claim 16, further comprising the step of introducing into the cancer cell one or more chemotherapeutic agents.

19. The method of claim 18, wherein the one or more chemotherapeutic agents are selected from the group consisting of a topoisomerase II inhibitor, a mitotic inhibitor, an alkylating agent, an antimetabolite, a topoisomerase I inhibitor, a platinum compound/complex, an immunotherapy agent, and a combination thereof.

20. The method of claim 11, wherein the gRNA comprises the sequence set forth in SEQ ID NO:16 or SEQ ID NO:24.

21. A gRNA comprising the sequence set forth in any one of SEQ ID NO: 3-74.

22. The gRNA of claim 21, wherein the gRNA comprises the sequence set forth in SEQ ID NO:16 or SEQ ID NO:24.

23. A pharmaceutical composition comprising the gRNA of claim 21 and a pharmaceutically acceptable carrier.

24. The pharmaceutical composition of claim 23, further comprising a CRISPR-associated endonuclease.

25. The pharmaceutical composition of claim 23, further comprising one or more chemotherapeutic agents.

26. The pharmaceutical composition of claim 25, wherein the one or more chemotherapeutic agents are selected from the group consisting of a topoisomerase II inhibitor, a mitotic inhibitor, an alkylating agent, an antimetabolite, a topoisomerase I inhibitor, a platinum compound/complex, an immunotherapy agent, and a combination thereof.

27. An RNP complex comprising the gRNA of claim 21 and a CRISPR-associated endonuclease.

28. A pharmaceutical composition comprising the RNP complex of claim 27 and a pharmaceutically acceptable carrier.

29. A DNA sequence encoding the gRNA of claim 21.

30. A vector comprising the DNA sequence of claim 29.
